(19) United States
(12) Patent Application Publication
(10) Pub. No.: US 2007/0003947 A1
(43) Pub. Date: Jan. 4, 2007

(54) Detecting genetic risk for periodontal disease

(76) Inventor: Arthur Anthony DeCarlo, Vestavia Hills, AL (US)

Correspondence Address: Dr. Arthur DeCarlo, 609 Park Lake Circle, Birmingham, AL, 35242, US

(21) Appl. No.: 11/331,572

(22) Filed: Jan. 13, 2006

Related U.S. Application Data
(60) Provisional application No. 60/644,733, filed on Jan. 18, 2005.

(51) Int. Cl. C12Q 1/68; C12P 19/34

(52) U.S. Cl. 435/6; 435/91.2

(57) ABSTRACT

A method and kit for the identification of a user's genotypes associated with increased risk for developing periodontal disease are disclosed. The kit includes non-invasive DNA sample collecting means, and disclosed are means for determining certain genotypes which are then compared to those of controls with known periodontal disease status to determine a user's relative risk for developing periodontal disease.

### CROSS-REFERENCES TO RELATED APPLICATIONS

A claim of domestic priority to U.S. Provisional Application No. 60/644,733 (filed Jan. 18th, 2005) is made. This application was filed on a date sufficient to claim priority to the 60/644,733 application.

### STATEMENT REGARDING FEDERALLY SPONSORED RESEARCH OR DEVELOPMENT

This research was sponsored by the National Institutes of Health, and so the federal government maintains certain rights.

### FIELD OF INVENTION

This invention relates generally to a method of detecting a predisposition for developing chronic periodontal disease. Specifically, the invention relates to a method of assessing a user's relative risk for developing chronic periodontal disease, said method relating to analysis of polymorphisms in genes for plasminogen activators and plasminogen activator inhibitors. More specifically, the invention relates to a method of assessing relative risk for developing chronic periodontal disease, said method relating to analysis of polymorphisms in the gene encoding urokinase (uPA) and by analysis of polymorphisms in the gene encoding plasminogen activator inhibitor-1 (PAI-1). Most specifically, the invention relates to a method of assessing relative risk for developing chronic periodontal disease, said method relating to analysis of gene polymorphisms in the 3′ untranslated regions of the genes encoding urokinase (uPA) and the plasminogen activator inhibitor-1 (PAI-1).

### BACKGROUND OF INVENTION

Background of Periodontal Disease

Periodontitis is defined as the loss of tooth support resulting from a microbial challenge in the region surrounding the teeth called the periodontium. Chronic periodontitis develops in association with an immune response resulting from bacterial colonization of the teeth (Haffajee, Socransky et al. 1988). Periodontitis is characterized by degradation of the ligament that supports the tooth in association with epithelial invasion of the ligament space. Histologically, the periodontal lesion demonstrates an increased density of the microvasculature in the gingival connective tissues subjacent to the dental plaque and junctional epithelium (Egelberg 1966; Zoellner and Hunter 1991; Zoellner and Hunter 1994; Pinchback, Taylor et al. 1996).

The majority of adults worldwide have some degree of periodontitis and up to 10% of the world's population suffers more severely from this chronic disease which can involve acute episodes of abscess formation and loss of teeth (Loe, Anerud et al. 1986). According to the American Academy of Periodontology, more than one in three people over age 30 have periodontitis. By a conservative estimate, 35.7 million adults in the United States have periodontitis. The estimated annual cost of basic periodontal therapy in the USA in 1989 was US$ 6 billion (Oliver, Brown et al. 1989) rising to $14.3 billion in 1999 (NIH data May 2003). A February 2000 article in Dental Economics claims that four out of five adult Americans are afflicted with moderate to severe periodontitis, with approximately 50% of 55-64 years old having evidence of severe disease (data from NIH May 2003).

The microbial challenge associated with the onset and progression of chronic periodontitis (inflammation of the periodontium) is also considered to have systemic sequellae such as the exacerbation of diabetes mellitus and increased severity of cardiovascular disease, among others (Grossi, Skrepcinski et al. 1997; Beck, Offenbacher et al. 1998; Offenbacher, Beck et al. 1998). In the developing world, where dental care of any type is limited, periodontitis represents a much more widespread threat to public health.

Background of the Plasminogen Activator System

Both uPA and tPA are secreted in a single-chain (sc-) inactive form but can be activated through limited extracellular proteolysis by plasmin (Petersen, Lund et al. 1988). Therefore, small amounts of plasmin can initiate a feed-back cascade of sc-PA and further plasminogen activation. uPA is capable of association with the membrane by high-affinity binding to the uPA receptor (uPAR) (CD 87) (Vassalli, Baccino et al. 1985) and, possibly, by low-affinity, high capacity binding (Longstaff, Merton et al. 1999). Binding of uPA is mediated by the NH₂-terminal domain of uPA while proteinase activity is mediated by the CH₂-terminal domain of the uPA molecule (Appella, Robinson et al. 1987). Active and inactive forms of uPA can bind the uPAR and activation of uPA may occur in the bound or unbound uPA (Appella, Robinson et al. 1987; Rolden, Cubellis et al. 1990). uPA remains active while bound to its cell receptor and remains active on the cell surface for several hours (Vassalli, Baccino et al. 1985). It was recently demonstrated that blocking uPA binding to its receptor (uPAR) on endothelial cells prevented microvascular tubule formation which was independent of uPAR activation (Kroon, Koolwijk et al. 1999). It is likely that this association with the cell surface regulates and localizes PA activity as well as MMP activity by localizing MMP activation.

There are three significant inhibitors of the PAs. These are the plasminogen activator inhibitor-1 (PAI-1), PAI-2, and protease nexin-1 inhibitor (Mignatti and Rifkin 1993). PAI-1 is a 45 kDa protein which binds the activated PAs tightly (Kd=10⁻⁷ M). PAI-1 is present in serum and platelets and is associated in some cases with the ECM. PAI-2 is a 47 kDa protein which binds the activated PAs only 1% as tightly as PAI-1 (Kruithof, Vassalli et al. 1986). PAI-2 has been described in a disulfide-linked, inactive, higher Mᵣ form (Lecander and Åstedt 1989). PAI-2 expression appears limited to monocytes so is not normally present in serum (Wohlwend, Belin et al. 1987; Lecander and Åstedt 1989) but is relatively high in gingival tissues and fluids from periodontally diseased patients (Brown, Watanabe et al. 1995). Protease nexin-1 is a 45 kDa protein identified in association with fibroblasts which binds the PAs with less affinity than PAI-2 (Eaton, Scott et al. 1984). PAI-1, the most well characterized PAI, is also known to exist in an active and inactive state that is activatable only by denaturant treatment. It appears that PAI-1 is active when expressed on the cell surface but then released as inactive (Kooistra, Sprengers et al. 1986). tPA binds cell-associated PAI-1 and subsequently causes release of the PA/PAI complex from the cell surface (Sakata, Okada et al. 1988; Russell, Quetermous et al. 1990). PAI-1 has been shown to bind and inhibit free uPA as well as that bound to the uPAR (Cubellis, Andreasen et al. 1989; Ellis, Wun et al. 1990).

uPA, PAI-1 and the uPAR are also regulated by binding each other concomitantly. While uPA bound to uPAR can remain active for hours, when PAI-1 binds uPA, either before or after binding to the receptor, the entire complex is internalized and degraded (Nykjaer, Petersen et al. 1992; Olson, Pöllänen et al. 1992). This represents another level of activity regulation in the PA/PAI system. It is possible that this mechanism is responsible and necessary for the dynamic ECM degradation that must take place on the surface of the migrating cell.

Plasminogen may also bind the cell surface (Miles and Plow 1985; Hajjar, Harpel et al. 1986) but the binding is of low affinity and possibly mediated through non-specific interaction with glycosaminoglycans. Nevertheless, all of the PA/PAI components, including the substrate/activator plasminogen/plasmin and some MMPs, probably interact at the cell surface and provide a complex, tightly controlled, and powerful mechanism of cell-mediated ECM degradation. Further, cell-bound plasmin may be inaccessible to the serum proteinase inhibitors in some instances (Moroi and Aoki 1976; Ploug, RØnne et al. 1991).

PAs and PAIs are expressed by endothelial cells, epithelial cells, fibroblasts, PMNs and monocytes. Quiescent cells, including non-budding capillary endothelial cells, express low levels of the PAs (Bacharach, Itin et al. 1992). Normal human fibroblasts were shown to display low, discreetly localized membrane-associated levels of uPA and high levels of PAI-1 evenly on the cell surface (Pöllanen, Saksela et al. 1986). In this work, both uPA and PAI-1 were found in focal contact points together, suggesting intimate regulatory control. Levels of uPA were shown to be higher in neoplastic cell lines and increases in uPA levels have been associated with metastatic potential by other investigators, as well (O'Grady, Upfold et al. 1981; Tsuboi and Rifkin 1990; Azuma, Tamatani et al. 1993; Montgomery, De Clerck et al. 1993; Quax, de Bart et al. 1997; Takeha, Fujiyama et al. 1997).

Association of Plasminogen Activator System with Disease

Angiogenesis and vascular remodeling have been demonstrated to be highly dependent upon the expression and activation of certain proteinases and inhibitors. In some coordination with the matrix metalloproteinases (MMPs), the serine proteinase plasminogen activators, uPA (urokinase-type plasminogen activator) and tPA (tissue-type plasminogen activator), and the plasminogen activator inhibitors (PAIs), PAI-1 and PAI-2 have been strongly implicated (Mignatti and Rifkin 1996; Mazzieri, Masiero et al. 1997). Variations in either uPA, tPA, or PAI-1 expression are also demonstrated to be associated with angiogenesis and angiopathy (Gross, Mascatelli et al. 1982; Bacharach, Itin et al. 1992; Hildebrand, Dilger et al. 1995; Mignatti and Rifkin 1996). PAI-1 antigen and activity in serum, for example, is increased in two diseases associated with micro- and macrovascular pathology, namely diabetes mellitus (DM) complications and coronary artery disease (CAD) (Gray, Yudkin et al. 1992).

It was shown that the microvasculature of the gingiva accumulates peri-lumenal extracellular matrix (ECM) in association with periodontal disease severity in adults (Frantzis, Reeve et al. 1971; Listgarten, Ricker et al. 1974; Zoellner and Hunter 1994), although this has been refuted. The role of the vasculature in periodontal disease is, at this time, speculative and not fully accepted by those trained in the practice of periodontics.

However, in periodontal disease, uPA has been immunohistochemically identified in inflamed periodontal tissues (Pinchback, Gibbins et al. 1996). Intense uPA staining was associated with the blood vessels with greatest intensity in the small vessels near the epithelial pocket lining. uPA co-localized with interstitial collagenase and both uPA and collagenase stained only weakly in minimally inflamed tissues. PAs and plasmin can be measured in GCF and in gingival tissues. Plasmin activity levels and PA activity levels in both GCF and homogenized gingiva are increased in accordance with disease severity (Kaslick, Chasens et al. 1969; Hidaka, Maeda et al. 1981). Direct measurements of PAs in GCF or gingival tissues as well as inhibitor studies revealed that an increase in both tPA and uPA are associated with periodontal disease and can possibility be up to 200 fold higher concentration in GCF than in the plasma (Schmid and Chambers 1989; Brown, Watanabe et al. 1995).

Gene Polymorphisms and Genetic Testing

Each gene is represented on two alleles within the genome. Variable regions within genes, or gene polymorphisms, are represented on one or both alleles of a gene making any particular polymorphism heterozygous or homozygous, respectively. The pattern of allelic variation for each polymorphism is termed a genotype. Genetic testing is now possible (see U.S. Pat. Nos. 4,582,788 and 5,110,920) for disease associated with, or caused by, one to two genes, once the genes are identified, to determine the risk of a person carrying a given gene for the disease (see for example U.S. Pat. Nos. 4,801,531, 4,666,828, and 5,268,267).

Gene polymorphisms are often identified by their differential susceptibility to hydrolysis (commonly termed digestion) by restriction endonucleases, a large group of enzymes that hydrolyze nucleic acid polymers at specific sites relative to the contiguous nucleic acid sequence. Alternatively, polymorphisms can be identified by differential hybridization of a probe, or by polymerase chain reaction (PCR) where the size of the resulting product may vary according to the polymorphism. These methods of identifying gene polymorphisms are commonly known to those skilled in molecular biology and are available commercially, published widely in the scientific literature and on the internet.

Gene Polymorphisms in Plasminogen Activator Genes in Disease

Allelic variation (4G/5G) in the PAI-1 promoter region (Grubic, Stegnar et al. 1996) has been associated with acute coronary syndromes (Iwai, Shimoike et al. 1998) and diabetic retinopathy (Nagi, McCormack et al. 1997). A HindIII restriction fragment length polymorphism (RFLP) in the 3′ end of the PAI-1 gene has been associated with coronary artery disease (CAD) (Benza, Grenett et al. 1998), and wound healing (Benza, Grenett et al. 1998). Within the Pima Indian tribe, which presents with higher average levels of periodontal disease related to diabetes, the prevalence of a PAI-2 allelic combination within the coding region (Ser/Cys 413) which was reported as less frequent than in a control population (Foy and Grant 1997). The Alu-repeat polymorphism in intron 8 of the gene encoding for tPA (Benza, Grenett et al. 1998), and uPA gene (a BamH1 RFLP at the 3′ end) have been correlated with a greater incidence of CAD (Grenett, Reeder et al. 1998). Evidence, therefore, suggests for a role for gene polymorphisms of plasminogen activators (PA) or of PAIs in vascular disease.

This association has led others to consider certain gene polymorphisms of the plasminogen activators in periodontal disease. Of the plasminogen activator system genes, a 4G/5G promoter polymorphism in the promoter region of the gene for PAI-1 has been shown to have a significant association with periodontal disease (Izakovicova, Buckova et al. 2002).

While it is clear that the plasminogen activator system and its components have been considered in periodontal disease, and that certain gene polymorphisms within the components of the plasminogen activator system have been considered in association with periodontal disease, we remind the reader that the claims of this application consider a novel and unanticipated association of gene polymorphisms in the non-coding 3′ untranslated regions of the uPA and PAI-1 genes with chronic periodontal disease, never heretofore described.

Previous Examples

Polymorphisms in the genes encoding the immune cytokines interleukin 1 alpha and beta have been shown to be associated with severity of adult periodontitis (Kornman, Crane et al. 1997). An invention utilizing these gene polymorphisms in the genes encoding the immune cytokines interleukin 1 alpha and beta for the diagnosis of adult chronic periodontitis was protected by U.S. Pat. No. 5,686,246 (Kornman and Duff). The most significant difference between the invention described herein and that protected by U.S. Pat. No. 5,686,246 is that the means for determining gene polymorphisms associated with periodontal disease describes entirely different genes and gene polymorphisms than described herein. The invention described herein targets polymorphisms in the plasminogen activator gene uPA and its inhibitor PAI-1. Another difference between the two inventions is in the means for DNA collection described, in that a non-invasive sample collection is disclosed in the invention described here, while the invention described by U.S. Pat. No. 5,686,246 prefers the invasive collection of blood and does not clearly specify non-invasive collection means such as a buccal swab method, as described herein.

The polymorphisms in the genes encoding the immune cytokines interleukin 1 alpha and beta that were utilized in U.S. Pat. No. 5,686,246 (described above) were then protected by U.S. Pat. No. 6,130,042 (Diehl, Schenkein, and Wang) in an invention which utilizes these gene polymorphisms for the diagnosis of early onset periodontitis in children.

The polymorphisms in the genes encoding the immune cytokines interleukin 1 alpha and beta, described above, were also utilized in U.S. Pat. No. 6,524,795 (Francis, Crossman, Duff, Kornman, Stephenson) for the diagnosis of cardiovascular disorders.

A previous example of an issued patent which describes the use of plasminogen activator protein levels in oral fluids, also specifies their use in diagnosis or screening for periodontal disease (Xiao, Bunn, and Bartold, 2002; U.S. Pat. No. 6,406,873). However, importantly, this U.S. Pat. No. 6,406,873 does not consider any gene polymorphisms of the plasminogen activator system.

### SUMMARY OF THE INVENTION

According to the present invention, a method of assessing a user's predisposition to chronic periodontal disease is disclosed. The method includes the steps of isolating DNA from a user and determining gene polymorphisms (genotypes) in the 3′ untranslated regions of the genes encoding uPA and PAI-1. The identified genotypes of the user are compared to those of a control group of known periodontal disease severity, where matching the user's genotypes to the corresponding genotypes of the control sample will estimate a corresponding predisposition to chronic periodontal disease. Users determined to have genotypes associated with higher risk for periodontal disease can then maintain themselves or be professionally treated in a more aggressive manner to reduce the environmental factors which contribute to more advanced periodontal disease.

The present invention further discloses a kit for the identification of a user's genotype associated with periodontal disease severity. The kit includes DNA sample collecting means and means for determining genotypes, which are then compared to genotypes from a control group to determine the users relative risk for developing chronic periodontal disease.

### OBJECTS AND ADVANTAGES

Accordingly, the object of the disclosed invention is to provide a method for estimating a predisposition for chronic periodontal disease by assessing genomic polymorphisms in the 3′ untranslated end of the gene for uPA and for PAI-1. A further object of this invention is that it can provide a non-invasive and private manner to make this estimation of risk for developing periodontal disease, and can assist the user in choices related to health care. The advantage of this invention is that it provides a method for estimating predisposition for chronic periodontal disease which is based on genomic profile of polymorphisms in the 3′ untranslated regions of the genes encoding uPA and PAI-1, a method that is not currently available.

### DESCRIPTION OF DRAWINGS

FIG. 1: Example of a cheek scraper for collection of mucosal cells (a), of the tube in which to place the cheek scraper after collection of mucosal cells (b), and a lid to seal the collected cells within the tube until genotype determination (c).

FIG. 2: Representative sections of ethidium bromide-stained, 2% agarose gels for the PCR-based genotypic analysis for PAI-1 (HindIII), uPA, uPA; 1/1 (800 bp band), 2/2 (750 bp band), 1/2 (800 bp and 750 bp band). PAI-1 (HindIII); 1/1 (754 bp band), 2/2 (567 bp band), 1/2 (754 bp and 567 bp bands).

FIG. 2: Contingency analysis of periodontal disease severity (1-3) by the presence (−1) or absence (1) of the uPA 1/2 allele. The y-axis represents the relative probability of having a given periodontal diagnosis severity. Periodontal disease severity scores are designated in the right hand bar as mild (intermediate hatch), moderate (light hatch), and severe (dark hatch). FIG. 2a represents the entire participant cohort in the upper age group, and FIG. 2b represents the never-smoking subset of this cohort. Dimensions represent relative proportion of the total n for this analysis.

FIG. 3: Contingency analysis of the periodontal disease severity (1-3) by the PAI-1 (HindIII) genotype. The y-axis represents the relative probability of having a given periodontal disease severity (right hand bar). Periodontal disease severity scores are designated in the right hand bar as mild (intermediate hatch), moderate (light hatch), and severe (dark hatch). FIG. 3a represents the entire participant cohort in the upper age group, and FIG. 3b represents the never-smoking subset of this cohort. Dimensions represent relative proportion of the total n for this analysis.

### DETAILED DESCRIPTION OF THE INVENTION

According to the present invention, users with or without overt periodontal disease are identified as having a genetic predisposition for more severe periodontal disease by determining genotypes within the 3′ untranslated region of the gene sequence for the human gene encoding uPA (GenBank Accession no. AF377330, SEQ ID #5), and in the 3′ untranslated region of the human gene encoding PAI-1 (GenBank Accession no. AC004876, SEQ ID #6). The identified genotypes are compared to genotypes of a control group of known periodontal disease severity.

Periodontal disease is defined as set forth in the example herein and below. Briefly, periodontal disease is measured as loss of bone support for one or more teeth. Such measurements are typically made by radiographic examination, and/or by insertion of a probe along the root of a tooth with the intent to measure the extent of attachment that had been lost between the bone support and the tooth during the disease process. Such means for assessing the degree of periodontal disease are well documented and accepted by those practiced in periodontics (Armitage 1996).

Further, according to the present invention, a kit for the identification of a user's uPA and PAI-1 genotypes and their association with risk for periodontal disease is disclosed. The kit includes DNA sample collecting means and means for determining the gene polymorphisms for the uPA (BamH1) polymorphism and the PAI-1 (HindIII) polymorphism, which are then compared to data from a control group to estimate the user's predisposition for developing periodontal disease.

Source of DNA for Genotype Determination

The present invention discloses a means for collecting DNA from users. Preferably, the source of DNA for genotype determination will be from desquamating buccal mucosal cells, but this invention is not limited to that source of nuclear DNA. Preferably, the sample from the cheek scrape received from the donor will be mixed with a solution of PCR reagents for PCR-based genotype determination as described herein. An example of a cell collection device is described herein and below, however, any means for collecting DNA from a user will apply.

It may be necessary to partially extract the DNA from the cells prior to PCR. Any method well known to a person practiced in the art of molecular biology will be sufficient for this extraction of DNA. A method of DNA extraction successfully tested for this analysis is as follows:

Genomic DNA is extracted by incubating 20 μl of cell sample with 490 μl phosphate buffered saline and 1.5 ml of a 0.1% SDS solution with 250 μg proteinase K at 55° C. for 30 minutes. The DNA sample is washed twice with 500 μl phenol:chloroform:isoamyl alcohol (25:24:1), each time carefully transferring only the upper aqueous phase, then the DNA is precipitated by adding 0.1 volume 3M sodium acetate followed by 2.5 volumes of 95% cold ethanol. After centrifugation, the DNA pellet is washed with 75% cold ethanol, dried, and resuspended in Tris-EDTA for PCR.

Another, more rapid method for partially extracting DNA from scraped cheek cells, that was also successfully tested for this analysis is as follows: the cheek was scraped for several seconds and the sample was stirred in 50 μl of 200 mM NaOH, then boiled for 10 minutes, cooled at room temperature, and neutralized with 50 μl of 100 mM HCl with 100 mM Tris HCL-base, pH 8.5. PCR was performed as below using 2 μl of this DNA sample.

Means for Genotype Determination

The present invention discloses a means for genotype determination. Preferably, genotype determination will be made by PCR followed by restriction endonuclease digestion, although any other method known to those skilled in molecular biology may be used, such as Southern blot analysis. Examples of methods utilized for determining gene polymorphisms are described in U.S. Pat. No. 5,858,659 and U.S. Pat. No. 5,856,104.

PCR may be performed by any acceptable method and conditions. A common example of standard PCR methodology and conditions is as follows: in a reaction buffer of 20 mM Tris-HCl (pH 8.4), 50 mM KCl, 200 mM dATP, dTTP, dGTP, dCTP each, 1.0 mM MgCl₂ and the DNA provided by the cells of the cheek scrape, 1.0 unit of Taq polymerase will be added. A typical PCR cycle is performed with 32 cycles of 95° C., 60° C., and 72° C. for 20 s, 60 s, and 30 s, respectively. An aliquot of the PCR reaction could be analyzed on a horizontal slab agarose gel.

A PCR protocol that has been successfully tested for particular uPA and PAI-1 genotype determinations is as follows: PCR was performed with approximately 70 ng sample DNA using a commercially prepared mix of buffer, polymerase, and dNTPs (Platinum PCR Supermix, Invitrogen Corp PCR was performed with the addition of 12.5 pmol of forward and reverse primers as described previously for the BamH1 uPA polymorphism (Sell, Blasi et al. 2000), and the PAI-1 (HindIII) polymorphism (Grenett, Khan et al. 2000). Samples were initially denatured at 94° C. for 5 minutes, then cycled 36 times as follows: 94° C. for 45 sec, 55° C. for 40 sec; 72° C. for 45 sec. After a final extension at 72° C. for 2 minutes, the samples were cooled then prepared for restriction digestion as indicated.

The gene polymorphisms for uPA (BamH1) and PAI-1 (HindIII) can be identified by allele-specific cleavage using a restriction enzyme. After restriction endonuclease digestion, the genotype of each sample would be identified as one of three distinct DNA fragment patterns produced after digestion with either HindIII restriction enzyme for PAI-1, or with BamH1 restriction enzyme for uPA. These restriction endonuclease enzymes are commonly available commercially. The genotype (one of three distinct DNA fragment patterns) would be determined by visualization of the digested PCR products within an agarose gel using ethidium bromide to identify the DNA in ultraviolet light. DNA fragment sizes are typically analyzed by gel electrophoresis, where mobility in the gel under electric current is proportional to fragment size. However, other means of determining the gene polymorphism in the designated regions of the uPA and PAI-1 gene also apply.

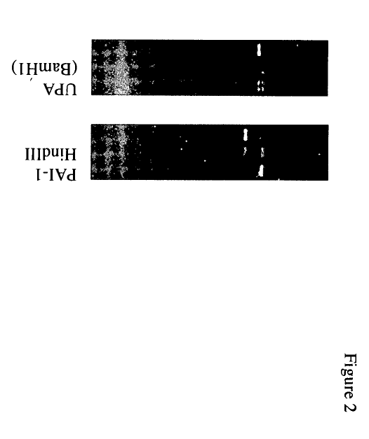

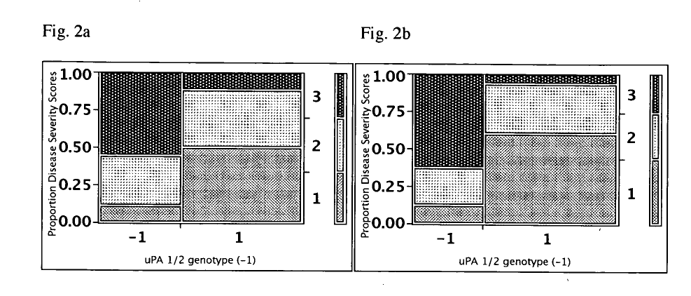

For digestion, 10 μl of PCR sample could be directly added to 15 μl of the appropriate digest buffer and enzyme (Table 1), then incubated at 37° C. overnight. Digested samples would be mixed with loading buffer and products were separated on a 2% agarose gel in TBE. Non-digested PCR product for each reaction type would be included in each analysis for control. Restriction enzymes could be obtained commercially. After electrophoretic separation of the products, the gels would be stained with 0.001% ethidium bromide in water. Products of this preferred protocol are represented in FIG. 2. Preferable primers for determining these genomic polymorphisms are listed below in Table 1, although any primers which amplify the region of the genomic polymorphism of interest will suffice and apply.

TABLE 1
Preferred primer pairs and restriction enzyme for PCR-based
determination of genotypes

| | | | Restriction |
|---|---|---|---|
| Reaction | SEQ ID NO: | Primers | Enzyme |
| uPA | 1 | Forward - GCCTAGTTCATCCAATCCTC | BamH1 |
| | 2 | Reverse - AGTGCAGTGGCACAATCAGC | |
| PAI-1 | 3 | Forward - GCCTCCAGCTACCGTTATTGTACA | HindIII |
| | 4 | Reverse - CAGCCTAAACAACAGAGACCCC | |

Specific polymorphisms in DNA sequences coding for uPA and PAI-1 were found to be associated with severity of chronic periodontal disease. The polymorphisms are as follows:
uPA: (Gene name PLAU, gene ID 5328, Chromosome 10 at 10q24, Gene Accession # AF377330)
   The gene polymorphism on one or both alleles (T/G) at base 8121 lying within the 3′ untranslated region of the uPA gene (SEQ ID #5) is identified using a restriction endonuclease named BamH1. The polymorphism is located within the BamH1 site (sequence GGATCC) surrounding base 8121, where one allele is hydrolyzed by BamH1 activity and the other allele is not, thereby producing differential restriction enzyme digestion fragments of the genomic DNA in this gene region. This polymorphism lies outside of the protein coding sequence (bases 2207-3055) in the 3′ untranslated region of the uPA gene (bases 3056-10075 of the gene).
PAI-1 (Gene name SERPINE1, gene ID 5054, Chromosome 7 at 7q21.3-q22, gene accession # AC004876)

The gene polymorphism on one or both alleles lying within the 3′ untranslated region of the PAI-1 gene (SEQ ID #6) is identified using a restriction endonuclease named HindIII. The polymorphism is located within the HindIII site (sequence AAGCTT) within bases 37873-37878, where one allele is hydrolyzed by HindIII activity and the other allele is not, thereby producing differential restriction enzyme digestion fragments of the genomic DNA in this gene region. This polymorphism lies outside of the protein coding sequence (bases 23159-34338) in the 3′ untranslated region of the PAI-1 gene (bases 34339-126462 of the gene).

The user's genotypes for uPA and PAI-1 are then compared to genotypes of controls. The controls are from a group of individuals of a known and wide range of chronic periodontal disease severity, and their genotypes will have been statistically associated with a given periodontal disease status. Matching the user's genotype to the corresponding genotype of the control sample will thereby estimate a corresponding predisposition to chronic periodontal disease.

Prophetic DNA Collection Device

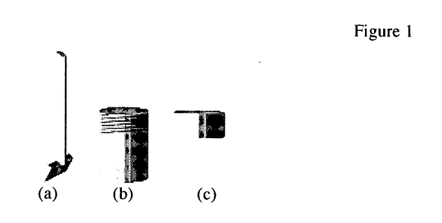

Preferably, the invention will utilize DNA samples collected by a kit which will allow safe and sterile collection of the samples and maintain them until genotype determination is made. As an example of such a kit, each kit could contain a sterile device to non-invasively wipe the lining of the cheek in the mouth such as a plastic cheek scraper on a plastic handle (2-6 cm in length) (See FIG. 1a). Each kit could also contain a polycarbonate container (5-7 cm long) with lid (FIGS. 1b and 1c), instructions for use, a small return mailer with the laboratory address and postage, and a return address data sheet for the user. The cheek scraper could be supplied in a sealed sterile plastic bag or the tube.

To demonstrate usefulness and simplicity, an example of user instructions would be similar to the following.
1) Complete the Return Address Data Sheet and place in the enclosed pre-addressed mailer. This is the address (postal or email) and name to which you wish the results to be returned.
2) Open one sealed cheek swab at the end of the package designated “OPEN HERE” and remove the sterile cheek scraper. IMPORTANT—TOUCH ONLY THE HANDLE AND DO NOT TOUCH THE BROAD END OF THE SCRAPER, as this may affect the validity of the results.
3) Gently scrape the inside lining of your right cheek five times, then your left cheek five times.
4) Place the scraper back into the tube and seal the tube with the lid.
5) Repeat steps 2-4 with the second sampler.
6) Place both tightly sealed containers in the pre-addressed mailer with the return address information, seal the mailer, and place in the mailbox.
7) Your results will be returned to the address you requested (postal or email) within 1-2 weeks.

A kit such as this could be used to collect samples from the tongue or another mucosal surface, the intent being to obtain DNA from the user. Such samples could also include the collection of mucous secretions such as saliva, as an example.

The above discussion provides a factual basis for a kit for the determination of a user's uPA or PAI-1 genotype and a corresponding predisposition to chronic periodontal disease.

### OPERATION OF INVENTION

The methods used with the present invention, and the utility of the present invention, can be shown by the following example. A convenient sample of patients presenting for treatment to University Health Center was recruited over the course of two years with informed consent. A score for periodontal disease severity was determined by radiographic examination for each participant by one investigator using American Dental Association case type categories for periodontal diagnosis (none, mild, moderate, and severe). In determining a score for periodontal disease severity, teeth were assigned a periodontal disease designation of none, mild, moderate, or severe based on the greatest extent of alveolar bone loss for each tooth, where the designations of mild represented <25% crestal bone loss, moderate represented 25%-50% crestal bone loss, and severe represented >50% crestal bone loss, relative to the distance from the apex to a point 2 mm apical to the cemento-enamel junction. The diagnosis of mild periodontal disease had the criterion that at least one site demonstrated radiographic evidence of at least, but no more than, mild crestal alveolar bone loss. The diagnosis of moderate had the criterion that at least one site had a designation of moderate and demonstrated between 25% and 50% crestal bone loss but had no teeth with a designation of severe. The diagnosis of severe periodontal disease had the criterion that at least one site was designated as severe and demonstrated >50% crestal alveolar bone loss.

Smoking history (yes/no), and diabetes status (none or type 2) were obtained verbally, and HgA1c values were used to corroborate diabetes status. After obtaining informed consent and verbal history, venous blood was collected from the participants, and genomic DNA was extracted from peripheral blood cells. PCR was performed with the addition of 12.5 pmol of forward and reverse primers as described previously for the BamH1 uPA polymorphism (Sell, Blasi et al. 2000), and the PAI-1 (HindIII) polymorphism (Grenett, Khan et al. 2000) (see Table 1). PCR products were digested with restriction digest enzymes as indicated in Table 1.

Genotypes for uPA, and PAI-1 (HindIII) were determined for each participant from the stained gels (FIG. 2). Considering the ordinal designations of periodontal disease severity, analyses were performed by ordinal logistic regression. Where indicated, prediction of the dependent outcome, periodontal disease severity, was modeled with the inclusion of periodontal disease risk factors as covariates, including age, smoking (yes/no), and diabetes status (none, or type 2).

In an analysis of all of the participants above the median age in our population (range 66-83, 55% female), including smokers (79% non-Hispanic white, 10% Hispanic, 6% Black, and 4% Asian; n=50), there was a significant association of the 1/2 uPA allele with periodontal disease severity, which ranged from mild to severe disease (32% mild, 38%, moderate, and 28% with severe periodontal disease). In this cohort, neither age, smoking history, nor diabetes status were, independently, significant predictors of periodontal disease status (p>0.05). However, the presence of the 1/2 heterozygous uPA allele was, by itself, a significant predictor of more severe periodontal disease (χ²=11.4, p=0.0007 (FIG. 2a). With the inclusion of smoking history, age, and diabetes status as risk factors in the model, the adjusted odds ratio for the uPA 1/2 allele effect on periodontal disease severity within the model was 2.8 (p=0.002), and was the only independent variable with statistical significance.

In our cohort of participants over 56 years old with no smoking history (68% non-Hispanic white, 12% Black, 12% Hispanic, and 8% Asian; n=25, range 56-79 years, 50% female), the distribution of periodontal disease severity ranged from mild to severe disease (40% mild, 36% moderate, and 24% with severe periodontal disease). In this never-smoking group, the periodontal diagnosis severity was not significantly associated with diabetes status nor with age (p>0.05). However, in subjects analyzed for their uPA genotype, the presence of the 1/2 heterozygous uPA allele, by itself, was significantly associated with more severe periodontal disease (odds ratio of 3.7, χ²=9.4, p=0.006) (FIG. 2b). In a model with diabetes status and age included as covariates in the analysis for predicting periodontal disease severity, the adjusted odds ratio for the presence of the 1/2 uPA allele within the model was improved (odds ratio=4.3, p=0.005), while neither age nor diabetes status were significant (p>0.05).

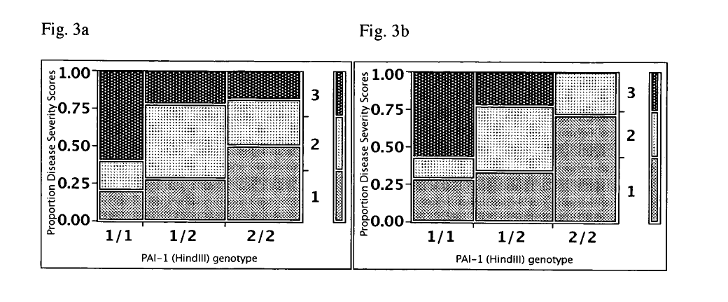

The PAI-1 HindIII genotype also predicted periodontal disease severity. Sampling from our participants above the median age, including smokers, the presence of the “1” allele was directly associated with periodontal disease severity (FIG. 3a). In a model controlling for age, smoking history, and diabetes status, the presence of the homozygous 1/1 PAI-1 allele had an adjusted odds ratio of 14.8 within the model relative to the presence of the 2/2 PAI-1 allele (p=0.03) for the prediction of periodontal disease severity. In this model, the effects of smoking history, age, and diabetes status were insignificant.

In the never-smoking cohort, the “1” allele of the PAI-1 HindIII genotype also demonstrated a direct relationship with periodontal disease severity (FIG. 3b). When controlling for age and diabetes status, the presence of the heterozygous PAI-1 (HindIII) 1/2 allele was predictive of disease with an odds ratio of 9.5 compared to participants with an absence of a “1” allele (p=0.05). However, the presence of the homozygous 1/1 allele for the PAI-1 (HindIII) polymorphism did not significantly improve disease prediction in this model relative to the heterozygous allele. In this model, the effects of smoking history, age, and diabetes status were also insignificant. A model which combined the 1/2 uPA allele with distribution of the PAI-1 “1” allele polymorphism, strongly predicted periodontal disease severity with a χ2=13.7 (p=0.003) within the never-smoking cohort of participants above 56 years of age. Comparatively, a competing model, utilizing as the sole independent variables age and diabetes status, was poor for the prediction of periodontal disease severity in this cohort (χ²=0.76) and not significant. In a model which combined age and diabetes status together with these uPA and PAI-1 genotypes for the prediction of periodontal disease severity, the whole model fit (χ²=16.7, p=0.005) was slightly better than represented by the uPA and PAI-1 genotypes alone.

In an analysis of all the participants, including those with a smoking history, the abilities of age, diabetes status, and smoking history to predict periodontal disease severity were not significant as a model. However, when the PAI-1 (HindIII) “1” allele and the uPA 1/2 allele were added as independent variables to the model, we were able to significantly predict periodontal disease severity in these same participants (χ²=24.4, p=0.0004). In a comparative model, with the independent variables limited to just the 1/2 uPA allele and distribution of the PAI-1 “1” allele polymorphism, prediction of periodontal disease severity was still highly significant (χ²=15.9, p=0.001). These analyses demonstrated a highly significant contribution of the combined uPA and PAI-1 (HindIII) genotype in the prediction of periodontal disease severity within older adults.

Asian participants had a significantly lower prevalence of the uPA 1/2 allele (p=0.03). The homozygous 1/1 PAI-1 HindIII genotype was significantly more prevalent in the Black and Hispanic participants than in the non-Hispanic white or Asian participants (p=0.02). There was not a significant relationship, however, (p>0.05) between gender and any of the genotypes that were analyzed.

We have limited our analysis to older participants, using the top age median for each analysis. This limitation is useful in the cross-sectional analyses of periodontal disease severity and other chronic disease analysis. In an older cohort, disease in those inherently more susceptible will have had more time to advance, while in those inherently more resistant, periodontal disease will have advanced more slowly relative to the more susceptible group, on average.

Age, diabetes status, and smoking are well known risk factors for periodontal disease, and were included in prediction analyses as described (for a review of periodontal disease and related risk factors see (Fenesy 1998)). Because smoking is so strong a risk factor for periodontal disease, our participant cohorts were analyzed with, and without, the inclusion of smokers to provide an indication of effect-strength of the uPA and PAI-1 genotypes relative to periodontal disease severity. The individual effects of the uPA and PAI-1 genotypes in the various models predicting periodontal disease severity were naturally lowered when smokers were included and the smoking effect was partitioned into the model. However, the remaining adjusted odds ratios estimated for the uPA and PAI-1 genotypes in these cohorts remained statistically significant.

Unrelated to the plasminogen activators and their inhibitors, a polymorphism in the IL-1β gene has been associated with severity of periodontal disease (Kornman, Crane et al. 1997) and is commercially available as a diagnostic test. This genotype analysis has provided some practitioners and patients with a helpful adjunct in treatment planning and case management. Other genomic polymorphisms of immune molecules have been examined for interaction with periodontal disease severity, and one, pertaining to the Fc-gamma receptor, may also be significant (for a review of genomic considerations in periodontal disease see (Kinane and Hart 2003)). With the relatively high and significant odds ratios for disease severity provided by the uPA and PAI-1 (HindIII) genotypes, it is possible these genomic markers will also provide support to clinicians and patients in oral health treatment planning.

The combined uPA (BamH1) and PAI-1 (HindIII) genotypes, related to the plasminogen activators, demonstrated a novel and highly significant model for the prediction of periodontal disease severity within our sample population. This combined model resulted in greater fit and significance than was provided by either genotype alone. In limiting our analyses to the older cohort, these uPA and PAI-1 genotypes were uniquely predictive of periodontal status.

### DESCRIPTION AND OPERATION OF ALTERNATIVE EMBODIMENTS

Not applicable.

### CONCLUSION, RAMIFICATIONS, AND SCOPE OF INVENTION

Thus the reader will see that a novel and unanticipated means for estimating predisposition for chronic periodontal disease by identifying polymorphisms within the 3′ untranslated end of the uPA gene and/or the PAI-1 gene, and comparing said gene polymorphisms to those of a control group with know periodontal disease status, provides the methods for a useful, non-invasive, economical adjunct in health care for the majority of the population. While the above description contains many methodological specificities, these should not be construed as limitations on the scope of the invention, but rather as an exemplification of one preferred embodiment thereof. Many other methodological variations are possible, some suggested within the specification. Accordingly, the scope of the invention should be determined not by the embodiments illustrated, but by the appended claims and their legal equivalents.

### REFERENCES

The following references were cited in the above disclosure or relied on therein and are incorporated by reference in their entirety.
Appella, E., E. A. Robinson, et al. (1987). “The receptor-binding sequence of urokinase. A biological function for the growth factor module of proteases.” Journal of Biological Chemistry 262: 4437-4440.
Armitage, G. C. (1996). “Periodontal diseases: diagnosis.” Ann Periodontol 1(1): 37-215.
Azuma, M., T. Tamatani, et al. (1993). “Role of plasminogen activators, metalloproteinases and the tissue inhibitor of metalloproteinase-1 in the metastatic process of human salivary-gland adenocarcinoma cells.” International Journal of Cancer 54(4): 669-76.
Bacharach, E., A. Itin, et al. (1992). “In vivo patterns of expression of urokinase and its inhibitor PAI-1 suggest a concerted role in regulating physiological angiogenesis.” Proceedings of the National Academy of Sciences 89: 10686-10690.
Beck, J. D., S. Offenbacher, et al. (1998). “Periodontitis: a risk factor for coronary heart disease?” Annals of Periodontology 3(1): 127-41.
Benza, R. L., H. Grenett, et al. (1998). “Gene polymorphisms for PAI-1 are associated with angiographic extent of coronary artery disease.” Journal of Thrombosis and Thrombolysis 5: 143-150.
Benza, R. L., H. E. Grenett, et al. (1998). “Gene polymorphisms for plasminogen activator inhibitor-1/tissue plasminogen activator and development of allograft coronary artery disease.” Circulation 98(21): 2248-2254.
Brown, J. M., K. Watanabe, et al. (1995). “Molecular characterization of plasminogen activators in human gingival crevicular fluid.” Archives of Oral Biology 40(9): 839-845.
Cubellis, M. V., P. Andreasen, et al. (1989). “Accessibility of receptor-bound urokinase to type-1 plasminogen activator inhibitor.” Proceedings of the National Academy of Sciences 86: 4828-4832.
Eaton, D. L., R. W. Scott, et al. (1984). “Purification of human fibroblast urokinase proenzyme and analysis of its regulation by proteases and protease nexin.” Journal of Biological Chemistry 259: 6241-6247.
Egelberg, J. (1966). “The blood vessels of the dento-gingival junction.” Journal of Periodontal Research 1: 163-179.
Ellis, V., T.-C. Wun, et al. (1990). “Inhibition of receptor-bound urokinase by plasminogen-activator inhibitors.” Journal of Biological Chemistry 265(17): 9904-9908.
Fenesy, K. E. (1998). “Periodontal disease: an overview for physicians.” The Mount Sinai Journal of Medicine 65(5-6): 362-369.
Foy, C. A. and P. J. Grant (1997). “PCR-RFLP detection of PAI-2 gene variants: prevalence in ethnic groups and disease relationship in patients undergoing coronary angiography.” Thrombosis and Haemostasis 77(5): 955-958.
Frantzis, T. G., C. M. Reeve, et al. (1971). “The ultrastructure of capillary basement membranes in the attached gingiva of diabetic and nondiabetic patients with periodontal disease.” Journal of Periodontology 42: 406-411.
Gray, R. P., J. S. Yudkin, et al. (1992). “Plasminogen activator inhibitor: a risk factor for myocardial infarction in diabetic patients.” British Heart Journal 69: 228-232.
Grenett, H. E., N. Khan, et al. (2000). “Identification of the HindIII polymorphic site in the PAI-1 gene: analysis of the PAI-1 HindIII polymorphism by PCR.” Genetic Testing 4(1): 65-68.
Grenett, H. E., V. C. Reeder, et al. (1998). “Identification of a BamHI polymorphism for the urokinase gene associated with symptomatic coronary artery disease.” Journal of Thrombosis and Thrombolysis 5: 113-118.
Gross, J. L., D. Mascatelli, et al. (1982). “Plasminogen activator and collagenase production by cultured endothelial cells.” Journal of Cell Biology 95: 974-981.
Grossi, S. G., F. B. Skrepcinski, et al. (1997). “Treatment of periodontal disease in diabetics reduces glycated hemoglobin.” Journal of Periodontology 68(8): 713-719.
Grubic, N., M. Stegnar, et al. (1996). “A novel G/A and the 4G/5G polymorphism within the promoter of the plasminogen activator inhibitor-1 gene in patients with deep vein thrombosis.” Thrombosis Research 84: 431-443.
Haffajee, A. D., S. S. Socransky, et al. (1988). “Clinical, microbiological and immunological features of subjects with destructive periodontal diseases.” Journal of Clinical Periodontology 15: 240-246.
Hajjar, K. A., P. C. Harpel, et al. (1986). “Binding of plasminogen to cultured human endothelial cells.” Journal of Biological Chemistry 261(11656-11662).
Hidaka, N., K. Maeda, et al. (1981). “Fibrinolytic activity in periodontal disease. The relationship between fibrinolytic activity and severity of periodontal disease.” Journal of Periodontology 52(4): 181-186.
Hildebrand, R., I. Dilger, et al. (1995). “Urokinase and macrophages in tumour angiogenesis.” British Journal of Cancer 72(4): 818-823.
Iwai, N., H. Shimoike, et al. (1998). “The 4G/5G polymorphism of the plasminogen activator inhibitor gene is associated with the time course of progression to acute coronary syndromes.” Atherosclerosis 136(1): 109-114.
Izakovicova, H. L., D. Buckova, et al. (2002). “Plasminogen-activator-inhibitor-1 promoter polymorphism as a risk factor for adult periodontitis in non-smokers.” Genes and Immunity 3(5): 292-294.
Kaslick, R. S., A. I. Chasens, et al. (1969). “Tissue plasminogen in normal and inflamed human gingiva.” Journal of Dental Research 48(4): 603.
Kinane, D. F. and T. C. Hart (2003). “Genes and gene polymorphisms associated with periodontal disease.” Critical Reviews in Oral Biology and Medicine 14(6): 430-449.
Kooistra, T., E. D. Sprengers, et al. (1986). “Rapid inactivation of the plasminogen activator-inhibitor upon secretion from cultured human endothelial cells.” Biochemical Journal 239: 497-503.
Kornman, K. S., A. Crane, et al. (1997). “The interleukin-1 genotype as a severity factor in adult periodontal disease.” Journal of Clinical Periodontology 24: 72-77.
Kroon, M. E., P. Koolwijk, et al. (1999). “Role and localization of urokinase receptor in the formation of new microvascular structures in fibrin matrices.” American Journal of Pathology 154(6): 1731-1742.
Kruithof, E. K., J. D. Vassalli, et al. (1986). “Purification and characterization of a plasminogen activator inhibitor from the histiocytic lymphoma cell line U-937.” Journal of Biological Chemistry 261: 11207-11213.
Lecander, I. and B. Åstedt (1989). “Occurence of a specific plasminogen activator inhibitor of placental type PAI-2, in men and non-pregnant women.” Fibrinolysis 3: 27-30.
Listgarten, M. A., F. H. Ricker, et al. (1974). “Vascular basement lamina thickness in the normal and inflamed gingiva of diabetics and non-diabetics.” Journal of Periodontology 45: 676-301.
Loe, H., A. Anerud, et al. (1986). “Natural history of periodontal disease in man. Rapid, moderate and no loss of attachment in Sri Lankan laborers 14 to 46 years of age.” J Clin Periodontol 13(5): 431-45.
Longstaff, C., R. E. Merton, et al. (1999). “Characterization of cell-associated plasminogen activation catalyzed by urokinase-type plasminogen activator, but independent of urokinase receptor (uPAR, CD87).” Blood 93(11): 3839-3846.
Mazzieri, R., L. Masiero, et al. (1997). “Control of type IV collagenase activity by components of the urokinase-plasmin system: a regulatory mechanism with cell-bound reactants.” EMBO 16(9): 2319-2332.
Mignatti, P. and D. B. Rifkin (1993). “Biology and biochemistry of proteinases in tumor invasion.” Physiological Reviews 73(1): 161-195.
Mignatti, P. and D. B. Rifkin (1996). “Plasminogen activators and matrix metalloproteinases in angiogenesis.” Enzyme and Protein 49: 117-137.
Miles, L. A. and E. F. Plow (1985). “Binding and activation of plasminogen on the platelet surface.” Journal of Biological Chemistry 260: 4303-4311.
Montgomery, A. M., Y. A. De Clerck, et al. (1993). “Melanoma-mediated dissolution of extracellular matrix: contribution of urokinase-dependent and metalloproteinase-dependent proteolytic pathways.” Cancer Research 53: 693-700.
Moroi, M. and N. Aoki (1976). “Isolation and characterization of alpha2-plasmin inhibitor from human plasma. A novel proteinase inhibitor which inhibits activator-induced clot lysis.” Journal of Biological Chemistry 251: 5956-5965.
Nagi, D. K., L. J. McCormack, et al. (1997). “Diabetic retinopathy, promoter (4G/5G) polymorphism of PAI-1 gene, and PAI-1 activity in Pima Indians with type 2 diabetes.” Diabetes Care 20(8): 1304-1309.
Nykjaer, A., C. M. Petersen, et al. (1992). “Purified a2-macroglobulin receptor/LDL receptor-related protein binds urokinase-plasminogen activator inhibitor type-1 complex.” Journal of Biological Chemistry 267(21): 14543-14546.
O'Grady, R. L., L. I. Upfold, et al. (1981). “Rat mammary carcinoma cells secrete active collagenase and activate latent enzyme in the stroma via plasminogen activator.” Int. J. Cancer 28: 509-515.
Offenbacher, S., J. D. Beck, et al. (1998). “Role of periodontitis in systemic health: spontaneous preterm birth.” Journal of Dental Education 62(10): 852-858.
Oliver, R. C., L. J. Brown, et al. (1989). “An estimate of periodontal treatment needs in the U.S. based on epidemilogic data.” Journal of Periodontology 60(7): 371-380.
Olson, D., J. Pöllänen, et al. (1992). “Internalization of the urokinase-plasminogen activator inhibitor type-1 complex is mediated by the urokinase receptor.” Journal of Biological Chemistry 267(13): 9129-9133.
Petersen, L. C., L. R. Lund, et al. (1988). “One chain urokinase-type plasminogen activator from human sarcoma cells is a proenzyme with little or no intrinsic activity.” Journal of Biological Chemistry 263: 11189-11195.
Pinchback, J. S., J. R. Gibbins, et al. (1996). “Vascular co-localization of proteolytic enzymes and proteinase inhibitors in advanced periodontitis.” Journal of Pathology 179: 326-332.
Pinchback, J. S., B. A. Taylor, et al. (1996). “Microvascular angiopathy in advanced periodontal disease.” Journal of Pathology 179: 204-209.
Ploug, M., E. RØnne, et al. (1991). “Cellular receptor for urokinase plasminogen activator. Carboxy-terminal processing and membrane anchoring by glycosyl-phosphatidylinositol.” Journal of Biological Chemistry 266: 1926-1933.
Pöllanen, J., O. Saksela, et al. (1986). “Distinct localizations of urokinase-type plasminogen activator and its type I inhibitor under cultured human fibroblasts and sarcoma cells.” Journal of Cell Biology 104: 1085-1096.
Quax, P. H., A. C. de Bart, et al. (1997). “Plasminogen activator and matrix metalloproteinase production and extracellular matrix degradation by rat prostate cancer cells in vitro: correlation with metastatic behavior in vivo.” The Prostate 32: 196-204.
Rolden, A. L., M. V. Cubellis, et al. (1990). “Cloning and expression of the receptor for human urokinase plasminogen activator, a central molecule in cell surface, plasmin dependent proteolysis.” EMBO 9: 467-474.
Russell, M. E., T. Quetermous, et al. (1990). “Binding of tissue-type plasminogen activator with human endothelial cell monolayers. Characterization of the high affinity interaction with plasminogen activator inhibitor-1.” Journal of Biological Chemistry 265(5): 2569-2575.
Sakata, Y., M. Okada, et al. (1988). “Interaction of tissue-type plasminogen activator and plasminogen activator inhibitor 1 on the surface of endothelial cells.” Journal of Biological Chemistry 263(4): 1960-1969.
Schmid, J. and D. A. Chambers (1989). “Molecular characterization of plasminogen activator in human supragingival plaque.” Archives of Oral Biology 34(1): 17-21.
Sell, S. M., F. Blasi, et al. (2000). “Automated, PCR-RFLP genotyping of the urokinase gene.” Genetic Testing 4(3): 305-307.
Takeha, S., Y. Fujiyama, et al. (1997). “Stromal expression of MMP-9 and urokinase receptor is inversely associated with liver metastasis and with infiltrating growth in human colorectal cancer: a novel approach from immune/inflammatory aspect.” Japanese Journal of Cancer Research 88: 72-81.
Tsuboi, R. and D. B. Rifkin (1990). “Bimodal relationship between invasion of the amniotic membrane and plasminogen activator activity.” International Journal of Cancer 46(1): 56-60.
Vassalli, J. D., D. Baccino, et al. (1985). “A cellular binding site for the Mᵣ 55,000 form of the human plasminogen activator, urokinase.” Journal of Cell Biology 100: 86-92.
Wohlwend, A., D. Belin, et al. (1987). “Plasminogen activator-specific inhibitors produced by human monocytes/macrophages.” Journal of Experimental Medicine 165: 321-339.
Zoellner, H. and N. Hunter (1991). “Vascular expansion in chronic periodontitis.” Journal of Oral Pathology and Medicine 20: 433-437.
Zoellner, H. and N. Hunter (1994). “The vascular response in chronic periodontitis.” Australian Dental Journal 39(2): 93-97.

We claim:

1) A method of predicting a user's susceptibility to periodontal disease, comprising the steps of:
a) isolating genomic DNA from a patient;
b) determining the genotype for the 3′ untranslated region of uPA gene in the genomic DNA (SEQ ID #5); and
c) comparing the genotype to a control sample of
a. known genotypes for the 3′ untranslated region of uPA gene in the genomic DNA, and
b. known periodontal disease status
wherein said control sample comprises three different genotypes that are statistically associated with a given periodontal disease status, and wherein the similarity of the user's genotype to the control sample indicates a corresponding susceptibility to periodontal disease.

2) The method as set forth in claim 1 wherein said step for determining in the DNA a genotype for uPA includes amplification of target DNA sequences using the polymerase chain reaction (PCR) wherein the PCR primers used are:

| | |
|---|---|
| 5′ GCCTAGTTCATCCAATCCTC 3′ | SEQ ID NO: 1 |
| 5′ AGTGCAGTGGCACAATCAGC 3′ | SEQ ID NO: 2 |

3) The method as set forth in claim 1 wherein said step for determining in the DNA a genotype for uPA includes restriction endonuclease enzyme digestion with restriction endonuclease enzyme BamH1.

4) A method of predicting a user's susceptibility to periodontal disease, comprising the steps of:
(a) isolating genomic DNA from a patient;
(b) determining the genotype for the 3′ untranslated region of the PAI-1 gene in the genomic DNA (SEQ ID #6); and
(c) comparing the genotype to a control sample of
a. known genotypes for the 3′ untranslated region of the PAI-1 gene in the genomic DNA, and
b. known periodontal disease status
wherein said control sample comprises three different genotypes that are statistically associated with a given periodontal disease status, and wherein the similarity of the user's genotype to the control sample indicates a corresponding susceptibility to periodontal disease.

5) The method as set forth in claim 4 wherein said step for determining in the DNA a genotype for PAI-1 includes amplification of target DNA sequences using the polymerase chain reaction (PCR) wherein the PCR primers used are:

| | |
|---|---|
| 5′ GCCTCCAGCTACCGTTATTGTACA 3′ | SEQ ID NO: 3 |
| 5′ CAGCCTAAACAACAGAGACCCC 3′ | SEQ ID NO: 4 |

6) The method as set forth in claim 4 wherein said step for determining in the DNA a genotype for PAI-1 includes restriction endonuclease enzyme digestion with restriction endonuclease enzyme HindIII.

7) A method of predicting a user's susceptibility to periodontal disease, comprising the steps of:
(a) isolating genomic DNA from a patient;
(b) determining the genotypes for the 3′ untranslated regions of the uPA gene (SEQ ID #5) and the PAI-1 gene (SEQ ID #6) in the genomic DNA; and
(c) comparing the genotypes to a control sample of
a. known genotypes for the 3′ untranslated regions of the uPA gene and the PAI-1 gene in the genomic DNA, and
b. known periodontal disease status
wherein said control sample comprises three different genotypes in the uPA gene and three different genotypes in the PAI-1 gene that are statistically associated with a given periodontal disease status, and wherein the similarity of the user's genotypes to the control sample indicates a corresponding susceptibility to periodontal disease.

8) The method as set forth in claim 7 wherein said step for determining in the DNA genotypes for uPA and PAI-1 includes amplification of target DNA sequences using the polymerase chain reaction (PCR) wherein the PCR primers used are:

| | |
|---|---|
| 5′ GCCTAGTTCATCCAATCCTC 3′ | SEQ ID NO: 1 |
| 5′ AGTGCAGTGGCACAATCAGC 3′ | SEQ ID NO: 2 |
| 5′ GCCTCCAGCTACCGTTATTGTACA 3′ | SEQ ID NO: 3 |
| 5′ CAGCCTAAACAACAGAGACCCC 3′ | SEQ ID NO: 4 |

9) The method as set forth in claim 7 wherein said step for determining in the DNA genotypes for uPA and PAI-1 includes restriction endonuclease enzyme digestion with restriction endonuclease enzymes BamH1 and HindIII.

10) A kit for predicting a user's susceptibility to periodontal disease, said kit comprising:
(a) a DNA sample collecting means
(b) a means for determining genotypes within the 3′ untranslated regions of the genes encoding uPA and PAI-1; and
(c) a control sample comprising known genotypes for uPA and PAI and known periodontal disease status.

11) The kit as set forth in claim 10 wherein said kit comprises a set of polymerase chain reaction (PCR) primers, wherein said primers consist of:

| | |
|---|---|
| 5′ GCCTAGTTCATCCAATCCTC 3′ | SEQ ID NO: 1 |
| 5′ AGTGCAGTGGCACAATCAGC 3′ | SEQ ID NO: 2 |
| 5′ GCCTCCAGCTACCGTTATTGTACA 3′ | SEQ ID NO: 3 |
| 5′ CAGCCTAAACAACAGAGACCCC 3′ | SEQ ID NO: 4 |


SEQUENCE LISTING (6 sequences)

SEQ ID NO: 1 - DNA, 20 bp, Homo sapiens
gcctagttcatccaatcctc

SEQ ID NO: 2 - DNA, 20 bp, Homo sapiens
agtgcagtggcacaatcagc

SEQ ID NO: 3 - DNA, 24 bp, Homo sapiens
gcctccagctaccgttattgtaca

SEQ ID NO: 4 - DNA, 22 bp, Homo sapiens
cagcctaaacaacagagacccc

SEQ ID NO: 5 - DNA, 10075 bp, Homo sapiens
taatgggttctcaaatccaaacaaaactgaccacagtcttctagaggaagtagcaaggttggctctgaagcctatagcatctctgactcagtctgtcccctggaaggctggcagctcagcaagcacagaagtctctccagaagacagtgggtcacctgcctcccaaaagctgaaaggctaacttgtactttccccagcaggcagctggcatcctgagccctcggctggggcagagcaaaggagccttcctccttcctaccttcctggcactctccctgccttccttctgtcactctcaggtggacccagacccaaggtccagatttgcaaggcaggaaaatgctgcaggcctaggctgggaaagggcccaaagccgctagtggattgctgggactcagcctcctccttcccactaagagagcgagtcctactgggttcaaaatgaccccaagccctggttcctgacactaggggaaagagatgggggtgacagaatcacagaatccctgctatgttcctccaagtgtgcccagagatgcgtgtgtgtgtgtgtgtgtatacacaaatgtctgcttatcctcaggcaggaagggtggatgcagtcatttacacatggtctgtttttctggaggacaattttatttgataaacaattgtttctatctgaatagaataaacaaggctctatgatgaagtaaaacactaaatacacatgcattaaaaaatgcataattatctttttggaatgggctatacagagatgtgctttttaaaatgttaagagtgtaaaaggacaaacagtgaaaaataaatcttcctcttattttgtcctccagtctcccaattcctctactcagaggtgagaacagaacttccacaccctccagaacctccacagttagaactgtctacatgtttccattgtctttacttttattcttgcctgcacaaataaatgaattgctcattatggaaacttcccaaaagacccgttaacacttcaataggaagcaccaacagtttatgccctaggactttgttcccacaatcctgtaacatcatatcacgacacctaacccaatccttatcaagccctgtcaaaaacggactttaaaccaagctgcaaattttcagtaatctggccttgcctttccccctctgatagcaccatcaaacaaacccccttactgccgaaagcaataagcccggctttgttccatccactggttgtgttggtgatatctggggactgccactgaacagacgcacagagggagcccctacaggcagggggtttttctgtctgtgcttctgggagagtatgtctcgtacatttgtcgcgttgatgaagacttcacagctccatcagctgcgggcaagggggtctgaggcagtcttaggcaagttggggcccagcggggagaagttgcagaagaactgattagaggaccccaggaggcttcagagctgggcgaggtagagagtctcctgtgcgccttctctcctctctgcaattcggggactccttgcactggggcaggcccccggccaggtgcatgggaggaagcacggagaatttacaagcctctcgattcctcagtccagacgctgttgggtcccctccgctggagatcgcgcttcccccaaatctttgtgagcgttgcggaagcacgcggggtccgggtcgctgagcgctgcaagacaggggagggagccgggcgggagagggaggggcggcgccggggcgggccctgatatagagcaggcgccgcgggtcgcagcacagtgcggagaccgcagccccggagcccgggccagggtccacctgtccccgcagcgccggctcgcgccctcctgccgcagccaccggtgagtgccgcggtcctgagatccccgggccggatgcgcggcggccccagctcccgagcgtctgcctcccccgccctgggctgcccgggctccctgggctccccggcggctgcacggagtcaaggcgccccgtcccgggcgtcccccgcgggtgccgatccaggctgcccggagtccggagcccagagaggagagagacagctggggagcctggtcaccgcgggcatctcccctgcgctgcagtcgcccgcctggcctgccttcccgttcctccgcctcttgccctgacttctccttcctttgcagagccgccgtctagcgccccgacctcgccaccatgagagccctgctggcgcgcctgcttctctgcgtcctggtcgtgagcgactccaaagtgagtgcgctcttgctttgactgatgctgcccaaggacctctgatcagcaccaggggagaggaggggctgctcagggagctggggtcctccggattccatccacagcagggccagactctccccaggaaatgggacagggtggcagcggaggcttgagaaccacgggggttggcactggctggcaagggaggaagaggccgccgggactgccccagcctgcgggcatctggtagatgaagcttgcttgggtcaatccatttctcctggctggaaacccatggtcttccatttgagaactagatacgaacagggtgaggcgagagggagagggaagagtgggttttgggattggggccagtttaccctcaccctggagtccctggagcatgggacctttgatgaagcctcctcccgaatctcttccagggcagcaatgaacttcatcaagttccatgtgagtatccacccctacaacagttggctgcacagacaagttgggaaggcttcaggggacatcccctccctgccctctgctgcagggctgcgccaccccttaccacttccactccccctcgcttaccccacctttgttctctccagcgaactgtgactgtctaaatggaggaacatgtgtgtccaacaagtacttctccaacattcactggtgcaactgcccaaagaaattcggagggcagcactgtgaaataggtatggggatctccactgcaactgggagagaaatttggggacagggagggatgggtgggaggcaagagcaggcaggagttaggagctggaggtagggtgggtgacatcttcatccctatgtgacaagcataaacacacacacacgctcacgaaacagtggccacacaaatgtgaggtggggttggaaggagaccctgtccagtcttctggcaggtctgaaacgacatctttaaaatgtccgttggcagccgggcatggtggctcacgcttgtaatcccagcattttgagaggtcaaggttgagtggatcatttgaggtcaggagttcaagaccagcctggacaacatggtgtaaccctgcctctactaaaaatgcaaaaatcagcctggcatggtggtggatgcctgtagtcccagctacttgggaggctgaggcaggagaattgcttgaacctgggaggcagagatctcagtgagctgagatcacaccactgcactccaactgggcgacagagcaagactccatctcaaaaaaaaaaaataaaagttagttggaatgttcttctctttctcatattctctcatcctcctgtccccttgtagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggtgaggtgggggcaacaaggaccaaaagccctccctacagcttcccagaaaccttgttaccatccccttctcccagagggctggccatagcacaagagaagtgcggcctctggttgagtcttccctgaggggaggaggcagggaaggccctctgggttggaatgacatcccctatctttctgtgttgccaggaacccagacaaccggaggcgaccctggtgctatgtgcaggtgggcctaaagccgcttgtccaagagtgcatggtgcatgactgcgcagatggtgagcatcactgacctgctgatgacagtggggtggaaggggacaaacttacatgtccccttattccatcacaggaggactgaggaggtggggggtgcccgagagggatgctttctcctacctgcctccctaagacatccctctgtttgtcctccaggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcatgtacggccctgggtttctcctcttcgactcttctgccccaccccaagcacatccctttctccttcccagcaaagtgttccgcctcatttctccctcatctgcccctgtccatgcagcccatggccttggggacaagtcgtgctttgaggcctctagggagggaaggaagaagtggcagatttcatgggactaagctgtttgatgggtatcttcttccacagtgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattggtgagggggaaccccgcgactactgtggccataatggcttggggagagtgggacccagggagagactggagctgaggttgaagctgcccggtggggcaggggtggggcgagggaccttgaagcctcgatatacatgacaaagggagtggcagggaagagttccatgaagtctgaggggcctggtgctcctctggagagaccctgaatttccccaacaagtagcctcttgcgagtggaaacagccctgtgggtatatggcttgggctgggaaggccctgtttatatgaattagaaaaagacacaccttcctttgtgggatgcagcctctgtctgtgctaggatatagaacttggagaatggagccttgggatggattccagcctaactacctcagctgggagtttttgcagaaacgacctgtacagctgtatgcagtggctctggccatccaagcctttttcaacacctggaacaaagcccttggggcatggggcaggggaggtttccaggtgataagcgaccagcagacctccctggatgactgacctagggataggcatagctacttcctcggcacttggaggggacagatggggaccgcctaaccagtagtgatctttctcctctgaccctctgtcctcccccagccttgctgaagatccgttccaaggagggcaggtgtgcgcagccatcccggactatacagaccatctgcctgccctcgatgtataacgatccccagtttggcacaagctgtgagatcactggctttggaaaagagaattctagtaagtgacaattgcgactgacttagaaggtcctgaggagtgttttgacctgaaaatgagcccagcgtgatcaagggaagactgcagagttagaggtgggagcactgaggcggtggcagatgggtccagggatggatgaagagtgttgtttagggagcgatgggctgcaaaggtaaatagatggtaggggctataggtggagtaaaggctcagatttgcatggaagagaataagggccttccctggtagagatactttatggttcccctctctggcagactcccagtggacagataaatcttgatgcaaacgcctccctgttttctccacctagccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccaggtgagtgttccaagcatctctctccacctcttccatatctccccagagctcctgggcttgttccagccagcttaagggtgtctctctctagccaaagccctaagtagccagaatcaggagctcaggtctttgagggtttaaaccagtccttatgtgtttgccagacattaccaaaaaaatcccagctctgcgctagtcacttcagactgggggcacgagatcctagaaagaggaaacagtaaaagacaatgtaactcagtgcccagggtgtgttgtgaactataaatgatcaggtgttcaggagagggaggtgagtgccaacctgagggtcagggaggggaggctttaaaggaaatgtgacttgataggcatttgaagaggcagagggaagaaaggaaggtgtttcagttgaaagatacaaaactgagaaggaggctggcatattccgggtggggaggagaactagggtctgggagtgtggatggaatagtggcagatgacagggcttttaaagccaagcaggggattttaaacttgatgtggtagaaaatggggctgcgtcaggcacagtggctcatgcctgtaatcccagcactttgggaggccgaggtggatggatcacttgaggccaggagtttgagaccggcctggccaacatggtgaaaccctgtgtctactaaaaatgcaaaaaaaaattagccaggtgtggtggtgcctgcctgtaatcccagctaatcaggaggctgagacatgggaatcgcttgagcacaggaggcaagtttgcagtgagctgagatcacgtcattgcacgccagcctgggcgacagagcgagattctgtcctccccccgaaaaaaagaaagaaaatgggaagtcgctaaggactttgactgggaaactcttccctctctctggtatggttgggtgatgggatcagaaatcccctcctcacttctctagggctcatcttttgtatctttggcgtcacagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggtgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtattattctgcagcatgacctgtgaccagcactgtctcagtttcactttcacatagatgtccctttcttggccagttatcccttccttttagcctagttcatccaatcctcactgggtggggtgaggaccactcctgtacactgaatatttatatttcactatttttatttatatttttgtaattttaaataaaagtgatcaataaaatgtgatttttctgatgacaaatctccctggtgcttgtatgggaaggagttggagtacataaaaaggagaaaataacaaaggtggactgcaccctagagtttcttatgggactgcatctctggacttaatggagctttgggaggtagaggttaggagagctgtagggcagggccaccacagcacttaatgtatacaaagtttccccaaaccacaatcccatggctgtgtaagtagtgtacaggcttagtgtcagctcccaattgcctccttaacctgatgcctttgtgcagtgcacatcttgtacagctgtacatggtggtcttctttagggttaactccacaacagtctcttcactctgcccccaacaatccttgagctgacctcaaccaagaagaacacctgtggccaggtgtagtggctcatgcctgtaatcctaacattttgagaggctgaggtgggagaatcgcttgagcccaggagtttgagaccagcctgggaaacttagtgagaccctgtctctgcaaaaaataaaaaaattagccaggtgtggtagcacatgtctgtagtcctagctacttgggaggctgaggcaggaggatcataagccaaggagttcaagtttacagtcagctgattgtgccactgcactccagcctaggtgacagagcaagaccctgtctcaaaaaaaaaaaaagaagaagaagaagaagaagacctgtaagtgaaaattgctggccaggctcagtggctcacgcctgtaatccagcactttgggaagccgaggtgtgtggatcacctgaggtcaggagtttgagaccagcctgaccaacatagtgaaaccccatctctactaaaaatacaaaaaaattagccgggtgtgggggtgggtgcctgtaatcccagctactcgggaggctgaggcaggagaatcatttgaacccgggaggcagaggttgcagtgagccgagatcgcaccactgcactccagcctgggcgacagagggaagacttcgcctcaaataaataaaaaaataaagaaagaaagaaaattgcttatccagaatgccagcttgactctgtggcattcaggaaacaaaaaacataatttcccatcatctgtgtgtgggactgatgctggaattttctcagtgtgttaacagagcttgtgaccagccattgctatgcctatgattagggggcccagaatctcaaagctggatgatgtcctaatgggggtgactatatattttgaacaaaacatcaagttctgaaaagctggggtgtagaaggcaggttgggagataattgggtatcaaaaattagaattgccaagctgtgttaatgggttggggcagtgtttcttaatcgagtcatctgaggaccttttaaaatatgcacacctacactccacctgtagagattgtgattcaggtcagggctggctccaggtctcgtggttttagaaaagctccctcagtgttactaacatgcaccactgattgacagcctttggggacaattggtcagaatatatgaagtgcccagaaaaattcaagatatggtcagcatgagttctctgcatgaacagccagttttctgagtggggtttggtggcacaaggtctcctggatttcctggttaaaggctttctggaccgtgcttccctagagggcaagaactccaagtatttggctttttcttttttttgaggcagggtctcactctgtcacccaggctggagtgcagtggcgcaatcttagctcactgcagcttctgcctcccaggttcaagcgattctcctgcctcagcctcccaagtagctgggattacaagcatgcactaccactacccggctcatttttgtgtttttggtagagatggggtttcaccctgttggccaggctggtctcgaactcccgacctcaggtgatctgcctccctt

SEQ ID NO: 6 - DNA, 126462 bp, Homo sapiens
gatcacttgaggtcaggaccagcctggccaacatggtgaaaccccatttctactaaaaacagaaaaattagccgggcctggtggcacacgcctgtaatccccgctactcggggggctgaggctcaagaattgcttgaaccgggaggtgaagtttgcagcaaactgagatcgcgccattgcactctagcctgggcggcagagtgagactctatctcaagaaaaaacaaaaaacagaaaaacaaaaacaaacaaacaaaaaattggccagattaagtggggtgtggcttccccggaagccttcatgcgcaggctttgccagggctgacaaagttgtcccaggctggccacgtgggctttgatctctccccaactatttgctcccgtgtgatccctggagagaggaatggagacacttgaatatcagcaacagggggcaggatgcagatcctccctgctcaggaagacacgcgtgggtgtctccacccacgggagagcctgttgcaggcgccggagagagctcctggtgcctcctgtgggggctcctgcacttggccagggaagggcctcctctttggtgttttttttttttttacgtttatgtttatgtttatttttcgagatagggtcttgctctgttgcccaggctggagtgcagcggtgcgatctcagctcactgcaacctctgcctcccgggttcaagcgattatcctgcctcagcctccagactagctgggactacaggcccctgccaccatgcccggctaagctttgtatttttagtagattttagattttaccatgttgcccaggctggtctcaaactcctgacctcaggtgatccgcccacctcagtctcccaaagtgttgggattacaggcttgagccaccacacccggcctccttggtgtttagatggggaagggggtgtatctctgtgatctgtcagggtgttcccaggcagggcactaggccagctcctgcctcatcaaaggatcaagactggaatttccctccatttcctatcattttagtcctcatctgaggaccacagctgccaagtctgtctctcacgagcacatgctgggcataggtgaggggcagagccctacataagcacccctatgccctgtccaacctccctccagaagcccctgtcctcagacagaaggtgcatcctctccactttgggtccttctccccttccaagaaaggggttcagggcaaggagagcttaacgcagctccaccactcactaactgggtgacgtctgcagaggttacttagcctctctgaacctctgttcttcgtctgtaaaatgatcacagtaagatatgccacggggttcctggaataaatgagattaataacataaacaaaccaggtgcagtgacttgtgcctgtaattccagctactcaggaggtcgaggcaggaggatcacttgagtccaggagttcaagaccagcctgggcaacatagtgagaccctgtctctacaaaatacttttttcttttttgagacaggatctcgctctgtcagccaggctggagtgcagtggcgcaatctagggtcactgcaacctctgcctcctgggttcaagccattctcctgcctcagcctcctgagtagctgagattacaggtgcctgccaccatatccagctaatttttgtatttttagtagagacggggtttcaccatgttggccagattggtctcaaacttctgactttaagtgatccaccctcctcggcctctcaaagtgctgggattacaggcatgagccactgtgcctggcccatctctacaaaatatttaaaaattaacaaggcatggtggtgcgtgcctataatcccagctactcaggaggctgaggtgagaggaccgtttgagtctggaagtttgaggctatagtgagctatgatggcatccctgcactccagcctggatgacagagcaagactgtctcttaacaaaacaataggccaggcgtggtggcttatgcccgtaatcccaggactttgggaggccaaggtgggcggatctcctgaggtcaggagttcgagaccagcctggccaacatggcgaaaccccatctctactaaaaatacaaaaattagcctggcgtggtggcgctcacctgtaatcccagctattcaggaggttgagacaggagaatcacttgaacctgagaggtggaggttgcagtgagccgagcttgcgccactgcactccagcctgggtgacagagcaagtctctgtctcaaataataaataagtaaataaataaataaataaatataacaaggccagggcggggtgttggatgatcctaatcactgctgttgtctagcaatgacaatcagagacatcctcaccacttggagacacatctgtcccatggcctggacagtgacattttcgggacaggacaccaactcttctgaacaattggagaccccatgccaagcatgatctctctggattcccccctttctgaagcctgcagaacaccctcctaacgcccgcagaacgccctcctcgaactggcattttccttcagacggacagtgatgctgttgcttctctaagttgcaatcacaattgcgtcagctcctctgaggttcctcccaggtctccttaattggatttggaacatttgcagacagacagtcgcctaaggtaacttctagaaggctaatatcctctcctaaactgagtttcttcctaattaagttctcagaaggaaatgttcactttggagtacttcactgttgggcccaagcactgcccattgtgcagtattcagtttatctgttgttggctttttttgtttttgtttttgtttgtttgtttgtttaagacggggtcttgctctgtcgcccaggctgaagtgcagtggtgcgatctcagctcactgcagcctccgcctcccaggttcaagcaattctcccacctcagcctcctaagtagctgggattacaggtacctgccaccatgcccggctaatttttgtatttgtagtagagacagggtttcaccatgttggccaggctggtctcaaactcctgacctcaggtgatccacacacctcggcctcccaaagtgctgggattacagatgtgagtcaccacgcccggcccgtttttgttttttcagagatagggtctcactctgtcgcccagcctggagtgcagtggtgtgatcatagctcactgcagcctccaactcttaggctcaagcaatcctcccacctcagccttccgagtagctgggaccacaggtgtgcgccaccatgcccagctaatcattttattttttgtagaggagggatctcattgtgttacccaggctggtcttgaactcctgggctcaagtgattctcctgtctcagcctcccaaagtgctgggattacaagcatgagccactgcacttggcccatatcatgcagtattaaggggcataggaggaaagtgtagacccaaacagttcctaacccctaaattcttaccatccagtagggaaagcaaaatgaatacatggcagtattttcagtagagacagggtttcaccatgttggccaggctggtctcaaacccctgacaatggaaagcaagccagtgctgaactctgtgtgtgatcctgtgggtcctattgcaagaattttcaggttcaaaggacagacagcagagggtggtttggggggaaaggtggaagccctagaaggctctgtggaccacagatctgcaaaggaggggaaacattccagagaggagtgtgtgtggccggagactggccacaaaagacccccagaagttgaagtgggggcagcgagtggacctgcctaattgcaagagatggatggcaaaaacaaaaatcagatcttgtgatgggaggagaggaacagatgtggcaaatctagatgtgcccccaaccccgtgtttacagataacttcccagtttcccctttcacccacacagcttctgctcatcttctgcagaacaggcacggcggggtgggggccaggcgcagtggctgacacctataatcctagcactctgggaggccgaggcaggtggatcacttgaggtcaggaattcgagaccagcctggccaacttggtgaaacctgtctctactaaaaatacaaaaattagccaggcgtggtggcgcatgcctgtagtcccagctacttgggaggctgaggcaggagaatcgcttgaacccgggaggcggagattgcagtgagcagaaatcgcatcgctgcactccagcctgggcgacagggcaagactctgtctcaaaaaaaaaaaaaaaaaaaaaaagaggcactgggtttgttgggaaacgtggggagccctggggaggccctgcaattaccacagccctggcagacgcccattacaaaagcacaaacacacacacacacacgcacgcacgctcattcatccagccagcggaactaacggtctactggacaggaaagccctgttggagagagagggtggggaatcccgcccaggccaagggggacagccctgctgccttccacctgctgcagagctgctgaccggctctgattcaacccggaaaaccagcccggtgttttggacacaacatgaccttttacctgtgggcctctcgggattctcagaaacagcaacaggtggggggttggggggttcccgagcctggggctttccttctgggctcagcccccacctccgcccccacttccctgatgagctgctaccaaagatagacaagagcaaatggagtttggcatgtcatttcctgaaaggatggggacttgaagacaaggtcccctgggcccagttcccggagtttaggttcaagaacgagatgcttgatccacccaaagcagaacggcttctgctagctctaaatcggtggctcagaaagagaagggggctcccaaacctcctagaccctaaagctcctctgttggtctctctaatttccctggtgtcccctgtccaggagcaaggccgtggacacacaacaactgcctctgcttcctgagatgctgatccttggaccaagctggccagtccagggctgaagggctcaggaggagggagaggtctctgcctgcagggccccaagcatctgctgtccctggcacgacccgggttgtctgggtctggggccagtctcctaggggaatgagctggcaggctgggttgtacccagggagtgtgcctggagggagacagccgtcgtggttcaggctgtgaataacaggagcctcatctctgccccagagcttcagggaagggagcacctggcacagaagtgactaagagagcaggtctgggcgggtagctgacgtgcagcaaacaagggaataggaggttcccccaggttcccacacacggagcaaatcccacggcctgccctttggggtcctccatgtccagaccctccaacctggtttctgcacacaactgcttctccaggctgagaagtcctcccgaccagcccccaaccatacccctctgctctatgccaaggtcactggacccttccttgtctgctccaggcctcacacatcccctctctaggggcctaatctgatctcatgtgtcactcaagagcctcttggtgtaaatctccttagaacctttttttttgaaacagggtctcacgaccaggcacagtggctcacgcttgtaatcccaacactttgggaggccaaggtgggtggatcacaaggtcaggagttcgagactagcctggacagcttagtgaaaccccgtctctactaaaaatacaaaattaactgggcatggtggcaggtgcctgtaatcccagctactcaggaggctgaggcaggaaaatcacttgaacccaggaggtggaggctacagtgagccgagatgatgccactgcactccagcctgggctacagagtgagagtctgtctctttaaaaaaagttgtgaaggaaggggccaatgaaggaactcatagggagcagtgggtcacacggtgaggggcttgaagtatcctgggttggggataggggacaggcatgcaagggtcacgtcgggggaacagctgagatacggagaaacaggatgaagtctggttaagagctggggctgctggagccagtctctctcttagctgagtgactttgagcaggatagcaaatcctctatctgtcagtttcctcatctttcagatgagaataggaccaataccttattttgttattttaagagaaagagtctctctctgtcatccaggctggagtgcagtggcatgatcatagctcactgcagccttgaactcctgtccttaagctaccctccctcctcagcctccggagtagctgggactacaggtgcatgccaccacacccagctaattattagtattattacatttttttctttggcggacagagtcttgctatgtttgcaagacagagtcttgctgtgttgcccaggctggtctccaatccctggcctccagttcctggcctcaagtgatactccagtctcagcctcccaaagtactgggattacaggagtgagcccccacatctggtccccaatacccatttttaattaattatttatttattttattttttattttttcttgagatgggagtctccctctgtcacccaggttggagtacagtggcgtaatctcggctcactgcaacctccacctcccgggtttaagcatttctcctgcctgagtctccctagtagctgggattacaggcacccacgaccatgcccggctaatttttatatttttagtagagacggggtttcaccatgttggccaggctgggcttgaactcctgaccttaagcaatctgcctgcctcagcctcccaaagtgctgggattacaggtgtgagccaccgtgcctggctgtcaataaccatttcttagttgtatgaaaatgaaatagaataatgagtatgaaacagggcctagcaaacagcaactgctatatgtatcttagccctgttattgctgcagtttggggatgggaacttcaaaaactctaaagaaaaggcaagcagggccgggtgtgatgggtcacgcctgtaatcccagcactttgggaggccgaggctggcagatcacctgaggtcgagagttcgagaccagcctgaccaacatggtgaaaccccatctctactaaaaatacaaaaattagctgggcgtggtatgggaggctgaggcaagagaattgcttgaacccgggaggcagaggttgcagtgagccgagatcgcgccattgcactccagcctgggcaacaagagcgaaactccatctcaaaaaaaaaaaaagaaaggcaagtagaccctgagagctgaaaaacaaatggattcgctgatcaaagccacctaaacaaattacagaagcaaccacaaatccagactaaggcgggcaggaatttttagactctttgcctcaatcttccctccagccctgccagcatgggagactggcctggggaatcccgggggcactgtgtcgtttcttctgtcccttccagatggaagcttcttttctagccctgggagaaggctcattcctgctctgctcaaggctagcctggtctgggccctgaaagaaaaggaaattgtcccctgtagtaccacagagggtggccctagactgacctctttcacccctaatggagctttccacaagctctggctcctgaaaatcctgccctgaaacctggagcccactgggcatggtggctcacgcttgtaatcccagcactttgggaggccgaaacaggaggattgcttaagcccaggagtttgaggccagcttgagcaacacagcaagaccccatttctacaaaaatttttaaaaattttttgagtgtgctgggtgtgctggcgtgcgcctatggtcccagctactagggaggctgaggtgggaggatcacttgagcccgagagcttgaggctgcagtgagccatgatcatacctctgaactccaacctgggtgacagagagagaccctgtctcaaaacaaaatcgaaaacaaatgaacaaaaaaaaacaagggctgtcttatttggtggccagcagctgactggccattgagggctatggccattatggcctcagccacagtggaacctccttagctggatcctcctccagttcttcccttgatcccctgactttggcagtggaatcctgttcctccccataggagcatatcagccttgaaaggccagaaaacagtgcagggacccatttcttccagctggtccaacgcggtgccccacccagcactccagccctgcctggaagacgctagatgctgtgagcttccccaggcctgggttgtttaccccacttggggcaggaaggccctgtcccttcccttgcccccgtggggatgactctatcccctacgcattgtttcccttccgctggaatttctgttcttgaggacttcatgttctacccctcgcataacggtttcctcctgtccagacagggctggccaccagccaggaccttgaccattcgtgcagaggatgggggccgaacacagcaaagggttgaagcaggaagagggaagctggaattcctccttggtcagggccttggggccccttctgcaggggcctgcggtgcatccactgtggatgggacttcaggcccaggccaccacgccagaatacatatgtatatatatttgtattttctgtggagacggtgtctcactattttgcccaggtcagtctcgaactcccaggctcaagggatcctcctgccttggcctcccaaagtgctgggattacaagcatgagccaatgcacctggtccccaatacccatttttttagtttagttgtatgaaaatgaaacaaaataatgctatgaaacagggcctagcaaacaaacagcaagtgctctgtgtatactagccctgttattgcagaagttaggggaagggaacttctaaaactctaaagaaagccgggcgcggtggctcacgggtggaatcccagcactttgggaggccaatgcaggcagatcagttgaggccaggagtttgagaccaccctggccaatatggcgaaatcctgtctctactaaagataccgaaaacagccgggcgtggtggcgcatgcctgtaactccagctactcgggaggctgaggcaggagaatcgcttaaacctgggagacggaggttgccgtgagctgaggttacaccactgcactccatagcaagactttgtcaaaagagaaagagagagagagagaaagagcgaaagcgagagagaaagaaagaaagaaagaaagaaagagagagagagagagagaaagaaagaaaagaagagaaagaaagagagagagagggaaagaaagaaagagagaaagagggaaagaaagaaagaaaagaaagaaaggaagaaagaaagaactgtaaagaaaaagcaagccagctggacgcagtggctcatgcctgtaatcctagcaccttgggaggccgaggcaggtggatcacctgaggtcaggagttcaagagcagcctgaccaatatggtgacaccccatctctactagaaataaaaaaattagctggacgtggtggcacgtgcctgtagttccagctacttgggaagctgagacagaagaattgcttgaacctgggagatggagcttgtagtgagcccagattgtgccactgcattccagcctgggcaacagagggagactccatctcaaaaataaaataataataataataataaaataaaaagtaagctggccgggcacagtgtctcatgcctgtaatcccagcacttttgaaggccgaggcaggtggatcacttgaggtcaagagttcgagaccagcctgaccaacaaggtaaaaccccgtcaactccacctctactaaaaatacaaaattagccgagcgttatggtgcacgcctgtaatcccagctacttaggaggctgaggcaggaggatcacttgaacccgggaggcggagtttgtggtgagttgagattgtgtcattgcactccagcctgggcgacagagggaaactccgtctcaaaaaaataaacaaaaagaagaagaaaaagcaagtggtccttcccctctgcagtggagcagtgtgagtccatggagcaaacccagactggacagatgaggaagttccacgcaggtggggacacaccttaccaccttgaatttcatactcccaaatcgggttctcccaccatttgctgcaagactctcccagctaatgaggaaggcaagcagctcagatggacaggtaacagttgaggctaacctccaatgtcatcacagggcgggggtggttctgcccaatacctgtgccaactctcccgcccatggcactccctgtagggttttgtgaggtggggagaagggggagatgcggtggggatggcgatggcctcctctcccctgtcttagcccctagcctgagtgctgattgcaggaaagagcccaagtctgatcccgggggtgctcaatgcgcccagatcatcctgccctcccagacagagcagaaggggccccggagccctctgtcaagtcttagctgcctcatatctgccacctcagcccccctcaccccaacagccccggctccctcccccaggcacccgggaaggcagtgctgttgtgtgaataacagttacttgtcctgcctgcatcagccgttactcacacagtcccgggagatcctgacacagatgggggtgggggcacagaggggactcatcagagccccccaggaaggggactcagggggagagcatgtcataactctcagatggggcttcgggccctgggagtagtgggggcatcaggattgcagcctggaggggtcagctttggatccgagttctttgtttgcttcttgtctttttaatttgtttttattttttaatttttatttatttatttattttgagaccgctctgtcacctaggctggagtacggtggcgcgatcttggctcactgccacctctgcctcctgggttcaagcaattcccaagcctcagcctcccaagtagctaggattacaggcgtgtgccaccacacccagctaattttaggatttttagtagagacggggtttcaccatgttggtcaggctggtcttgaactcctggcctcaggagatccacctgccttggtctcccaaagtgctgggattacaggcatgagccaccacacctggccttttttgtcttttttagagacagggtcttgctctgcagcctaggctggactagagtgcagtggtgtgatcacggctcactgcaaccttgacttcctggtctcaagagatcctcccgcctcagcctccccagtagctgggaccacaggtgtgcaccactgcacctagctaattttttaatacatatattgtagcgacagggtctggctatattgcccaggctgatctcaaactccttgcctctaatgatcctccagtcttagcctcctgaagtgctgggattacaggcatcagccacctcacccagcctagatccaagttcttactgtggtattaatgacctagttgggcctccttttccacagttcttacaggccagtgccagacacaggccagagcccttggtggccttccctgccccagcaggccctggcccagcacgacctgctgactgtctggagtttcatcaccttagagcctcattggtgaatccccttctagcacaggagaccctcaggaaaccagtgggctcaagaaaagatatattttttaaaataattacttttctttttttttcttttggagacagaattccaccctgtcacccaggctggagtgcagttggggcgatctcacctcactgcaacctccacctcccaggctcaagcaattctcctgtctcagcctcccgagtaactgggactacaggcgcgcaccaccacacctggctaatttttgtatttttagtagagacggggttttgccacgttggctaggctggtctcgaactcctgacctcatgtagtccgcccatgtgagccgtacgccaccagccacgatgcccagccaaataattacttttttttttttttttaatgagatagagtcttgctctatcaccaggctggagtgcagtagtgcgatctcagctcactgcaagctctgcctcccaggttcaagcgattcccctgcctcagcctcccaagtatctgggactacaggcggccgccaccatgcctggctaatttttttttttttttttgtattctagtagagatggggtttcaccatgttggccaggatggtctccgtctcctgacctcatgatctgcccgccttggcctctgaaaatgctgggactacaggcaagagccactgcccccggccaccatttttgcaacctactaacgactttctatggatggatgcagttggtcatttcattcataccatccgctgaaatcctaatgccactgttatggaattggcatcattatctctactttacaggaaagaaaattaagactcaggggagaaatgggagttgcccagaatttcctgcttgtacatgtgccctgtcggcttctttctccagagactggggctctagaaggtaaaggacctgttcacgatcgcccagctcactggagccagagctgtctggttatagcaatggccatgggagaagtctgcgtccagagcaaacacaaagggctcagtagcaggttggatttgaatcctttttttttttctttttttttggagatggagtctcgctctgtccaagcaggaagaggccaggcgaggtggctcacccctgtaatcccagcactttgtgaaactaaggcgggcagatcacttgagatcaggagttcgagaccagcctggccaacatggcaaaaccccatctctactaaaaatataaaaattagccaggcgtgttagcatgggcctgtaatcccagctactcgagaggctgaggcaggaaaatcgcttgaacccgggaggggattgcgccattgcactccaacctgggtgatagagcaagactgtctcaaaaaaaaggaggaggaagccaagtgtgggcagcctcgggaaagtcctcctacgtggcattcggtctgcactggagtagagggcgttgctcaggacagagactacctcacaagctcctgagacaaggacagtgttggtcctatgcatctgcgaaggtcccataacaaccaccccagaaaagaccctaagatgccactccatgctctcactggctgtgttggaggaaagcaggaggcagccatcccatgtcccacacccccggccaggtcaggctccctgctcccctcctctcctacacctcactgctctccagccctccagccacaaacctacacaagggggtctgggatgggctctgggcccagacagatccaagcaagccagcagcccagctgcatgacagtggtgaggagagctgagggggcccagggtgactcagatgaagagacatgcaagaagcctgctcccatatggaggggaagctgggcagataagaatggccagtgactctgggcatgctggcatcccatggtgaattccatctgcactgtatcacccagacggcttcatccctgggacctccacccccacccatcccccactgaccttgggtcaccgtctctctggacacctgcccagctcagtgggagctcagtctttatagtcacatccagagccagaatggagactagcaaaagaacgtctgtgtccaagccaggcgtggtgttgcgagcctatagttccagcgactcaggaggcagaggtgagaggatcacttgagcccaggagttcaagaccagcctgggcaacatagtgagacccaatttctttgttttctttcttttttttttttgagatggagtcttgctatattgcccaggctggagtgcagtagcatgatctcggctcactacaacctccacctcccaggttcaagcaattctcctgcctcagccctcctgagtagctgggactacaggtgtgcatcaccacacccggctaatttttgtatttttagtagagacggggtttcatcatattggccaggctggtcttgaactcctgacctcgtgatccgcccgcctcagcctcccgaagtgctgggattacaggcttgagccactgttcccagccgagtgagaccccatttctacaaaaatgttttaaattagccaggcctggtggtgtgcgcctgcagtcccagctactcaggaggctgaggcaggaggatggcttgagcccaggaggtagtggctgcagcgagccatgatcgtgccactgcactccagcctgggcaacagagcaagaccctttctcaaaacaaacaggaacaacaacaaagaacagaatgtgtccagagaaaggcgtaaagagagacacaagaatgtagagaatgaggaacgagttctggtaaagatgaacgaaagtgaatcagagtcagataagccacatatgtgggtaaagagaagtggatggagggatggaatctggccagtggcctggaggcaccctgcaggcctgcactacctcatcagcctgacgtaatcagctccatgttcacatctaccccaggagcccggtgctccaccattccctgccattgaaattccccttggtgaccacactgggggcacattccttttctcttttttttttttttttttttgagacagagtctcactctgttgcccaggctggagtgcagcaatgcaattacggctcactgcagcctcaaactcctggactcaagtgactctcctgtcttagcctcccaagaagctgggactacaggcacatgccaccatgcttgactcatttatttattttttgtagaggttgggggggggggtctccctgtgttgcccaggctggtctcgaactcctggcctcaagcaatcctgccaccttggcttcctaaagtgttgggattacaggcatgagccactgtgcccggccaggaaggctcattcttgcctgatggtgaaaggacatttggaatacccacattacaaaagacccagcactaagtgccctcaggaaataagacactttgatggccacataagtttaaatacagaaataactgtaagacaagggagaatgcaaatatataaaagtgataaaagctaaaaattagctgggtgtggtcgtgcatgcctgtggtcccagctgctcaggaggctgaggtgagaggattgtttgagcccaggagttggaggctgcagtgagccatgatcacacaactgcactccagcctgggcaacaaagtgagaccctatcttaaaaaaaaaaaaaaaaaatagactgggcgcggtggctcatgcctgtaaacccagcacttcgggaagctgaggtgggaagatcagttgagctcaggagtttgagaccagcctggccaacatggtgaaaccctgtctctactaaaaatccaaaaattagttgggcatgtttgtgcaggcctgtagtccctgctactcaggaggctgaggcacgagaattgcttgaacccgtgaggtggaggttgcagtgagctgagatcgcgtcactgcattctagcttgggtgactgagtgagactctgtctcaaaaaaaaaaaaagataaaattggctatggtagagcatacattttttttaagtgattaaagcattataggtgccatagaggaatcgatgagagatgggagatggaggctgcaggggcctagcctggaagagtgggtcgagctcaactccattttggtaaatcaagtctccttcctccctggtatgctcagaaaataaggatacacttgggacaaaagcggggtacatccatgacctgcagcccagaggggaatatcttcctgggatctgaaacctgcaaagtcagccaatgggagcgtgtcaggaccagggagacagcagaaggatctggtatgaaacagatgagaggggctgccaggcatcccctcgggtaagcagccagcccacttccgaggcatttaacgcccacgctccgctaaatgctgagagacgtaaaaacaaacacctcaaacatccatccctcaaggagcttgcattatatcaaaatgaacagctcagactgtctgggttcctgtcctggctctgctgcccaccaagagcggctgccacaaaggagaacatttgttcttgccaaatgtgttggacatttggcagatgacgttttatgtaattctcattgcaaccttgaaagatgggaatcgttatctgtcttttacaaataagaatactgaggctgtgatccctaaatcatagggaaggaaggaagggagggagggggaggggaggaagaggaggaggaagagaaggagaaggagaagggattaagttttacctagtcacatagccaatgtcagattcctaactagtggccgggtctgtctgatccaatgatcactattctctcatttatggtgtagtcactgtgtggcttcaaccacagtggacctctctggacctaagtgccctcacttgtaaattaaaagaactgggttagggccaggcatggtggctcatgcctgtaaccacagcactttgggaggctgaggcaggtcggtcacttgagctcaggagttcaagaacagcctgggcaacgtggcaaaaccccgtctctaccaaaaatacaaaaaattagccaggtgtcatggtgtacatctgtggtcccagctactgggaggctgaggtgggaggatcacttaatcccgggaggtagaggctgcagtaagctgaaatggcaccactgcactccaaccttgagcccaggagtttgagactagcctgggcaacataaagagaccctcatctctgaaaaaaaaaaaattaggcggggcacagtggctcacatctgtaatcccagcactttgagaggctgaggaaggcggatcacttgaggtcaggagttcaagaccagcctggccaacatggtgaaaccccgtctctactaaaaatacaaaaatcagctgggcgtgatggcagatgcctgtaatcccagctacttgggaggctgaagcactataattgcttgagcctgggaggtagaggttgcagtgaactgagaccacaccattgcactccagcctgggcaagaagagcgaaactttgtctcaaaaaaaaaaaaaaaaaaaaaaaaaaattggcggggcatggtggtgcacacctgtagtcccaactagcaactcctcaggaggctgagatgggaggatctcttgagcctgggaggtcgaaatgcagtgagtcatgactgtgccaaggctgggagccatgattgtgccactgcactcccacctgggcaacagaacgtggcccccactcaaaaaaaaaatgcaattgtggacaagcaagggctacattgggtaaggcagcaccagttagagtgttagggagtttaaagagaaatgaccggccaggggctggcaggggagaggggatacagcaggagaggagggggaagtcagtcacagagccagggtaaggaagggcccctggacaggctgtcccagagccacgatcttaaataaaaggcagtggagggccaggcgcagtggctcacgcctataatcccagcactttggaaggccgaggtgggtggatcatgaggtcaggagatcgagaccagcctggacaacatggggaaactagtctctactaaaaatacaaaaattagccaggcatggtggcgcatgactataatcccagctactcgggaggctggggcaggagaatcgcttgaacctgggaggcggaggatgcagtgagccaagatcacacctccgcactccagcctgggcaacagagcaagactccatctcaaaaaaaaaaaaaaaaaaggcggtggagacacccagcactggttccctcctagagaaggggagagagccgagcccaaacacaggctatttttggagttgtgttgtttggagagcaaccaagccagaaatagtgctgttagcaagaccctgccttctccaacccccggctctcccagtgaaatcaccaggaatcagggaggctgggccccacattccagggagcctgtgtaacaggcagtgctctcttccccaccggaagggcttgggagctgagcatttgactgtaaaagggattctgggattccagagggacagagggactgagtcagagagagagagagagagagagtggaaatggggggcaacctcctgcagcggaggagaggggacaggagggaaggctcggggaagaaagggtaggcaggaggtcagagagaagggagcaggaagcgggaggctcagagagagaaagggaaagtgaaaaagaaagcggggaagacagaagcaaagaggaagagcagagatcgggccagaaggcgggaggtcagggggcgatgggtggtgggaaggccagtgtccacccagggcccaccagtgggtgggggacagagggacagtcatggaaaatctgagtgtgatctacagtcacgaaccacctgctcagggacagatggagacatctttcccagtagctggaatcagaactttcccctgaaatccacagcccggtaactcactctcttatctctgaccctccggaagccagagatctagctatttcatcagcatggacttttctttctgtgcccgtgtgaccccttacaaaagagctcaagctggaaagagaggattggatcaagaaagagagaacttaggattcaggggaaaaggggaaagtgataaccccttgagtcagattctgagaagagggtttctctgagaggtcggtggggagcagcccgcaggcagctggaccctggtatggaagggctcggctcctaaggcaggtaagagaaggcagggccacgtgcatgaagtagaggcaggggtcatggccaagtctgggttctttttatttatttatttttttgagacaatcttgctctttcgctcaagctggagtgcagtggtgcaatctcagctctctgcaacctccacctcttgggttcaagagattctcccacctcaggctcctgagtagctgggactacaggtacgtgccgccatgcctggctaatttctgtacttttttttttttagtagagacggggtttcactccaagtctgggttctagagtagctaggcagccccagctcatcagagtcaatggagaaatcggaaacttcacatggacccgataacacaggttttgcctcagtttctaatacacatcttccctatgcctacaagtttccaccgatttgggaaaagacctcttagtaagtcagccacttctgagtccaaagtaaaacgctccccaccctccaccccaatcaggactagagctgctccccagctctcccgtgcccaaggatggaatgtcctttttgctctttgctttgtcctttgagacagggtctctctttgtcacccaggctggagtgcagtgttgcaatcatagctcactccagccttgagctcctaggctcaagcgatcctcctacctcagcttcccatgtagctgggaccataggcacacgccaccacactgggctaattttttttttttttttggtagagatgggatctcactgtgttgcccaggctggctcaaattcctgggctcaagtgatcttcctgcctcagcctccccaaagactgggattacaggtgtgagccaccacacccagcaggggtgtctattttggattggcatcaggatcatccacccaatctatgagtggggactaaattggtgactgctcctgcagagctctggctgtccacagaaaacagcagagacctggcaaggtatgggacaatggctcctggtatgacaggcagaggaagggatggaggctaagctgcagcctggagaggctggcgtcagacacctgaggaggccacatctagctatgtctagacccattgaactttcaagtcttcgaggcttggtactgcctgatccctgttcccctctgatccttaaggtcagagagcaaagccttgcggggtactttttttttttttttttttgagacagattcttgctctgtcacccaggctggagtgcaatagcatgatctcggctcactgcaacctccacctcccaggttcaagcgattctcctgcctcagcctcccaagcagctaggattacaggcacgcaccaccatgcctagctgatttttatatttttagtagagatggggtttcatcatattggccgggctggtcttgaactcctggcctcaagtgatccacctgccttggcctcccgaagtgctaggattacaggtgtgagccaccatgccctgtcttggggtacttttgaggacaagagttaagttcgggactgggcatgttggcttatacactttgggaggcaaaggtgagaggattgttccagcccaggaatttgagaccagcctggtcaacatagcaagactctgtctctataagaaaaaataataattagctgggcgtggtggtgcttgcctgtagtccccgatacttgcttgagcccaggagttcaagtctgcagtgagctatgatcacatgactgcactccagccttggtgacagagtgagaccctgtctccaaaaaaaaaaaaaaaaagagatgggtttggggctggacttgtcttcttccaagaggtcagggcagttgcttcttcctcaccgctaaccacactctcagggactccaaaccacaactaagaaaaggaaaaagacataaacaagaggcagtttcacaggaagagttctcctaatgtgtctgtgagcctaactttccgactgtggttctcctctctttccctgccagctgtgagaaaacaaagcctaaggcagattaaaaagcaggagggctgctcagtgtggttgcccttggtatctgtttactgaaaaaatccaaaacaaacatccgacgcttccacccactgaaacttcctgtgactctgctatcagctgttgccatggattctcccaactgaaccccaccacatacactaaacccaacagtccgtggaccccaagtatgccatctctccaatacaggaccccctgggcagcaccctggccacccttccatccacaacatccagaccacacggccaagggcacctgaccctgtcaaaaccccaaatccagctgggcgcggtggctcatgcctgtaatcccagcatttgggaggccgaggcagccggatcacgaagtcaggagttcgagaccagcctgaccaacatggtgaaaccccgtctctactaaaatacaaaaattagccgggcgtggtggtgcacacctgtaatcccagctactcgggaggctgaggcaggagaaccacttgaacctgggaggcggagattgcagtgagccaagatagtgctactgcactcagcctgggcaacaaaataagactccgtctcaaaacagaaaaacaaaaaaaacaaaaaacaacaacaacaacaaaatccactagtctagccctacatgctcacccactgctagccagtttccaccctctacagcagaggtacccaacctctgggccaggggcccgtactggtccatagcctgttaggaaccaggctgcataacaggaggtgagtggcaggtgagtgaaatttcatctgtagttacagccactcctcatcactcgcattaccaccagagctccactccctgtcagatcagcggcggcattagattctcataggagctcgaaccctattctaaactgttcatgtgagggatctaggttgcaagctccctatgagaatctaatgcctgatgatctgtcacggtctcccatcacccctagatgggaccatctagttgcaggaaaacaagctcagggctcccactgattctacacgatggtgaattgtggaattatttcattatatatattacaatgtaataataatagaaataaagcacacaataaatgtaatgtgcttgaatcatcccgaaaccatcccaccctggtctgtgaaaaaattgtcttccatgaaaccagtccctggtgccaaaaacgttgaggaccactgctccacagaatctatcggtcactcttcctcccctcacccccttgccctaaaagcacaccctgcaaacctgccatgaattgacactctgtttctatcccttttccccttgtgtctgtgtctggaggaagaggataaaggacaagctgccccaagtcctagcgggcagctcgaagaagtgaaacttacacgttggtctcctgtttccttaccaagcttttaccatggtaacccctggtcccgttcagccaccaccaccccacccagcacacctccaacctcagccagacaaggttgttgacacaagagagccctcaggggcacagagagagtctggacacgtggggagtcagccgtgtatcatcggaggcggccgggcacatggcagggatgagggaaagaccaagagtcctctgttgggcccaagtcctagacagacaaaacctagacaatcacgtggctggctgcatgccctgtggctgttgggctgggcccaggaggagggaggggcgctctttcctggaggtggtccagagcaccgggtggacagccctgggggaaaacttccacgttttgatggaggttatctttgataactccacagtgacctggttcgccaaaggaaaagcaggcaacgtgagctgttttttttttctccaagctgaacactaggggtcctaggctttttgggtcacccggcatggcagacagtcaacctggcaggacatccgggagagacagacacaggcagagggcagaaaggtcaagggaggttctcaggccaaggctattggggtttgctcaattgttcctgaatgctcttacacacgtacacacacagagcagcacacacacacacacacacatgcctcagcaagtcccagagagggaggtgtcgagggggacccgctggctgttcagacggactcccagagccagtgagtgggtggggctggaacatgagttcatctatttcctgcccacatctggtataaaaggaggcagtggcccacagaggagcacagctgtgtttggctgcagggccaagagcgctgtcaagaagacccacacgcccccctccagcagctgaattcctgcagctcagcagccgccgccagagcaggacgaaccgccaatcgcaaggcacctctgagaacttcaggtaggagaaaagcaaactccctccaacctcttacttcgggcttaaggcagagaactcgcctccccagaatctcctccctccatgatcccccgctattcctctattttcttttcctcggacctgcagccttgggtcgaccctgccctaggggtgactgcaggagagcagggaggatggtcaggcgtcaccaacaaccccatcacccagtaacaagaaccttgactctctcagtccctctgcatcaagacacttacccatttcccacctcatgcctgctaacttgaatgaaacaatcgctgggaaagcattaagagaattaaggctgggcactgtggctcatgcctgtaatcccagcactttgtgaggctgaggcaggcagataacttgagcccaggagtttgagaccagcctgggcaacatggcaaaaccctgctctcccaaaaaaatacaaaaattagctgggcgtgctggtgtgcctgtattcccagctacttgggaggctgaggtgggaggattgcttcagctggggaggcggaggctgcagggagccaagactgagccattgcacccagcctgggtgacagagcaagaccctgtctctaaaaatgaatgaaaggaaggaagaaagagagagaaagagagagaggaaagaaggaaggaagtaaagaagaaagaaagaaagaaagaggaaagaggaagaaagaaagaaaagaaagaaaagaaaagaaagcaaatttaaagcttatgcaaatcaaagatgttgtgataattgataattgagtctgggctaaattccccctgggctgcaaaggcagagagtggtaatgacttctcacctgcttttcttctaaggcttttttacgggacacagagggaagggagatggactggattccaagattcccacagggcaagatgggcgaagactccctgccactgcccggggataagtcagtctgagtgagacggagtgggatgggcttagaacctgaacatgtcatggtctcttcctgcaccttgccctagtgttcacttaccacctgcttgcaggaaacaagaagagcagggcccacagctggccagctcccctcccctcccgcctgtcttccagaacgattccttcaccagccctctttccattgctctaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatggtgagccacgggacaccaggggaggtgggtggcatgcagaacagacctaccagaagccaaggaaaggctggctctggcttagccgagccaagccccatacagctgtgctgcaggggccaccccatcttcttcccactacactccaagtcactggacccttgaatctccaagggtgtctgaccagtagatttaccgcttattcaccaccgtgtgatcttaacctcgttaagtttgcccatctacaaaatgaggattatttgctgtcctaaagaattcatgagccgggcgcggtggctcaaacgcctgtaatcccagcactttgggaggccaaggcgggcggatcatgaggtcaggagatcaagaccatcctggctaacacagtgaaactccatctctactaaaaatacaaaaaaaattagccaggcgtggtggcaggcgcctgtagtcccagctactcgggaggctgaggcaggagaatggcatgaacccaggaggcagagcttgcagtgagctgagatcgtgccactgcactccagcctgggcgacagagagagactccgtctcaaaaaaaaaaaataaaagaattcatggaattacacttgtgaaatacttagcatagccatcactataggaaaaaaatctaaggccaggcacagtgcctcatgcctgtaatctcagcactttcggagtttgaggcaggaggatcacccaaggctaggagttcaaggccagcctgggcaatacggtgaaaccccgtctctaataaaaatataaaaattagtctgatgaggtggtgcacctgtaatcccagctactcaggaagctgagacacaagaatcactttaacccgggaggtggaggtggcagtgagctgagatcacaccattgcactccagcctgggtgacagagtgagacctgtcaaaaaaaagaaaagaaagagagagagagagagaagagagagaaagaaagaagaagaaagaaagaaagagagagagagaaagaaagaaagaaacaaagaaacaaagaaagaaaggaaaagaaaaaaaaaactaaggccaggcaaggtggcttatgactgtaatttcagcactttggaagattgaggcaggaggatcacttgaggccagaagttcgagacaagactgagcaacagggagacccctgcctctacaaaaaaatttacaaattagccagatgtggtgacacatacctgtagtcccaactactcaggaggctgaggtgggaggatggcttgagcccaggagctggaggctgcagtgagctatgattgtaccactgcacttcagcctgggcaacaaagggaagccctgtctgaaaaaaaaaaaaaaagaaaaagaagaagaaagaaaatatttagggttcatccaggaggcagaggttgcagtaagctgacatcgcgccattgcactccagcctgggagacaagagcaaaactccaactcaaaaaaaaaaaaaaaaaaaaaacaggaagaaaatatttagggttcataatttaagaacagagaaaaatattctagcccaaagaaagggttgggatctgagacttttgaagaaaggaaggagatacagaaaagagatttcatcctggaatgaaatctccctccagagagccctgggaaagcacggtagcccccatccatcagagtggagccccttgtgggggaagtgggctcggctgggaaccctcaattcagcataagcctcacatgtcctctcctctctgtcccggtgcagacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtgagcaggcagggaaaggaaacccatttcctgggcctcaagagaaagggaatttggaaataaatccacatatcccagttgggtgcagtagttcacacctgtaatcccagcccaacactttgggaggtctaggcgagaggaaggcttgaggcctggagtttgagaccagcctggccaacataacaagacctcatctcttcaaaaaatttaaaaaccagccgggcatggtggtgcacacctgtagtcccagctacttgggaggctgaggtgggaggatcacttgagtccagcagttcaaggctgcagtgagctatgtttgcaccaccacactccagcctgggtcacagaacaagacctcatctctaaaaaacaaacaaaaaccaaatccacatatcctaaaaaatgctccttttcagcattctcttctctatggacaaagggctggatgctttaagaaccaaatcttaggctgggcacggtggctcacgcctctaatcctagcactttgagaggccaaggcgggcagattgcctgagcacaggagttcgagaccagcctggccaacatggtgaaaccctgtctctgtcaaaaatacaaaaaattagccaggtgtgttggcgcatgcctataatcccagctgctcgggaggatgaggttcaaagaatcacttgaacccgggaggcagaggctgcagtgagctgagatcatgccactgcactccagcctgggtgacagagcaagactttgtctccaaaaaaaggaactagacgggttcatttaaacccctgactgcagccctttgacatacatccaattgaggactggggactccgggaaacatctaaaaggcttaaaaactttgtctaacttcagccgggcatggtggctcacacctgtaatcccagcactttgggaggctaaggcaggtggatcaaaaggtcaggagtttgagacgagcctgaccaacatggtgaaaccccgtctctactaaaaatacaaaaattagccaggcatggtggcaggcgcctgtaatcccagctattcgggaggctgaggcaggagaattgcttgaaccccggagacagaggttgcagcgagccgagatcgcgccactgcactccagcctggcaatagagtgagactccatctcaaaacaacaacaacaacaacaacaaaatcgtctaacttcctgatcttcctgatcattgattttcccataggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatagtaagtccaagagccccttccccacagcccacagcaactgcatctcattcctggggtctcccaaggaatacccaaaatgtcaccctctgagggaggaagaccacagggaatgctcccctttaagggaggagagaccctagaatatactccagctttgacaaagatttcccaagcaggagacatcaggataatgggaacagaagacaggaggtttatcccatgaaggatgaagaagctgaaatccagagattccctcagggccacatttgtccacctgactccagggtctcatcttcgtgtgttgctagtgtgattacctggggatgagaaatcctgctgggggagttgaggttaagaggatgaggactccaggtgctgtggctcacgcctgtaatcccagcactttgggaggccaaggcaggtggatcaggagtttgaggtcaggagtttgagaccagcctggccaacatggtgaaaccctgtctctactaaaaatgcaaaaattagccaggtgtggtggcaggcgcctgtaatcccagctactcgggaggctgaggcaggagaatcacttgagcccgggaggtggaggttgcagtgagccgaacgaaattgagccacttcaccccagcctgggcaaaagagtgaaattccattcaaaaaaaaaaaaaaaaaaaaaaggatgaggactgggatgaactggtggctgggtgtggggaaaatggaagtgaaggaaggccaaaagagacagagaaggcctggcgcggcgactcacgcctataatcccagcactttgggaggctgagaagggggattgcttgaggccagaagttgaataccagtctgggcagcatagcaagaccctgcctctacaaaaaaaaaattttttttaattagccaggcttggtgacatgcatctgtagtctactcaagaagctgaggtgaggccaggcacggtggctcacgcctgtattcccagcactttgggaggtcaaggcgggtggatgacctgaggtcaggagttcaagaccagcctggccaacatggtgaaaccccatctgtataaaaatacaaaaattagctgggcatgatagcaggtgcctgtaattccagctactcaggaggctgaggtgggagaatctattgaacccgggagggggaggttgcagtgagccgagatcatgccattgcactccagcctgggcgacagagtgagactccttctcaaaacaaacaaacaaacaaacaaacaaaatacagaagctgaggcgggaggaacatttgaaccggattcggaggctgcagtgagctatgattgcaccactgcgctccagtctgtgtgacagtgagaccctgtctcttacacacacacacacacacacacacatgcacacacacagagagagagaaattagaagatactgaattggcagaagagaagggaaatagaaattaaaatactgaataggggagcagtgaacaggggatacccaaaagccaagagcgagagagagcctggcttccagaaatagtggagaagccaggagaactaggtgaaaacccagtgctgggttgccatcagcgagagctggagccatttccaacgaaccatcttgtcgtcttcacagctgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagtaagccaccccgctatctccccgacctaccaacccctctctcctggctccctaaagtcaccgcccccaggttgaatttcccagatctgtgatgcttgcaggacatgcatgtgtgggaggctgatgggaaactgtggcctgggtttgattatgagtcttgcaatcatccctccccctgtttctgctggagggcaggggacagctcttcctgaccacacccccacattgactatccccagaatacccagcaaaagcccccaaaaggagagtcagagaaatgagggaggtgggggcccaatcagtccacatctacttagggtcgccccatcagcacttccatccccaaccctttcaagtcaacatccaaacaaaagaaatcacttccaaggacggagcagctcaaagcgcagcttctagctggggttccaagaaagcagatttttcgaaatccttctgcagaaggaagcaaagagattttttgaaatctttctgcagaaggagaaggctggagctggggaactccagaattatagggaagcctcccaccacgctcatcccaaatttccggatgctataatgccaggcttggggaaagaggagaatttagttggttagctggtgcgtgctctcacttgcatcctctctcttcctcttttttttttttctcctctctctctggctcataaaaatggaggtaattagttgtgccctggtgagaagcagagagtgcacaaaggccccctgcttgagtcctcttcagggttagctctcagaaacacaatctgcagaacagatttttgttccaacatccttgcaggagaatttgcccttagcttcccccaccccagccaggctgaataaaattatgctgaaactactgtcttatttgaggaaagtaattagtcataggtgggagggggtggggagattgcagaagaatgttcatgaatattaggattttcagctctaaggggggactttgtaaacagctttagaagaagaaccaggccggctgggtgtggtggctcatgcctgtaatctcagcatttggggaggccaaggcgggcggatcacttgaggtcaggagtttgagaccagcctggccaacatggtgaaaccctgtctctattaaaaatacaaaaattagccagccgtggtagcgagcgcctatgatcccagctactccggaggctgaggccagagaatcacatgaacctgggaggtggaggctgcagtgagccgagatcacgccactgcactccagcctgggggacagagcaagaatctgtttcaaaaaaaaaaaaagaaaaataggaaggaaggaaggaaaggaaaggaaagaagagagagagaaagaaagagagagagagaaagaaagaaagaaagaaagaaagaaagaaagaaagaaagaaagaaagaaagaaagaaagaaaaagaaaggaaagaaagaacgaacgaaccaggcctccctctccaaccttcacctccgtccctattctggccacttgattcgggggacacctggtaggggatggggaaaggtgggagctgccagccagaggggaccccggcttgagcagcctcttgctgctatctgcaggttctccctggagactgaagtcgacctcaggaagcccctagagaacctgggaatgaccgacatgttcagacagtttcaggctgacttcacgagtctttcaggtaagaagactttcctttgcattttctcaccccagtggactgcgggggcccctaagaggaaaaaggaacctctccttgagagcggcagctgatctaatcctgtatccacatctgtttcagaccaagagcctctccacgtcgcgcaggcgctgcagaaagtgaagatcgaggtgaacgagagtggcacggtggcctcctcatccacaggtgagtctggctcaggtgaggctccacgggtgtcgcctccatcgcccttcaggataactggtccccagacccggaaaggaccccgcagccctctcggcacagagcagctctgtctgtgctcagccatcacccactccccacctgtttctcagcctggaaaacgggcttgggaccatggaaccctgtttcctcgcctgatggctcctaagttccctgactgtgaaaaggcctcctaaagaaaaacccaagttgttcccacagtgggaagtaaacttaagaaacatgcttatcaggctgggcatggtggctcccacctgtaatcccagcgctttgggggaccaaggcaggtggatcacttgaggtcaggaattcgagaccagcctgggcaacatggcaaaaccctatctctactaaaaatacaaaaattaggcaggcgtggtggcatgtgcctgtagtcccagctacttgggaggctgaggcaggagaatcacttgaatccaggaggcagaggttgcagtgagccgagatcacgctgctgcactccagcctgggcaatagagcatgactctgaagaaaagaaagaaagaaagagagagagagagaaaagaaagaaagaaagaaagaaagaaagaaagaaagaaagaaagaaagaaagaaagaaagagaaagaaagagaagaaaagaaaagaaagagcttatcaataagcccttaaaggatttagataaatgtgtgtaagggaagagctgatccattgctaccaagctcctggaggaaaccaggtctcagaggatgtccctaaacttttaaggttcatattcaggaaaacaaacaacttccagctgggcttagtggctcacacctgtaatcccagcactttgggaggccgaggcaggaggatcgcttgagcccaggaatttgagaccagcctgggcaatataatgagactgtgtctctacaaaaattagaaaaaaattagccaggcatggtggcatgcacctgtagccccagttacttgggagactgaggtgggaggatcacttgagcccatgagttcaaggctgcagtgagccatgaaggtgccactgcactcccacctgggcgacagagggagaccctgtctctaagaaaaacggcgggggtgggggtggtgccagtgccagcatccctctgttctaagacattgtcccttctcttgcagctgtcatagtctcagcccgcatggcccccgaggagatcatcatggacagacccttcctctttgtggtccggcacaaccccacaggtgagcctggaacccatcacgttccacatcctcccacccattctttctctcaggaactagtcccgacagatgcagacatccctctatccctgagagggctctgggcagggaacccataaccctaccctgcttcctgtcccaagaggaggctaccttctatcacccacagacagtgccgggtccccgctctgtgactcaggcagctgcgactccagacagctcactcatctgcctagatctcagtccttccacccacatccagcctgatgagctgtcccactccttctgcttctcaacccccatggttcttccaccctcaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagcctaatcagcccaccatgttctccactgaaaaatcctctttctttggggtttttctttctttcttttttgattttgcactggacggtgacgtcagccatgtacaggatccacaggggtggtgtcaaatgctattgaaattgtgttgaattgtatgctttttcacttttgataaataaacatgtaaaaatgtttcaaaaaaataataaaataaataaatacgaagaatatgtcaggacagtcactgccttcaccttctccatttcacaccggtggtacaagaaatcagaagcctaggccaggtgtggtggttcatgcctgtaatcccagcactttgggaagccgaggtgggtggatcacctaaggtcaggagtttgagaccagcctggacaacatggtgaaaccccgtctctactaaaaatacaaaaattagccgggcgtggtggctggcgcctgtaatcccagctactcgggaggctgaggcaggagaatcacttgaagccaggaggcagaggttgcagtgagctgagattgcatcactgaactccaggctgggtggcagagcgagactccctctcaaaaaacaacaactacaaagacaacaacaaacccagaatcaaaatcctgttggtccatagacctcatgggtggaagagaccttcctacatccaggttggcccaacatgggggagtccatgaaatggtcacctcagctctgccacaagccccaaggataagttgattctgcccctgggaatcatcctcaaaaaggaaaaaaatgttcccctgccataaactttccacttatgcagatgggcctgctcgtaagtcactgtcactgtgggttcccaactctgttcatgacacttccttccagcaccaaatgcttcccacccctctactcccactccccattcttcaaacccagctcaagttccagttcctccacctaggacttcccatggatccagccaatatcactctcaggtccggcgcagtggcccacgcctgtaatctcagcactttgggaggccggggcaggaagattgcttgaggccaggagtttcagaccagcctggacaacatagtgagactcttcctctaagaaaagaaagagagaaagagagagagaaagacagaggaaagagagagaaaaataaagaaagagagaaagaaagaaagaaaaagaaaaagaaagaaagaacgaaagaaagaaagaaagaaagaaagaaagaaagaaagaaagaaagaaagaaagaaagaaagaaagaaagaaagaaaaagaaaggaaggaaggaaggaaaggagggagggagggagggaaggaaggaaggaaggaaggaaggggatcaaaacatcatcactctcaactcgacactgactgagttttcttctccctggtctgtaacagtgcttggattctcgagtgttccttagctttgtgtgtgtgtgtcttaccctccccaagcccatcaaggtatcaggtttcttgaaaacaaggctctcttgttatatatactctagcatcttcttgaaaatggcttcctgaaaagtcttttattatctttctccagcatctcgaaggcttttggcagagggcacacttccctccattagtttctgttcaaatattcaaaaataattttttaaactgaaaaaagaccaataaccatctgttgaacttgtatactgtctaactccagaaccctgaagataggattcagagatgcagccccctgaacacccagctcatttctattgctttttaaacaattttttgtagagacggggttgcaccatgttttgaacttttgggctcaaacaatcctcctgcctcggcctcccaaattgctgggattacagttgtgagccaccatgcccagcttggctcaggtgtacctttcaacctccttataccctgaaagtatgactaattccaggccccaggctagtggctgaaaagttacctgtctctaacagatttgcttggaaagctaaacagcctctttttgcacaaaatgcttcaaaaagcagaacaaattagccctaactctgcagtatacagttcaaaaactgctcatccatgctgagatggctctagaggaaaaagagttcttcaaaaagccccctcaccacagaggaggcttgttccaggaaatgccccatttaacagactctcagctattgtgaggacaaaaatgatgtagactcacagcacgagacctgcccagccctgtttgactttgcctggccctgtcccttgcctgttctaagtttgtcttcccttctctatttttttttttcttttaagacagggtctcgctttgttgctgaggcaaaagtgcagtggcacaatcttagcttattacagcctccatctcctgggcccaaagccatcctcgcacctcagcctcccgagtagctgggaccacaggtgtgcgccgccaagccctgctaatatttgcattttttggagagatggtgtctcactatattgcccaggctggtctcgaattcctaggctcaagcaatccacctgtctcggcctcccaaagtactgtgatcacaaagtgctgggccaccatgcctggcccgctgttctttagggatgaaaaattaacaagatctgcctccagctaccgttattgtacatttcttgtaaacctcaaacagtggaattatttatctgctcatctgtgggcaaagagtatgaatgttgtaaatcccttaatggtaaaaatattcagtcaaaagcaaatttgtcaaggagacaccatctttttttgaaatcatttttttaggtggtttctcaagtaagctttgttgaaataatagtagcaagcggctgggcgtggtggctcaggcctgtaatcccagcactttaggaggccaaggtgggcggatcacctgaggtcaggagtttgagaccagcctggccaacaaggtgaaaccctgtctctactaaaaatacaaaaattaggccgggtgtggtggctcacacctgtaatcccagcactttgggaggccaaggcgggtggatcacgaggtcaggagttcaagaccagcctggccaggttggtgaaaccccgtctccactaaaaacacacaaaaaattaacgaggcgcagtggcgggtgcctgtagtcccagctactcaggaggctgaggcaggagaatggcgtgaaaccaggaggcagagcttgcaacgagccgagatcacgccactggactccagcctgggcaacagagtgagactccgtctcaaaaaaaaaaaaaaaaaaaaaagtagcaagcactcaggaaaactgagacacagagaggttgagtaacttgatctcattattatcattattttagagagggggtctctgttgtttaggctggagtgcagtggtgcaatcacagctcactgcagccttgaactgctgggctcaaacaatcctgtctcagcctcccaagcagctgggactacaggtgcgcaccactcactacccagctaattcgttttctttcttttttttttttttttaaaaaaaagtctcgctctgtcacccatgctggaatgcaatggcttgatctctctgctcactacaacctccgcctcccgggttcaagagattctcctttctcagcctcctgagtagctgggattacaggcatgtgccaccatgcccggttaatttttgtatttttagtagagatagggttttgccatgttggccaggctggtctcgagcttctgacctcaggtaatccacctgcctcagcctcccaaagtgctgagattacaggtgtgagccactgcacctggccccagctaattttttaattgtattttttgtagacacagggtctcactatgttgtccaagctggtctcgaactcctgggctcagcctcccaaagtgctgggattacaggcgtgagccactgcacccggccccagctaattttttaattttattttttgtagagacagggtctcactatgttgttcaagctggtcttgaactctggggctcagcctcccaaagtgctgggattacaagcatgagctactgtgcccagctgctgctatcatcattattacttaacagccacaggcagttgggcaccctgtgttccctgcctctacaccatattgcctctctctgtggcacgtgtgaacaggaacaggtctcccgaaacaccaagaagggctcagacgtaggcaggacgcggtggcgaatcacttgaggtcagtagtttgagaccagcctggccaacatggtacaatcccatctccattaaaaatacaaaaaaaaaaaaaaaaaaatagctgggcattgtggcgcacaactgtggtcccagctgctcaagaggctgaggcaggagaatcatttgaatccaggagacggaggctgcagtgaactgagatcgcaccactgcactccagcctgggcgatagagcaagacatcaagagagagagaaaaaaggaaagaatgaaagaaagagagaaagaaagagagaaagaaagaagaaagaaagaaagaaagaaagaaagaaagaaagaaagaaagaaagaaagaaagaaagagaaagaaagaaagaaagggaaaggaagggaaaggaaagggaaaggaaagaaaagaaagaaaatgaagagaaggaagggaaggaagggaaagaagaaagaagggaaagaaagaaagaagggaaagaaagaaagaagagaaagaaagagagaaaagaagggctcagacgtggtcgagtataaaggtgctggccaggcagtggctcatgcctgtaattcgagcactttggaaggccgaggcaagtgaatcacctgagtccaggagttcgagaccagcctgggcaacatggcaaaacctcgtctccacaaaaaaaaatacaaaaatgagctgggcgtggtggcgggcgccagtagtcccagctactctggaggctgaggtgggaggattgcctgagccaaggaggtcgaggttgcagtgagccaagatcacgccactacactccagcctggggtgacagagcaagaccccggctcaaaaaaaaaaaaaaaaaaagtgctgccctcacgtgccagtcatcagcagcttgtgtgttgccaccagccgcggggccagcctgaacacttgcttcctgaaccaggtctttgatctcctcgccaccagaggtcgctttcgctccagtggaaagctcttccctgaagcaaagcagctgtggcttccaccaaaatcttgccaagttggaactccattcaatttaacgagtatttatttaaaagctgacctttagactgggtactgtgaagcattaaaaagaaagtgtcagctattcttaatagcagatatctaatgacaagggacatcaaaaagctggggaaaaaaagctatgctgaccaggtgcagtggctcatgcctgtaatcccaacattttgagaggccaaggtgggaggatcacttgaggcagccaggagtttgggaccagcctggacaacatggcgaaacactcaactaaaaataaaaaaacttagccaggcgtggtggtgcatgccagttactctggaggctgaggccggaggatcacttgagccctgggaagtcgaggctgcagtaagctgtgattgcaccactacattcaagctggggcaacaaagtgagaccctgtcttgaaaaagaaaaaagaaaagaaaaggaagaaaagctatgcaagtcactgcaaaaataatggaaaggaacactgtataaatgatcgctatagtctgaaatagagtgtagactcatcagaggaggggctgtttgaaaccataaaggataggctgggcgcggtggctcacgcctgtaatcccagcactttgggaggccgaggcgggcagatcacgaggtcaggagatcgaaaccatcctggctaacacggtgaaaccccgtctctactacaaatacaaaaaattagccgggcgtggtggcgggcgcctgtagtcccagctactcaggaggttgaggcaggaggatggcgtgaacccgggaggcggagcttgcagtgagccgagattgtgccactgctctccaacctgggcgacacagcgagactccttctcaaaaaaaaaaaaaaaaaaaaaagaaaccataaaggataaatcagatgggtttggggtctatagggagggaatggcagggcattccagaaaacagcatgaacaaaatcacgaaggaaaacgtatagcagggcagatgcaagggacacgtgagaatgaactctcagatctccagctggtttcagcctgtcccctcctccctcctgataacccagccattcctctctaggccctcacctaatctagtccagcaagtcttccctggggcccttgaataagccacaatttcgtgttacccccctcatgtcccaggcttacagcttgcaaggctagaaaaaggcagtaacatcaaccacaatggaaaaccaagaggggataatttcaggggaaggaagtggcaacacgtttttggcaagtgaagtttgagatgacaatggaatacttactttatttcattttatttttaaaaaatggaaaccggctgggtgcagtggctcacacctgtaatcccagcactttgggagtccaagcatgtggatcacatgaggtcaggagtttgagaccagcctggccaacatggtgaaaccccgtctttactaaaaatacaaaaattagccgggcatggtggtgggcacctgtaaacccagctactcgggaggctgaggcaggagaattgcttgaaccctggaggcggaggctgcagtgagccgagatcacaccattgcactccagcttgggcgacagagtgaggctccaaaaaaaaaaaaaaaaaaaggaggaggaggagaaggggaaggaaaggaaggaaggaaggaaggagggaaggaaggaaggaagcaaggaaggaagggagggaggaaggaaggaaggaaggaaattgggcatttcagggaattgctggggcagccagagaaccaaattggagggaggtctcacactattgcactaggaacaatgccccacatcaaacctgtgcccctgcctgtcctgtggatgcaagacaccgctaatagcgccactccactgccaccctaggaacttgatcctctgtccttctgccagggtcttaactactttccatctcctcatgcccaccccagtctctcctggagcaaagtctggcaggtgcacagatgggcagagtccagctgacatgcccatgctatgcccagaaacaacgctgcaaaagtgaatatctggaaattgcaaatactactctgggaggttggtctatctgctccctaaattcataagacatgggattcccacactccaagaaggggggttagaacctgagtgctgaaaagaatgacaaacatcaaccacagagagtgttccagccgggtgtggtgtataggattacgtctgtaatcccagcactttgggaggccaaggcaggaagatcacttgagcccaggagtttgagaccagcctgggcaacatagcgagacctggtgtctgcaaacaatttttttttttttttttgagacagagcctcactctattgcccaagctggagtgcagtgtcgagatctcagatcattgcaacctctgcctcccgggttcaagcaattctcctgcttcagcctcccaaatagctgggattacaggtgtgcgccatcacgcccggctaatttttgtatttttagtagagacagggtttcaccatgttggccaggctggtcttgaactcctaacctcaagtgatccacccacctcggcctcccaaagtgctgggattataggtgtgaaccaccacacccagccctacaaacaaatttttaaaaatagctaggtatggtggtgcatgcctacagtctcagctacttggaaggctgaggcaggaggatcacttgagcccatgagtttgagggtgcagtgatcatgatcacaccactgcactccagcctgggcaagagagtgagaccctgactctaaaaaaattgaaaaaaaaaggtggggaggaacatggatcatagactcacaaagatgagaggcttcagaatccatctggccacctctcaaccaccgcaggaaccacctctacaaccacatgagaaaaggcatggggaggtgtcagtgtggtttgtaagatgacagtacaaacaccattcctaatagtgaagaagaatctcacctcttcagctaggtgcagtggctcaggcctgtaatcccagcactttgggaggccgaggcgggtggatcacttgaggtcaggagtttaaaaccagcctggccaacatggtgaaaccccgtctctactaaaaatacaaaagttagccaggtgtggtggcatgcgcctgtaatcccagctacgcaagaggctgaggcaggagaattgcttgtacccaggaggcggaggttgcagtgagctgagatcgcgccattgcactccagcctgggtgacagagcgagacgccatctcaaaaaaaaaaaagagaagaagaagaatctcacctcttccatgtttccatttatacaagtggcaccactaaccctccaggatttttctcccttcctctctacttagatgatgatcagcttcagatcaccagctcctttgaggcatctctcctgatttgtccctcgcatttcatcctggagtcaggattcccaaaactctcagacgatctttcagctttccaattgtcctttcaaattcttccacatatgactataagattaatctttttttttctgagacagggtatcgctctgtcacccaggctggagtgcagtggaaaaatcgactcactgcagcctctacctcctgcactcaagcgatcctcccacctcagcctcctaagtagctgggaccacaggcacgtgccaccatgcctggctaattttttttattttttctagaaacagggtcttcgttgcccaggctggtctcaaactcctaagctcaagcaattctgccacctcagcctcccaaagtgctgggcttacgagcctgagccaccatgtgcctggccaagattaactttattaacactgttttcaccaagttattcactggttcacacaaccataatgaccctaaactgccaactgcatcaaatctcaattcatctccatgcacaagctgtatcggatcaactaactcatagatcctccacattcatcattcactaacaatgaagaaatccaagttgggtgtcgtggctcacgcctgtaatcccagcactttgggaggccaaagcaggtggataacttgaggttaggagttcgagaccagcctggccaatatggtgaaaccctgtctctactaaaaatacaaaaaaattagctgggtgtgatgacgcatgcctatagccccagctgctcaggaggctgaggagggagaatcgcttgaacctggcaggcagaggttgcagtgagccgagatcataccactgcactccagcctgggagacagggcaagactctagtttcaaccacttgaattctatgactccctacaagcaaaacccatatttcttttcatttgtaatttctttaattttttgagacaggttcttgctctgtcatccaggttggagtgcagtggtaagatatcggctcactgcaacctccacctgccaggctcaagcgatcctcccacctcagcctcctgagtagctgggaccacaggtgtgcgccaccatgcccgactaatgtttgtatcttttgtagagacagggttttgccaggttgcccatgctggtcttgaattatttggctcctgcaatcctcccacctcagcctcccgaagtgctaggattacaggcgtgagccactgtgcctggctgcaaaacccacatttctaacaattctctcatgaagcctatcttttctgcagttaggcgtatcacattgaggcaaggaattttaaagagtgtcggctttccaagaagtgtaggagttgatatctctcgagcttccaaacttctgcaagctgctgcccggccctctgctttatttctttttcttttccttttttttttttttttttttgaggcagagtcttgctctttagcccaggctgaagtgcagtggcgtgatctctgctcactgcagcctctgcttcctgagttccagcaattctcctgcctcagcctcctaggtagctgggattacaggcacccgtatttttagtagagatgggtttcaccatgttgaccaggcctagtctcgaactcctgacctcaggtgatctgcccacctcggcctcccaaagtgctgggattacaggcctgagccaccacacccagcctgctttatttatctcaccactcataccactcattccttccttataccttctcatctctctcctaagcacatttcctttttttctttttttctttttttttgagacagagtcttgctctgtctggagtgaagtacaatgacacgatctaggcttactgcaacctctgcctctcgggttcaagtgattccctagcctcagtctcccgagcagctgggattataggcacccgccatcacccctagctaatttttttgtattttgtagagacagggtttcaccacgttggccaggctggtcttgaactcctgacctcaggtcatccacctgcctccgcctcccaaagttctgagattacaggcacgagccactgcgcccggccatacttctagtccttgctttcattccatatgatttatcctttctaatcctaacaacttgctttattgatcttattcttgacctacaaatgataccaggtgaaggggtgggttgccccccacacctgtgggtgtttctcgttaggtggaacgagagatttggaaaagaaaaagacacagagacaaagtatagagaaagaaataaggggacccagggaaccagcgttcagcatatggaggatcccgccagcctctgagttcccttagtatttattgatcattcatgggtgtttctccgagagggggatgtggcagggtcacaagacaatagtggggagagggtcagcagacaaacacgtgaacaaaggtctttgcatcatagacagggtaaagaatcaagtgctgtgcttttagatatgcatacacataaacgtctcaatgctttacaaagcagtattgctgcccgcatgtcccacctccagccctaagacggtttttccctatctcagtagatggaacgtacaatcgggttttataccaagacattccattgcccagggacgggcaggagacagatgccttcctcttgtctcaactgcaagaggcatgccttcctcttatactaatcctcctcagcacagaccctttacgggtgtcgggctgggggacagtcaggtctttcccttcccacgaggccatatttcagactatcccatggggagaaaccttggacaatacctggctttcctaggcagaggtccctgcggccttccgcagtgtttgtgtccctgggtacttgagattagggagtggtgatgactcttaaggagcatgctgccttcaagcatctgtttaacaaagcacatcttgcaccgcccttaatctatttaaccctgagtttgacacagcacatgtttcagagagcacggggttgggggtaaggtcatagattaacagaaactcaaggcagaagaatttgtcttagtacagaacaaaatggagtctcctatgtctacttctttctacacagacacagtaacaatctgatctgtcttgcttttccccacaaccaggatagtccagccagctaactgtctgtgccatcttattcttgacctaataaatagctgccgggcttggtggctcacacctgtaatcccagcattttgggaggccgaggcgggtggatcacgaggtcaggagttcgagaccagcctggccaacatagtgaaaccccgtctctactgaaaatacaaaaattagctgggcgtggtggcatgcacctataatcccagctacttaggaggccgaggcaggaaaatcacttgagcccaggaggtggaggttgcagtgagccgagatcacaccattgcagtccagcctgagcgacagtgcaagagtctgtctcaaaaaaaaaaaataaataaataaataaataagtagcaccacaatagttcaggcagccaataatctatgccactggtaagatattccactagtatatgtcatatatcttagacgatggtgttgaaaagcctaaggtggaggcagaggagaaaagaaaacaaaagcaaaaggaagaagaaaaagtaaaagaagaaagaagttgggcccgggcgcggtggctcacacctgtaatcccagaacttttgagaggccaaggcaggcagatcactcgaggtcaggagtttgagaccagcgtggccaacgtggtgaaaccccatctctactaaaaaaataaaacttagcggggtgtggtggtagatatctgtagtcccagctacttgggaggctgaggaaggagaattgcttgatccggggaggtggaggctgcagtgagccgagatcgtgccactgcactccagcgtgggcaacaagaacgagactccgtctcaacaacaacaacaacaacaaaaaaaaaaaaaaaaaaggaagagtctgggaagaggaaatgataggtaccattatgaagtgtttacagatacttgctatacattacctctaatcctcagaaccaccttgcaagatatgtactactgtcctctttttttttttttttttttgagacagaaggctgagtcaggaggctgaatcaggagaatcacttgaacccgggaggtggaggttgcagtgagccgagattgtgccactgcactccagcctgggcaacagagcaagactccatctcaaaaaaataaatacacaaaataaataaataaagtggagacaggaggtgaggccttaccaacaattgcatcatgtgctactcaaaaataatagcacattagttgccaacaattatctaccattaacttttctgcatgacaaaatttaagattttctcttatccttctatccagacctctttactttatcatttgtgctagggaagcaggagcctcagagagccagcataaagccattttaagttcagctccatcttgaaactaacaaggcacattccttgtcagtcacaacccaaagctgtttacagttggggaaacagcttaaagatacctgcaaggacagactcctccaactacagaaaatccagatgccccaatacccataacaatatgtgcctttaagataattatagttctggctgggtgtggaggctaaggcaggattgctttaggccaggagttcgagatcagcctggccaacattgcaaaaccccctctctactaaagatacaaaaattagctgggtgtcgtagcacatgcctacaatcccagtagtgagaccccatctctacaacaaaatatttttttaattaattggatgcactggttcatgcctgcagtcacagctacttgggaggctgaggtgaaaggatgtagctgtaatccagctattagggatgctgagacacaagaatggcttgaacccaggaggtggaggttgcagtgagctgagatcacgccactgcactacagcctgggcaacagatcaagactctgtctcaagaaaaaaaaaaaaggaagaaagataattaagttttgctttgatgtacttacctactagaatgtcaaggatagttaaattaacagaataataaattttgtcatgccattggcccatctgcacattgactcggcttagtttagtctctacaaaaacaagacccctatataagaaaaacttaaaggtcaggggcggtggctcatacctgtaatcccaacactttgggaggctgaggagtgtggataccttgagtccaggagtcccagaccagcttgggtagcatgacaataccctgtgtctacaaaacatacaactattagctggctgtggtggtgcatgcctgtagtcctagctacttgggagactgaagtgggaggatcgcttgagctcaggaggtggaggctgcagtgaactatgatcatgctactacactccagcctgggcaacagagtaagaccctgtctcaaaaaaagaagaaaaaaaaaaagaaagaaagaaaagaaaaacttaagacagtgtgttcctctgtttgctttctgaggacgccctactccgtaagggtgtagctttcaataaactctctcttctcatcgcactctacgacttgctttgggttccttcctgcatgagatccaagaattctctcttggggtctggatcgggacccctttttccagtaacatgtagacacacacacacattcagtttaaaagcccatattacaacgtacatccacctcatcttcacactgagatttgggtgacatctgtccctttttgagttttgacccaagttaaccttttaagacgctctttatttcttccccttttgcctcagttcacgttcctcccccttctccttacatgccttttatagcagcctctccttttctcttttattctttttctacacatttctcttactgtaaaatcagaagccaggcgcggtggctcacgcctgtaatcccagcagtttgggaaaccgaggcgggagggtctcttgaacccaagagttcgagaccagcctgggcaacatagtaagacccccgtctctactaaaaaaaaattaaaaattagctgggcatgacggtgtgcgtctgtagtcccagctactcaggaggctgaggcgggaggatcgcttgaggccaggagttcaaggctgcagagagtcacgatcccaccactgcactccaacctgggtgacgcggccagaccctgtctctaaaaagaaaaaaactagaggggtgtatctatcacgctttcaatagatattcagcttgggttctatacttttctcatgaccctttgtctttctgttttccaggtcactcctcctcccctacacttaattcccaccctgctcccttttcacggcccactccccttccaggagtcctgcctgctgcttcgaactccaatgatcgtcccattcacctctcaccccttttcccgcctttttgccccacaaggctccaaagcgcgaggaccttattgtcctcctcccccatcctccaaccagcccacaggacagccgcgttaccatgacgaccgggctcctagaagctgcagtcaaggacctggttgccatggtttccgcttctccgcctccagccccgcccgcgctccccgcggcgtcggcgcctgcgcagtgcgtggcgcgccgggagtcagagcctgggcgggacggggcggggcttcgggcgagagggagggcagcaggagaaaggataagaaagggagtgggactggcgcctacggtggccgaagtgggacgcgccgagccggaggctgcaggatggtaggctgtgcgaagagggaggggagggggaagcgaggggcgtgggctgcacctgcgggggtcctcgggggccgccctcggggtgaaccgccctgtcttccctctggtcctcagaggcctcccttgcctcggcgggtgggcgagccgggctcgggaggaagagggggaggtggcattcgggagaggggcggggagggggcgcatttgcagggcgagcgggggctgacgaaggggcggcggggccgtcccgagccgggaggggcgctctggtcttggcccctggggtccctcgatccccgctgcagggaagccgcccgaagcagagggttgcgggagtctctttgctgagggagggagcgggggtcaccagtcaccgagacccccttcccgggctgcacaggcgggagcggggacccgggtggggagaggggtgtgggaggggagtttccgggtcggaaaagggagcgccctactgcgttcgtggccgtgcttgcctagaggctgggcccaggtagggaagcgaagtggactcgtggtttttctccgagggacccaggctttgggatcccgggctccggggctctctgccccccttcccattctctttccggaatccaggccttccgtcttcctgcttgctccccacccccctatttgagcgccgcacgaggctccccagcgcccctcttttcccctccaccaaaaaaatcagttctcctgaattctgatttttcacttactaagctagtacagtgccacacacccagggattccaagcctgggcatgtccatgaatagaagagctactggggtaaggagaagactccaggatgtggcccagaccccccggaactgggaagctggggtacactgggcagtgagggttctgtcttccatcccacttgacccagcttgctgtctgttctgcagacattcttttaggggaaccccctgggtggcggggccctaactgagggaaattggtcacctggtgtctggggaggagtgggcgcttagcaggaggcaccctaaaaggaagaaaagttaaagagaaatgtcgaagaccaaatgcctgggtgcctgcagagaggaagataggatttgggcatctgcataccctgtgtggtgttggagatacatactacagaggaggatacccctgggcataggcctggcactggaggatgatatgtttgggagatcattaagatcttatggctatgatggggtggaaaattgaggtagcttctccagggacaaaataccagatttcagcatggtgatggatagagagagggtggaagagaacaggcatttctttggcgtcggttactgtcgtcttgtcactgtgctaggcattttacttacctttagaaagcagaccagaaagcaagaggtcagggaatcatggtaataatagtaataactaacggccgggcgcagtggctcatgccttaatgccagcactttgggaggcctaggtgggtggatcacctgaggtcgggagttcgagaccagggcctggccaacatggtgaaaccacatctctactaaaaatacaaatattagccgggcgtgatggcacgtgactgtaatcccagctactcaggaggctgaggcaggagaatcgctggaacttgggaggcggagattgcactgaacagtgattgcgccactgcactccagcctgggcgacagagagagactctgtctcaaaaataataataataatagctaacatgttatactgcctgcctgtgctagggacttgcatatatcaaatcatttagtctcacctaacaactgtgcaaggcaggtacaatgtccattttacacataaggaaactgaggcccagagacttacagtagtaattgggccaaggccttgcataactagtagagccaggatttgaacccagctgacagactcagaagctctgacttccccctgctggtggaaacacccagccttgagagcaaggggctggatgttggaagaaagtagagaagaattagctggagataagtttggggaatgtgtggttaccctcggttctgccctcccatcccccacagatgcggttcatgctattattcagccggcagggaaaactgcggctgcaaaaatggtacctggccacttcggacaaggaacggaagaagatggtgcgcgagctcatgcaggttgtcctggctcgaaagcccaagatgtgcagcttcctggagtggagggacctcaaagttgtctataagaggtgactccccacctactttcagacctgcctagattcaagttgggcagtgggttaaaatgtctgagaaatggtcgtcctgtaggtcagggagacctgggagctgaggacatctggaggacagtaatgaatgtcagcttccttttttttttttttttttctccccaaactcttaaagaagttcttgtttaccacttggttttgctcacgtgcaacacctgcctcactcacgtgtccctttgggccctgtcttgccccactgcgcatgtcccttgcagtcaaaggtcctgccctacttttcctaacacacacgcacacacacaccctttccggttccttctcctcggagcaggtaaattacttggctcaggtttttgctgagtcaaacttaggaggtcagagggcaggaaggaaaaagcatgcgttctttgcatgtttttaagacagcacaaagaggcaggtgagttactctttacatgccctagggccacctcacctgtggccagccagggcacagaccacctccaggctggtgagaggtttgagaccagaatgcctgggtcctgaagcaagcttgtagcgatgtctcatgcgctcctctccgcagatatgccagcctctacttctgctgcgccatcgagggccaagacaatgagctcatcacactggagctgatccaccgatacgtggagctcttagacaaatactttggcagtgtaagtctcctctgcccaccagtttccattcccagcaatcccctttggtaatccaccaaactttgaggcccctccagtctctgccctggaagtttcaggggagaggtgaagttggagcagtaagaacagtaattaatttccctttttaagctttaagcctattaaccatgtcccataatcgtccctgagtggggaaactgggggcaggatcaggcacttaatctccacttagagcttagctcggacttttcacacagagaccaagttcagatgttttgattagttcaaatttttatttttttagagacacagtctcaccctatcacctggaggaatgccgcctatggctggagtgcagtggcacaatcgtagctcactgcagccttaacctctgggctcaagccatcctcctgcctcagtcccctgaggaggtgggactgcaggtgcacaccactatgcccagctaatttttgtatttttttttttaagagatgaggtcttgctatgttgcccaggctgggtctctaactctggggctcaagtgatcctcccacctcagcctcccaaagtctgggattataggcgtgagccactgtacccagccccatattcagatttttatcactggttgctgatcttggatttgcattggagttttctggagaatttttttgaatggcaatgtccaggttccactcccagagattctgaattaacttatctgagggacagctcagatcttagatggggcgtagtagtaactccataatttgtgttataaagtgcttttgcgtcctctcgtttgctccttatcccatcctagcagtaggtgttggtatccccatttttgaggccagaaaactgagacttagagatgtttcatgacttgtttagctagatggttagtggcagagccctgcttggaactcaagtcttccaccccatggtccagaattccctctttccctgccccacagtgtccaggaagcggggctaccacagcctttggtgggaagaacgtcacagattttggggggaaggcaaggggaaccatagacaaaggtagataaggcaactaatgtttcatgatcaggaggaaagatgcaagaggaagagggaaaaaaactgatccaaggcaaggcgcggtggctcacgcctgtgatcccagcattttgggaagctgaggcaggtgggtcacttgaggccagaagttcgagaccagcctggccaacatggcaagaccctatctctacaaaatattttaaaaatgaggtgggcgtgatagcatgcacctgtagtcccagctactcaggaagctgaggcaggaggatcacttgagcccaggagtttgaggctgcagtgagctgtgatcatgccactgcactgcagcctggacgacagagcaagagaccctgtctctaacaaaataacgaaacaaaaaaactgaaaaagctgaccaatgactaaggcttggaggttgggggcagaacccaaggtgggaggcacggtcttgaaaaggaagtggctccagttgcccctccatgctcaacttagcctctccccgccctcccaggtgtgcgagctggacatcatcttcaactttgagaaggcctacttcatcctggatgagtttttgatggggggggatgtccaggacacctccaagaagagtgtgctgaaagccatcgagcaggctgacctactgcaagaggtacgggccagggacagtgaggaggaggaggaagcgcacagtggcggacagggtcctccctcccctggacacccggcctgccctgcccaccgtcgccaaggagccccccctccctgtggtatctgacccccaccacgcccagctctccagcttactctcaagccctgaccccatggggtgggaggtagggtggcagatagcccacctgtgcacccctcattccaagctctgtccttctcccaagcccccagatgtctcgtggccacccagtctcctttgtccaattctctgagcaaggcttaacaccaaagggttaagggtttgctctggagttaagggttgcttggagaattggagaaggggcacctggtaggtcattcttttcttccctgatttcttcccccacctctcttttttttttcttttttttttttttaacacacacatactccaaagcctctgcgtctcaggaggaaaaggctatggggatttgggggctggcagagggcacctggatgttggttctccctgctccccagcgcccccgccctcaccccattactgcctgccccctctctgctgtgccatcaccgagccctggtgcagggagatgagtcaccggaatctgcagtgatgagcgagcccggcctgagctggctctgaggagggggagagatggggggcgagggagggctcatctttgcagtgggagagagggcagaactgaacttgccacctcactggtcgagcttcaccttcccacacacgcacacacacacaccctggcgcgcacacacacacacacacacacatgccctggcacacacacccatcgtgtcaccagtgttgcgctcagaccctaatgacgcatctccctgcagcctggttcaccttccctcagggacctggcactcacctccctctgccttttccccactctgaaaaacaggacccagtcagctgtaccccccagggccccagaaagatcccccacctccttgtcccggcttcactccctttacccccaggtgtcaaggcggggcccctgcttaccaaggtcctggctttgggttctctgggtggtgacggactcaacctggaccccccctgccactgttggctggctctggggctggccctgggaggctggtgggtgccccatgaagggctcactggtccggccttgggctcacattggcttctctccccctcccccgtgtctgtgcctcccccgtctgactcttcctcgtgactgctgtcttgcccaccctgtcggcctctcctggtgtctctgcgtcaccctctgtctgtctctgccttaggaggatgagtcgccacggagtgtgctggaggagatgggtttggcatagcccctgctgggccggggtgtggcgatggggtcctggcagcgtggcgggaacggctgcttctcctctgcccagggccctgttcttggtgggactcggctgcccctcctctgctgcctcacctttcggagtgagctgtgggctcaggcccttcaaacattccctccctccaccccctacctccactttccccttttcccactgaaggttttagaagctaggaggcaggaaaatgtgacccagatgggggtgctatttggcttttattccctgcctttgcagaactgatgtcaccccagatgtccttccctccctaataactgtaaatatataaatatgtcaggttaaagggaaaaggtgttcagggcacttcttgtcctctctgtcccataacctacctccaccctccccctagccagccaggcagcttctctgcctgggaggggagcctggacccccctctttctccttggctgcagtggggcctttatccagtgccagggaggaacaacatagttaatttttttctaaccttgccactttgagggaaggaagggttgggggaagggcaagctttatgggaccctggtcctggccctggcctttcactccagttctgggtgaggcaggagctgggaggggtggggagggggagggggaagtgtctgcctttatgtcctttcttctgaaataaaagtaaaagcatttctggactttgctggccttgggctctgtgaaggaaggcagggctgctggcatcatcgggaggccaaggggatcctgccagccttgggaagaaacggagagcacgcagttaaatgagaattccagatacacaacaaatcattttagggataaatgtgttgcatgcagtgtttgggacacatttaactctaaaactgcatctgttgtgtatctggaatttcagatttcatttcctgtattttatctggccaccctaccttaaacggatttttatgcccaatgcgtctccaaacgggctgcccccaactccctgagctgcctttatggaaggaggagcaactcttcaggatgcgggtctgttgtgggtgatttcgtttgcccagtttagttttatttgaatcagttcacaagtacctgcgattagaaaaggcttaagatgggaggccaaggtggggaggtcgcttgagcccaggagttccagaccagctgagcaagctggcaaaacctcatccctacaaaaaatgcaaacattagctgggcataatggtgcacacttgtagtcccagctactcaggaggttggggtaggaggatcacttgagcccaggaagtcgaggttgcagtgagctgtgattgcaccactgcactccagcctgggcaacatagtgagaccctgtctcaaaaaaaaaaaaaaaaaaaaagttaagaagtggctttagccagcatgtctctgtggctgccactccctcctccccgcatttcttgcaaagtacctaggaccaggaatgtagggatcagtggagggaggtgggtgggtgaacacaggaaactccggcattacccgcgcggccccaggatcttgggaaaaaggagcccactggcacccagagttgtggcaccttctgcgggtcgcctttttcttttgaaccagaggtcgctggactgatcgcagacaattggctcttttattgcattccgctgggcctctctctgtcaagccctggggaaagatgactttaccaccagcccccaagctggaagtggaatttcgggtttcttaatgggtgcgagccacattcttctcctctaaaaacagtttgaggactttccccacttaccccttgccttagtttgggggcaggaagcaggaggagacggggactgtggctggcgcttccctcaccgccccttgggaaggaccacacaggcccaaggggacgggtgaataaccgagaacaaagtcctaataaaatagtcacttttatttcttagcaaaactatttcctccgtgaggggtatttacaacagagaaaggaaagaaggggtcaattcacagcgacttggagaggctggaggggctcgtgggaggcccgaagggctggagacagacacacttcacacaattaactggaactgctttttccggtttccgacggggacgtccccagagggacttgatggggccggggccccgcgtggcaagggaactcgcacaaaccacccgccctcctgggctggcccccggtcacccgcgggtgagctctgggagttcgggctcaggacccgggaggggggtctggcaggtcccgacgcagcccggggacaggggcagggccaaggggcagggccggggcgcatgcaaacaccgagggggagcgggcaacacggaggggggcgccggcgcgcgcgcgcggcgggggcgcgcgggggcgggaccgggaagggcagtcacgggcgccggagcagcacgtgctcgatgtaattctccagctcctcctgctcctgcagccggcgctcctccgcctccgcctcctcctgcgcgcgccgcgcctgggcctcccggccgggatagtggcgcgaaggcggcaaggcgtggtggtagtggcggcggcgcaaggccgagggcggctgcagtgtccgcggccggatgtagttggggaaagggtggtacggccctggcgggtacacctcgtcctcctcccgatcccagggcgggagcacctcgttccagtccggcagctcgtctcgtgcgggagcgggggcggggggcgggggctgcggggagcggacgtgggtgggggcgggggcggcacgggggggcggcacgggctcgggaggggcgttcttcttccgcttccgcttctcctccacctcctcgatgatgctgaccacgtcgtccgctggcaggtggagtttggtggacagctcaatgaggctgtcgatcgtctgcggatccatctcctcgtcgtcctcctcctccccgccctcctctgtcccctcggcctccttccgccggtggcccggcgtctcctcctgggagcgcttgtcctcggcgccggcttccccgtcctcctcctccgcgaacaggagcgcgttctgccgcgccctctccgcctcctccgcctctgcctccgcctcggccgcctcctcatcctcttcccccaccctctcctccccgccgcgtctctcctgctccgcctcctcctcctcccttgcactctctcgctcctccgccgcctcctgcagcccccgacccccgaggccgcgctgccgggccccgccctgcagcaaatactggagcagcaggtccgaggcgaggtcggccagccgctcttcctgcgccgcggcctgccgcgtggcctccgcctgccgccgcccggcctccacctgcgccagcccttgctggagaaggcgctcgcccgcctcggagccgcccaggagtgcgctctccggccggcgcgccttggggaacggggcggccacgccttggtacgccttggacaggggtgccaatgcctcgcctaggtgtgttttgggggaggacactccttccccgaacttgtgggtttcgggaaggggcccgctgtcgggcatacgcgcctggaattgagagggggccgggggcggcaggggcgcgcgctccgggacacgcgcctggaactctccccaggaagcgcgccatacgcgctctggccccgggctctccagattcactcgggtcagcgtgtgcgtgcgggtttccgtctctgctgccgccgtctcctgctggcgcttggcgctacttggactgaaatctcgcagttcctggagcagggacgctagcgcctcgagctcctcggagggatcgctcgcctcgggcccattctccggagtctgagggcgaggcggagccgcgggctccgggctctccggcgccgggaggctgtgggtctggctgcgcacggtctcggtcagcagagcttcagctgcttcttcctccggcccctgctgggagccgcttggtgccgggggtgaggcgggacggtcgagtgcctgcagcagcaccgcggccagcgcccggggatccacgccctggaaaagctctccctcgtcctgcggctcggaattccgagcgcctcggacctctggggcgctgccatcctttggcccgggcactgcgtccccggctaccggctctttatgctcagagctgagaggaggaggctgcgcctcagggcgaccagggggtgctgcccctaacccgttgatcagcagaaggcagaagagggcggaagccgacaatctgagggctttcatgaccaagaggctgccggagactgaaaaatagaagggaccaaaaaaagaggatttctgtaagaattcccctctctaggtctacgttcccttcccccacctttcagcaggaaacccggggcttccctgatcccccaaaccttacccagaagaggaacgtttaggagcaaaggaggaagatgttgtgccgatctctggagccgtgtaaatgggaaaataacccccgaagccctcgcttacccagggcctcctcggccccttccctgagccatctcactgccatcgcccacgtactaagcagcagcaatatgggggaaaaaaaaatctctgctccctccccgtaggactccccggatggtgcgtgggcgacttcagcagcaagagaaaaagctgcgactcccccgtttaccggggacccttaccagggggctccccgctgtgtttgcaacacccccatcgagaggagagagaaccctccctcacgcccacggctggctgccgagggtttgagggacgaacagcggagtatttaccagctggtgtcacgacgcgagaggtggagaggagggtcggggcagggtggggacaggggaagatcgggacgcgtccgcctcggctccgagcggtggccggggtaggagcgacggtcgaggtctggcgtcccgtgggctgggctcagctgggtcggcgcggctccgggcggctagctcgctccggcttcagcacgctggacagcgcccgcgcctccaccgccttataaaggggagcgcgcggggtcacgtgggcgcgccccgccccattgacgtcaatgttcattcatggggaagcgggcggccgcccggaggaggaaggacgcccgacgattggagggtgcctgcctctcccggcctgcgcctgcgcgctggggccttcggctgaaggggtgtgcgctagcggagctccgggaaatgaatgaatgaatgaatgaatgaaatgctgaagcgggcagggcggggggctatgaatgaatgaaggagggacgaggagaaaaggatgaatgaatgaatgggagaatgaatggaagggtggatgcagaggcgctggaaggcgggagggtgggttgcagagcaacttggagctgctggatggagcccgcgttgctgagtggaatagaaaaagggtagtcccagcgtctgactgggcaccagtgggagctcaatactggggaggggaaggataaagattcaagagccgaccagggggccgggttccccaccccgagaccacgcgcccaaggacggcgtgaacctgagtgcagagggacagatctgcacgcctgcgggcgctgtccgcgaatcggcagtgcgggtggcagccccctgacaggggcagatgggtggatcttgcggttttggtaatcccatcgggaagggtccgagtcggaagagaaaagggaaagaatgaggatgtgtgtgcctctgggtcgtgtctgtttcacgatccgtgctgtgtctggagaaaaaggtgtgtctgtccttctgtcccctgactgccgcttgtgtcaaggccccccgctgtgcccccttcggcccatctcttcccacactcccccagtcgctttccctcctgccgctcccctcccccaaggacgtttggagggcgagaccagagcaggtggggagggggttgcgcaggtggggggggggtgactcaactccagctctctccaccgcttaaagggaaggtttgaacgcgatgggagggggacctgcaacgtcagagatagcccctctctgccaccagccccttcgaccgtatctcctgccagagaggttggggctgggggcttggacagctagaggttccgggggtggggggcgatgacgcacagatttgcgcaaagagaatccatcagagagctgggtccggtgaagggagaccagccgtgggcgggggtgggggggaggacggaaatggggagggggcaggggagaggagagggtggcaaggggggcagccactgcaggaatgaaggggagggagggaggaagcctggagggggctgcggtgctggcagagagcggtgcgggggcgtgggcagggaaggggggaatgacaatgacacacgccagacacacacgaactgcggacataggggtgctcgtgggcacacgcagggcacccgcaccctgagactctggaaaccaaggtcagggctgtctgtcagggtcttagagactctttcagaaacggtcctcgcccccctcctcacctcttcctggcactgaccctccttctaacctccccctccctacacacacactcacgtgctttctctctataaccagcagtggcttctcgcttccatcagtggctcgactttcccagaagaaagcccatcagctctgcggggaaagaaatggggaggagtggagagggagaaaagagagaagagatgggggcaggggaggaggaaggaatgaagggatctggaacaggtagaaggagcaggctaaaggagaaaaaaaaactgagaaccaaggtgacagtggagattccaaagaaaggagggaaaagagggggtaggagaccccgaggcgatgcctcccatccccacgcacaactaaccacggcctcccctccttcctctccccgcccccatgtccccttcatcttcccagcctggatacagagagcagtgggatggggagggggtcacgttaccttcctgaggaggatgaatcagagagagatggctcagacatctggagagggggaggggaagccatccaagagagaagggagaggggagggagggagccaagggggctgactgagtggctgatggggatcagagaccaagttgcaagatgagagaagacgggcacctggagaaactgtatttgcctgttggcttgtgtatgcacacacacctgcgtgtacatgtacatggcttgtgcatgtgtgtttatgtgtgtatggagatgaatgagatacatggccggggcggggagaaaaagaaagggaatggggggaagcaattgtgtatttaatcagatcaagagtggatgggcatggtagtgtttccaatttaactatgtgatgttctattccagtctgtgtggctgtgtttgtgtttgcttccaggtgtgggtgtgtgtgcacgcgcgcgtgtgtgtgtgctgggttgtttttttttgttttttttttttatctcttctgcatgcctgtatgcccatgcagtttggagtctttgagcacacccagttctgggcatccgctggtgtccaaaaagtgtgcctggcttggtggtacacaacaatgtgtgtgtaagcacacacatgcccccactccacatccctcccttcctccacttaagctcacaagcccaggagagtttctcagaaatcagcatccccaaggctccagtcgtcctgtcgtgagggagcggggtggatatgaatgcctggggcacaaaaggcttggagattccacaatctgggtcttgactctgtctgcatggtctctggactcattctgtttctgcctctctccatcccagttccctacctgggtcagctcattcacacactactcgcctctactgggggtgagctgtgcccttcataccctgctgcctctaggggcttctggactgggcacaggttcttgggatagggaaaggagggagacagtagaaaaagaaagagaagtgaagagactgggagagacagagtgctggaaaaggcagagcccatatggggacctagttggggatgcctggtattggttctgacttgtttgtgtgaatccatcatctctctttgtatttgtttctaaatctccctccaagagggtagctggagaagatggggtccagcatcgtagggttccaagctgaagatgtggaaaaccaggagagtggggaagagcactgggtcttttccctgagaaggtctgggagagaaagagagagcaggggcttgtggaccccgaagctcaccggtcccctcttctcctccccctccccccaaccggtgactcacctctgcagcctttaactgcgtcagccatggtggaggggcaggcagggagggggcactttatgagaggggaaggggtctccactttgaacttacagaagatgctgaaggccccctgaaaattgccccatttagaacacactcttgccctatcctctccccatcccagagctggggaatcctggggaagggactgaggggtgggctgggggctacgggagggtctggaatgatgagtgatgggaagggggtgcccaccctgatttttagtcctgaggtcccattcgccctcagacccccacccactcctgagtgggggaggggagacccagcaagcgggggagggcaacgccgcagagatgagtcaccaagaaagagagagagaaagagacagagatggggagagacacgaagagagagccactggagagactaacacacacacacacacacacactcaaatacacacacagacacacacacaccatcaaggtggtatccaccaagagaaagaaaaccacttctgtcatcctgaaccagcctccttcaacttccacctgtcacacacataacacactgtcctacagaatcacactgagattcggagacacacccacttacacaaacacaatgtcacacctagcctagcagttacccagacctaggctggagatgtgtgcagggcaatggaggtgtgctctaagtgttctcactcattccttttaccaggtggagggaggggcgccaaaaccctcaaggaatgcgacttcagagacccaagacgcggttgcagggactcacaggctgcgctgtgcacactcgacaatacacacaagtcgcctgcctccccgtccacacgcacatagtaacatatcaagcatcgtcctcatcaaaaagcaacagaaaaaaaaccacttgaggtacctgattctggggtacaccaggcacgttgtcccgaatccatcccacccagacaccccctgactcgtacacacatgtacagaaacgcagacacacaaagacgcccacggacacactcacacggacgaaaaccaccttgtgctgccttgcccgcgtccgcgcacgtcctggcgcccccgtgtgttactttgggggatcgcagtctccgccaatccagagcaggcttcgcgggggcggtgcccaggctctctccacctcctccacccaccacgggttgcccacctagaaaaggggaaagcgataccggctggccacgggcaagtctggaactgggggttagatgcctggatcctggctgatggaagctggtcttggatgccaagtgggacttgggagaagaactcacagaatttgggtagcagccgactcccaggtccagggaaaagcgaggcccagggcgagtcaggaggccgtgctgctgcccacctcggtcctgagcagcggcttctttcctccgcttggcaggggtggggtaggggcgcacagccagcagccctcctcagaacccctctccacctcccccctctccccgggaagtcattagccccattgccacagggcttagagatcctctctgatacaggctgctggggaaaggggaagataatgtgtctcatttctccccattgggaagtgaaacagacacagagggtggccaaaaggtccattttattgtcatacaatcgtggtcaccgtgacaaggagggtggcagcgtggccaggcacaggatgcacccaaggagagcagcacagtagcaaaaccagagctgcggaaagtggcgaagcggcggctgcccctaccccccaccttgtccacattcttccacctgctggtgtccacctctgcccacatctcggatcccatcccagcgaaccccaaactcactgtcaacattctcctagcacctacgcattgcccaacaagaagttcaagctaacatcatcccacagagtgaagttctaggtcatgctctaggcttgggacacccgggttctattcgctgggaggctcctagctactttgaatgacttagaagtcactttgatctttcccagccttctgttttctccctctgcctttgatgtggggaaccagtattggagacagcgctgtgcagatgcagctgttactacttgcaattaaatagaagactctcaaagttctaaaagcataaatgatccagagaaagggagtggtgtccagaaataccaggaggtgggaattccagttgcactagaaggcggcctggttggaaccaggagacagcccctatgccaggctgagggacccgccctccactgtaccaaactcttcagctagaaccacaccacacacacaccaccaccaccaccaccaccccgcccggctcgggtgagctggggcacgggggagcctgcgaaatagcaaagttgtgcaaagcaggaggaaaccccagtgagagaggcaggctcagaaagtgcatcaccagatgaacaaggacccaaatgggggagcattctaaatacatttcattttacagtatcactctctctctctctggattgcacactgggctggtgatgggaaggaaaacagggacagaaaaatgggcactgcggtgttaagcgtcgtagagatccagcgaggatgaggatgaggggctagagagaagtggggagagggcactgatctctggcaccagaagcttgagcagggggaggggattggggtgtggagggcaatgcaggaccagtttttagggctttagaaagggcagaaggaagaaaagaaacgcggattgggcggctgcgctcgggaacgaggctgggcatcccacacactcctgagttcttccacccacgagggggcagaagggagaagccaggaaagggggagttcacgccccgggtgaggtgatggagagggaaggaaggactctgaagcctgtgcggggaccagttctttggaaaaacagagggtggataacagcagctcaaggcccccacccccagcccccacctaatagtccgcaagttcaggtcagggagtgggcaatggtgtttgggggagctagaggcaagatagtaagggcaaaagggacagagtggggggacctgcgccggtaagggcaggagggcaggagtcagaggggacttcccaggagacgcagcgtcggaaatggcaggaaagaggctggctggggaaactgcggaagggggcgggtctttttccccctccccgccagaggagaggcctcagatgtcggcctccagcaggtactgttcagtataacccttcaccagctccagtcccagcccgacctggcagaagtcagccagctgtgaccacagctggggctccaggaagagctggcacatgacgttgaggccaacgaggcgcggggcctggcgctgcaggtgccacagcagctggctctgcacctgcgcgccgtcgctaccgaaggcgtcgatgttgagatagcgccaagtgccgtcccctccgtgcgggatggggaaggggcgcgtgcacagcgtgagcacgcgcgggcgcgcgcggctgtccacgcgccactccaggcccttggccgccagcaggcgcaggttgctttcgctaggccggtagggctgccagtcctggatgatggtcccgcctggaagcacgtcgcgctgcacggagggtgacagcaggaggcgtgccacgtcgccgcctgcctcggacacggcctcggtgggcagcggcgagaaggtgccagaggtccgcagcgccgccagccgcgcgcccagcccggccagcagcgcggacgcgttgaatcggaccaaaaggatgccctgcggggggcacgagtgagcgcggggagggggggcgcagcagggctggcgagcccggcgcctctcggcaggcatcttcactcccacgttcacgcccacgggctcccacctgggtccctctggaggagcgaccgtgagggctccgggccgagtggggtatgggaaagcctgggtttcaagggtaccagacagggaccaggggcacgggctcccagtacgtggatccccaagaagaggtgggatcagtatggagtagccagggaactgagggtacgggtccccgggtctccagggacgtgaaagccagggaggccgatcagtcagtaggattgggaggagcagagggaggacaaaggctcagaggagatggcttggggccctgaacagaggcaggatcctaggggaggcagggtccccgaagttgggaagtaaggactagggtttcaggctgggagtcgggagtttgacttcccctgggtcaaagggtctggtaaggactgggagtcccagggcaaggctcatgggccgcgccactcggtaaagcccagagagagagcaagcccgtgttctgctgggggtggggagggtctccccatatgccccagcagagaggcccagccatccgagctccctgtccttctcacctgcttggtgattaggcggtatttcttcagctcccgggggcccagctggtcgtcccgggtgagccgtgccaccttgacccccgggtgctgtctcttgaccagctgcgagcagaagcgctgcagcagcccggccacgcccttcccgcgctcccagggcgccacgcgcagcccctccaccagcaccgtctccccggcgtcgatcacgttcaccgactccagcgcgatctgcggaccgaggccgttagcgcggggccctccccgcctgcgcccctgccagggctgtcggttcactgggctctcctcttcccacacgctgagcactccgcgtcaccacccgggcaccaggccaggcctggggtagcacggcacttgggcctcagattacagagccggcagaacagtcattcgccggggcttcgtccacctttttatttttatttattttaaatttaggacagggtctcctcacccaggctggagcgcagtggtgctatcacagctcactgcagcctcaaactcctgggctcaagcgatcccctacacccccaagacttcctagtagctgggacaacaggcacgcacccctactctcggctttttatttatttttattttttggagacaggggtctcgcatgttgcccaggctggtctggaactcctagcctcaagtgatcctcctgcctcggcctcccaaagagctggggttacaggagttagccgacgcgcccggccccttccacctcttgagatgctccagctccgagtgtgtcaagttccggaccgctcaccacgccccttccttggctcctcccccgggagtttgccacgccccacttcctcttagctcctcccccagcccaaatctaagctcttcctgctcccacccctagcctaccctctctctcctgctccccacctcctttgcttttacccccagaaattcctccggtggatcctcgaactttcaaacgctcacctgtccctctggttcctctgctttaggagtcctccaccccaccctcactgacatagcccctggatccgcactgcacgccctgcactttggtcccaaagcaggctctatttccgcagcctccagaggaaggctggggagaagctctcatcctcctcttcctaagatccacgcacccgcaggtggcttcaccccatgtcacctgggccgtgcccccgggctcaccacgcctccgttgcgcttggccagcaccaccgtgcggtcggggtcccggagccagctgtggtagcggctaggaaggtagtccaggccgccgtagatgccccccgagatggccagcacttcctcaaactcccgttccgtggccaccacgaagtccaatggctcggcctcagcctcaggccccgatcccgacctgggctcggcctccacctcctgtggccgggtttcagagcttggctctgcatctcgggcagtcttcttttccggcttagggacctctgaggtggctgtgccacagctggcttccagcttcatgaccctgcagaaaaaaagagaattcagcacctggatcagcccctttgtttccacatctgggcccctgcacacaaacggtcctaatgccccttcctacacaaatagcctttctttttcttttcttttatttatttacttatttacttatttatttatttattttttgagacggagtctcgttctgccgcccaggctggagtgcagtggcgcgatctcggctcgctgcaacctctgcctccccgcttcaaactattctcctgccttcacctcctgagttgctgggactacaggtacgtgccaccatgcctggctaatttttgtatttttagtagagacggagcttcaccatgttgtccaggctgatcttgaattcctgacttcaggcgatcctcctacctcagcttcccaaagtgctgggattacaggtgtgagccaccacattggcccctttcttcatttcttcttcttattttttttttaatacagatagggatctcactactttgcccaggctggtctcgaactcctgggttcgagatcctcctaccttggactcccaaagtgctgggattacaggtgtgagccactctacctggcccacaccctttcttttctgctgtccacacagaccctgggagtggaggtctcccagctccctccctcttcccaccagctttcagaaatctcagctctacccaccccactcttatgggatgctccctaacagtcagaaacttagaaccctgtgagatccccatagaccagccccagatccagggacccaggcaaaagaaaaaaggcacttgggggtagttttaaaaaaaaaaaagaagaagaaggctggacacggaaactcatgcctgtactcccagcactttgggaggccaaggcaggtggactggttgagtcatggagtttaagaacagcctgggcaatatagtgagactctgtctataaaaaaaattagccaggcatggtggtgcagtgggcctgtagtcccagctgctctgaggctgaagtgggaggatcgtttgagccaaggagacaaaggttgcagtgagccgggatcacaccaccgcactccagcctgtgtgacagagagagaacctgtgtcaaaaaaaaaaaaaaagaggcacactccttcaaggatcccatcacctctctaagacctctgcaaggacaacatgagaaagaaactcccactgtcacccctctgtccacaagaaagttcccagagaacagtggagggctcaccctcaccccatttcctgccactaccagctacaatcccagccagggccaagggggagggtgtagaccccaaatatcctccatattgctgccccagttccctgtccccccacctccacctcttgacatacacattcaaatataaacaagatagatagctccactagaaaatgtcaagccagcctagcatggcggctcagccgaggcgggtagatcacctgaggtcaggagttcgagaccagcctggccagcgtgatgaaaccccatctctaccaaaaatacaaaaattagccaagcatggtggtggacacctgtaatcccagctactagggaggctgaggcacaaaagtcacttgaacccgggaggtagaggttgtagtgagcctgagccgaggttgagccactgcactccagcctgggcaacagagtgagactctgtctcaagaaaaaaaaaaaaaaagaagaagaaaatgtcaaaccattctttctaagccaatcaatgccccccgcccccgcccctttccttccttctgcccctcttccccatagggagacttgggctctgtccactccaggactaggaattgtttgaaaagtgttctgtcggagctccaccaaaatgaaagagaaagccccctatccaagtcccagcaacttctatttctgggcctgtctcttccagatcagccaaatcaaccaatcaaatcccccctctctctttcagccaatgataatgctcacctcagataacctacgttaagcctcattaaccaacatcccaagggcacacagaccagctattctgtgggggtgatattgacttattgacttaactggaatttttatttttttttttaacaaaagtcttacctaggtttggggaaggttgaccagcagtgggaccaaggagtggtttgcacttccaagccataaacagagcttgcctaaacgcggtatgcttgctatacaaaccaagcttcaagacaagagttctatctccctctttgttttctgattctttctcatctgattccaacacctggagattacctggatatggagccagaattatcagagaggaggaagccaggctcagaggatttagatcctctgctggggccattagagaccatcaggttcaacccattcatttcataatcaaggagattaagagccttgcccgaggtgactcagtctgggcagagccaggctaaaagttaagtttccttggcttccaggccaggggggagacctcttattattattgttgttgcttatttgtttattttattttgttcttttgagtcggggtctcgctctgtcacccaggccggagtgcaatggcatcatcgtagctcactgcagcctcaacctcctgggctcaagcgatcctcccaccttagcctcccgagtagctaggactacaggcatgtgccaccacacctggctactttctaaaatgtttaattttttgtagagatgggggtcccactattttgcccaggctggtcttgaactcctgggctcaagtgatcctcccacctccacttcccaaagtgctgggatattaggtgtgagccaccatgcccagccaagatctgtttttagtgtatctggatctctgaccccagcctgaagagcaaggaggggaactactgggtccttgaacaggagttgaagggcccaggtgggataagctgatgaagaaccagagatgcggttgctaggaactccctcactgtctggcccacactgtcagcaatccctatgccaacacacaagcctccatgcctggaaccagcccaagccccagacctcttgcaccacgctctgaggacaatgggtcccagagaacagcaagttagctcctgactccatctgcagctccaggcaacatgaaccagctgttcatgggtcccaggaaaatgcaagctagtggttcagtggtgcttccagcaataccctcactacccccaaaggtgataagttgtgagcaaccggtagactggaacccacctgactcattcaagacatttggctccagtctccttcccttgcttacagtggggagaccctttccaaatggcatttatacgtctcagataaggttgtttgtcgtgggaggtaagggtaggggtggacgtggggagttcccagagtcatggggagggaagaactttccaatgcatcccccatctttcaacccaggcaccacctctacccctgttccattttgggggacatgaacaaaggttcccaggtccttaaaccttggaggaggcgagtaataaaacaggtcagcgactccaggccaagccaaagctgtgggctagcctgtaacccttgcttcacatggtgaccacttcctaccctccagtccagcaccctccagcctggctccgtggaactcttaactctcatcagatgtctaggcagaggcggaggcagctggtgagtccagggcactgtcagaaatgtagtccaggtatctcctggaccaagcgccctcctaaaagagggacagcatttcttggccgggcgtggtggctcatgcctataatcccaccactttgggaagccaagggaggcggatcatctgaggtcaggagttggagaccagcctgaccaacatggagaaaccctctctctactaaaaaaaaaaaaaaaaaaaaaaaatacaaaagtagccgggcgtgctggcgcattcctgtaatcccagctactcaggaggctgaggcaggagaatcgcttcaacccgggaggccgagattgtggtgagccgagatcacgctattgcactccagcctgggcaacaagagtgaaacgctgtcaaaaaaaaaaaaaaaaaaaaagaggaataacatttctctcttcatgtctatccccacatccttggggacattctttaaatctcataaatatgaatgatctctttggggagggggtgatcagagtcatagctgggcatgatcggagtcagagctgggaatgccggcagcccacccacccgcaccccactcccaccacaccccacacacctgaaatatcgatgcccttgaggccgatggcttcttgacactgggggagggaggagagggagggaagggagagcccagtcgccggagaggcggagacaaagaaggcagctgcacaaagcgcccaaagatgagagcgaaaaggacgggaatggaggcagagagcagacaaaagcggacaggagatgcggccgcaggggacaggcagggaagccggaggaggcttcgacaccgaatcacagacatgacacctcgtgactctgggcccagcagatgaaaaaaaaaaaatcctagcacgcaccccctcccctgggacactgtaaatagaaagcagcgtgacacacccccttcgccccggggtatggggggcggggatccgtggaggggaaggggtcaaggggaaaagaaagtcagaaaaaaggccgtaagagaacaaccttggaaacagagacggggagaggcgatgaagtgggggcgggagagaaggcctagggggtgggcagaggcaggggccgaggaagatggcccagccaaagggggtggggggagggccccagagggccacagtagtggagaagggggggctgtcctgcagggtcttcccgagtgagggtcttccctctgcctctaccccggattgggggcttgggcttgggggagagggagattgcaaagggagcgatctgcttggcgggggttcccctgggcgccggaccctggggacaggtgggagcgacatggcaacagaggggggcgggggagcgcttctggacacctgaagcgcagcctctgactcagttttctgggtcttcggactgggaggggctgggcagaagggggcgcagccgcttggtccccctgaggggcggtggccttctgctgccccattctgcggtggaggagcacgctagccccctccccagtccccaggcccggccgcccccctacctcgccggagccggagctggagccagggccgagatgccgcaaagccaagcggggcgggcggcgggcgcagacacccccggtgccaagagagaggtccctttggagcccccaagagtcaaaggaggatgacgcgcggggactcggccgcgctgtcggagcccgactctaacgtcggtcagcgctggggcagcgccgcagctccgttggcgacgcgggccggggcgcccatcggttcaggggttcgggtggcacgcgtggctcccccggcgacgctccggcggcagcttcgaggggcggagaccccggcgacgcggaagaaacccgggacggatccggagcggactcgtgccctgctggcggggccgggggtcggggtgcgccctgggcagctgggtggcctcaagccctactggcgagactccgccctgtgtcccggctggggagtgggactgaggatggtggtgtctcgggttttggtggtggtgatgctggtgttgctggcttagttatctccccaccccacgcccagcacctccgttctctgtccgggggtcccccactgccctgaagtacccctcctccaggagccacccctcaatggaatctcctctctccattataagagcggctgggaaggggactccgagccttcgtccccggaaactgatagagggaatcagaaaccctagaacaccgggaaggcgaagagacatcaagagacagaagttgaaaggctgaaaagggcagagggaggccaagatctctccatcttctcaccactgcgtgggtgcctgagcaccctggttccccgggtgcacaaggcccctaaatccacaatgtaccccacgggacagcaggcagaaagacctccaggtacacagcgcacacccaggcacattcacacagacctctgcactcaggaggcgctgcaccccacgctgatcctggtggctggggaggaagagggaagtgtggagctgggacccgggtgggtgcagcgctgcccctgctctaataaggcggtggcccaggctcttaactcctacaaggttctccatcccacctataaagtgagctcaagactgggtgcggtggctcaagcctgtgatcccagcactttgggaggccgaggcgggggaatcacttgaggtcaggagttcgagaccagcctggccaacatggtgaaaccccgtctctactaaaaatacaacaattagcctggcctggccgggcacggtggctcatgcctgtaatcccagcactctgggaggccgaggtgggaggatcacctgaggtcaggagttcaagaccagcatggccaacatggcgaaaccccgtttctactaaaaatacaaaaatcagccgggcacagtggcctgcacctgtaatctcagctacttgggaggctgaggcaggagaatcgcttgaacccgggaggcagagattgcaatgcgccgagatcgtgccactgcactccagcctgcgcggcagagcgagaatccaactcaaaataaaataaaataataaataaagtgagatcagaacaccttcagttctaccctgtaaggagccaggaaagtgatggacaaagccctttagcaactgtaaactgctttggagatgcagggaataattttttttttttttttttgagaggagtctcactctcgcctaggctggagtgcagtggcgcgatctcggctcactgcaagctctgcctcccgggttcatgccattctcctgcctcagcctccccagtaactgggactacagacgcccgccaccacccccggctaattttttgtatttttagtagagacggggtttcaccgtgttagccaggatggtctcgatctcctgacctcgtgatccgcccgcctcggcctcccaaagtgctgggattacaggcgtgggccaccgtgcccggccgatgcagggaataatgttaaggcctttcaaggtctgctggagtcctctctccaggggccgcccagctctcatgctccccctaccctctcccccaccccaaaattcattgttcacccatcttattccattttttctttctttactttccgaggtgataacttcataatcaggcaaactgatcccattggatgtgccaatgcaataagaaggaaggtcatgttcctggaatcccacatacaggaattccaaccgagggttaaagggctaaacaggaataacaccaagggttaaagggatgagaatcccgacttctaggctgaggcagggcaaaatgcaactgacaagttactcaacattttttaataggacttccccccatttctgatctctgaaagtgctaacacactgtgcgtctcaccaaactgcagattgcagttaaggtgccatatgcttcatgatgggaaagaacttcaaaaagttacaggtttcaaggaaaaaatcactagtggccaaagggagagaccatcatatgctaatcccgcccctttcgccagaagccacaccttccaccaacagaaactcttggattggcccaaccctcacacacttccagttccatcagccattcagaattaaaagtttaccaacgtttcggcccctccccttaagtccaagccactcagaagcaagttgtcactgtaagtcccgcctccaaacagctcactgggccaatggaaagccatcttgttccaggcttcattctcacacatctaggagagccggttttcctctcagctagagaacaagtggctttcagctgcctgagtgctcagaggttgcagggaggcggcctattctgggatggaggagagatctggctgagaggagacagccctggcgtggctggcctaggttccaggattaccctctacccagtagagtggtggtgatgtggggtgaaccactgaaccacccaatgtttactaagcactgtagcttaatctccataacgctgtaggggagggcggattgtttagccattttacagaagagcaaagtatattctgagaaggctatggaccagatcccataggaatttttttttttttttttttttttttgagacagtctctctctgtcgcccaggctggagtgcagtggcgcgatcttggcacactgcagcctccgcctcccgggttcaagcgattctccggcctcagcctcctgagtagctgcaccaccacgcccagctaaattttttttttttttttgagacggagtttcgctcttgttgcccaggctggagtgcggtcgcgtgatctcggctcaccgcaacctccgcctcccgggatcaagagattctcctgcctcagcctcccaagtagctgggattacaggcatgtgtcaccacgtccagctaatttttgtatttttagtagagacggagtttctccatgttggtcaggctggttttgaactcccaacctcaggtgatccacccgcctcggcctcccaaagtgctgggattacaggcatgaaccaccgcacccggccatgcccagctaatttttgtatttttagtagagacagggtttcaccatattggccaggctggtctcgaactcctgacctcaagtgatcctcctggctcaggcctgtaatgccaacactttgggaggccgaggcaggaggatctcttgaggccaggagtttgagaccagtctgggcaacatagtgagaccttgtctctaccaaaaataaaagaattagccgggcacagtggtgtgcacctgcggtcccagctactcaggaggctgaggtaggaggatcgctagagcccaggagtttgaagttgcaatgagttctgatggtgccactgtgctccagcctgggtgacagagcgagaccttgtctctctctacacacacacacacacacacacagacacacacgtatatatgtatatatatgtgtatctatgtgtgtgtatatatacacatacatacatatatacacacatatatatatgtatatatgtgtgtgtatatatatagagagagagagagaaagaataaagcgccaatcagtattagtgccattaataatggagataggccaggcgccgtggcttacgcctgtaatcccagcattttgagaggccgaggcgggtggatcacttgaggtcaggagatcaagaccagcctggccaacatagcaaaaccccttctctactaaaaatacaaaaattagccgggcatggtggcacgtgcctgtaatcccagctactctggaggctgaggcaagataatcacttgaacccaggatgtggaggttgcagtgagccgagatagtgccactgcactcccgcctgggcaacagagagagactcggtctcaaaaaaaacttttttttttttaagagacaggatcttgctctttcgcctagactggagtgcagtggcacaaatcatagctcactgcaacctcaaactcctgggctcaagagatctcccacctcagccccctgagtaactgagactacaggcacatgtcaccacacccagctaattattgtattttttgtagaggcagagtcttgctatgttgcccaggctgggcttgaactcctggtctcaagccatcctcctgccttggcctcccaaagtgctgggattacagacatgagccacctcgccaggccataataagccaccttgaatcttcatactcgccaagctgaaagcctgggcatctcaggattctggtgaccaagacttcctcctctggatttttgcttttgagtgtttccctcttagacccctcttctccctgtcccactgtgaaatcattgcgaagtcttctgccatcccccttatcacaccccattcggtgacctatctggacaattccccaatctatgaatcaagtctcgaccttccaggctgtctccaactatctgtggaacatctgtgttgtgatgagagcagtgaaataaaagatgtgaatgatttacaggaaatgacctggatccctcataagctttttctctttttattttattttttaaatttttcatagacacaccatcttgctacgttgcccaggctggtctcaaactcctggcttcaagtgatccgcccaccttggcctcccaaagtgctgggattacaggcgtgagccaccacctctggccaataccttgcatgctttacaacattcacaatcagctcctcttgcagacagaggaaggagaccagagatgaaagggttaagtaattggctccaagtcacagagccagtgagctcctaaaaggcttgctcagaatgatagactaaaaggaatcatctagatcatcccacaaggcagatatcagagatgccacactctcctggtgagtcatgaagtcacggctttctcagcctcaaagaggatgtagcacccaacttccagtaaggatgaaataaagtaatgtgtgtaaagtgcctggcagagcattcctggcacaacatttcaattgatggtaggtattattatccagccaactggagcagcaattctcacatggggtgtatcacagcctgtactttggaattcaggcataacagaagcctcttgtttattctgcagtcaggactgtcagggctcgaggaacacccacaggaacttcaggaatcaggcaagagaaagagggaaaatcactacgtgtgaccatgaactgttttatttgatttttggagacagggtcttgctcttttgcccaggctggaatgcagtggcacaaacacagctcactgcaacctcaaactcctgggctcaagtgatcctcctgtctcagcctcccaagtagttggcagcaccgatgcgcaccaccaagccctgctaatttttaaaaaagtttttgtagacacagagtctcaccatgttgcccagactggtcttgaactcctggggtcaagtgatcctcctgcctcggcctcccaaagtgctgggatttcaggtgtgagccaccgcacctggctataaactatttacttatttatttatttattgaacagagtttcgctcttgttgcccaggcacgatcttgactcattacaacctctgcctcctaagttcaagagattctcctgcctcaccctcccaagtagctgaaattataggcatacaccaccacacctggctaattttgtattttttagtagagaaggggtttcaccatgttggtcagtctggtctcaaactcctgacctcaggtcatccatccaccttgacctcccaaagtactgggattacaggtgtgagccaccacgccaggcttaatataaacctcttttagaacagggatgatgtcacagttatttttatgccactggtacttaggtacttagttcggttcttggcacatagtaggtgctcaattcgtatttctgaatgcacgaatgcctccagcctatgtctgcagttggggacagtgagacagacggatcacttatctcaggtcacccagttgacctgaggtaaagataaaacctgactcctggtatccctgttttctgtttgtttgtttgttttgtttttgtttttgttctgagacggaatctcactctgttgcctgggctgggctggagtgcagtggtgccatcttggctcactgcaacctttgccccccgggttcaggccatcctcctgcctcagctttccgagtagctgagatcacactcagctaatttttttgtatttttagtagagatggggtttcaccatgttggccgggctctcaagtgatctgcccgcgttggcttcccggtctgttttttttttttttttttttgagacagagtctccctctgtccctctgtcccccaggctggagtgccatggtggtgggatctcagttcatggcaacctctgcctcccgggtacaagtaattctcctgccttagcttcccaagtagctgggattacaggcacccacaaccacacccagctttttttttttttttttttttttttttaagtagagatggggtttcatcatgttgatctcacatgtttcatcacatttagtagagacagggtttcatcatgttgatcatcacctgatctcaagtgatccgcctatctgggcctcccaaaatgctggtattacaggcatgagccactgtgcaccctgattcctagtatcctaatcccaaaccaatccatagcccagcctcacactatacttctgaaggttagaagtgtgactagcgtgcttagatgtacatgaagtttcacatctgtgctcctccaattgtcagcttcctctttcccatgcacataaaccaatactggctacaaggtctgttcatttattcagcaagcattattgtcatcttctcagggcctggttaattccacttcttcttacaagtagctcactttagctggctgttaaaaatacactgttttgagccgggcgccgtggctcacgcctgtaatcccagcactttgggaggccgaggcgggcggatcatgaggtcaggagatcgagaccatcctggctaacacggtgaaaccccgtctctactaaaaatacaaaaaattagctgggcgtggtggcaggtgcctgtagtcccagctactcaggaggctgaggcaggagaatggcgtgaacccgggaggtggagcttgcagtgagccgaaattgcaccatgggactccagcctgggcgacagagcgagactcggtctaaaaaaaaaaaaaaaaaaaccactgttttatttatttatttgtttattttattttattttatttttgagatggagtttcactcttgttgcccaggctggagtgcaaatggcgctatcttggcccaccacaacctccgcctcccgggttcaagcaattctcctccctcagcctcctgagtagctgggactacaggcatgtgccaccatgcccagctaattttgaatttttagtagagacagggtttctccatgttgatcaggctggtctcgaactcctgacctcaggtgatccacctgcctcagcctcccaaagtgctgggattacaggtgtgagccaccatgcctggcctaggctagaccagcctgggcaacatagcaagactttgtctctacaaaaaaattttaaaagtagccaggtgtggtggtgcacacctgcagtcccagctactcaggaggatgatgtgggaggattgcttgagcccaacaagtcaaggctgcagtgagccctgatcgtgccactgcacactgcagcctgggtgacagagcgagatcctatctaaaaaaaaaaaaaaaaaaaaaaaaaaaagcccaacacttttttatctggagaatatttaacatacacaaaaccagagagaatacatatttcaaagtatcactttgtatatgataaatatatatagttttagccaggtgtggtggctcatgtctgtaatcccagcactttgggaggctgaggcgggtggatcactcgagactagcctggccaacatggtgaaactccatctctactaaaaatacaaaaattagctaggcgtggtggggcacacctgtaatcccagctacttgggaggctgaggcaggagaatagcttgaacctgggaggcggaggttgcagtgaaaatttacagaacaatctcatttcttccacaaataagttggaagaagagacagagagagacggagaggaaatctacagattaaaagaaaattaaggctgggcacggtggctcacacctgtaatcccagcactttgggaggctgaggcaggcagattgcttgagatcaggagttcgagaccagcctggccaacatggtgaaaacccatctctactgaaattacaaaaattagcccggctgggcgcggtggcttacgcctgtaatcccagcattttgggaggccaaggcgggtggatcacgagatcaggagttcaagattatcctggccaacatggtgaaaccccatctctactaaaaatacaaaaattagctgggtgtggtggtgtgcgcctgtagtctcagctactcggaaggctgaggccggagaatcgcttgcacccgggaggcggaggttgcggtgagccaagtttgggccattgcactccagcctgggcaacagagtgagactccatctcaaaaaaaaaaaaaaattagccgggcgtggtggcatacacctataatcccaggtactcgggaaattgagacaggagaattgcttggactcggcaggaggaggttgcagtgagccgagatcccaccattgcactccaacctgggtaacagagcgagattcagtgaaaagacagaaagaaaggaaaaaagaaaagagagaaagagaggaaggggaggggaagaagggaaggaaggaaaaaggaaggaagggaaagaaaaaaaaagaatcttgtggccaaatgtgtgggggttttttccccacacaccaagtaagcaatcaattctgcagccggtgtctttcaacccagttcaatctggacactacctggaggtagtgatagatcccacgtggaggctcagtcccacaagactgtcccccactaccaatgccactcacaagcctcaggttattctacctgcacgtctgactgaccggctatatactgggggtcccatgaccccctgttgggttcaattaatttgctagcacgactcacagaactcagagaatcactcacttatattcacctgtttattataaaggatattacaaaggttacagatgggccaggtgtggtgactcatgcctgtaatctcagcactttgggaggctgaggtgggaggactgcttgaggctaggagtttgagaccagcctgggcaacatagcaagactccatctctaaaaataaataaataaaatgaacaaacaaacaaacaaaaacaaaggatacggatcggccagaggcagtggctcaagtctgtaatcctagcactttgggaggccaaggaggaaggattacttgagctcaggagttcgagaccagcttgtgcaacatggtgaaaccctgtctgtacaaaaaataatacaaaaaattagccaggcatggtggtgcacgcctatagtccagtctacttgacaggttgaagcaggaggatcacttaggccggggaggcggaggttgcagtgagctgtgatggtgccactgcactccagcctgagagacagagtgacagcctgtctcaaaaacaaaaacaaacaaacaaaaacagggctgggctcatgcctgtaatcccagccctttgggagaccaaggcaggaagatcacttgaggccaggagttcaagaccagcctggccaacatggccaaaccccatctctattaaaaatacaaaaattaaccaggcatggtggcacatgcctgtaatcccagctacttgggaggctgaggcaggaggattgcttgaacctgggaggtggaggctgcatttcagcccgggtgacagagtgagaccctgtctcagaaaaaagaaaacaaaactatacagatgaagacatgcctagggtgaggcatggggagtggggtttggaactccatgtcctcccagagtgcaccaccctccaagaacctccacattctcacctatccagaagctctccaaacccagtcctcaggtcttttacaggggcttccttggataggcatgattgacaccagtgtagaagtgtgattggaagctggggcgtggtggctcacgcctgtaatcccagcactttgggaggccaaggtgggcggatcactagaggtcgggagttcgagaccagcctgaccaacatggagaaaccctgtctctactaaaaatacaaaattagccaggtgtggtggtgcatgcctgtaatcccagctactcaggaggctgaggcaggagaattgcttgaacctgggaggcagaggttgtggtgagccgagatcgtgccattgcactccagcctaggcaacaagagcaaaactctgtctcaaaaaaaaaaaaaaatgtgattggacaaaaagggtaccacctaatactaacacaccgagtggggaagcccagccaggcctgtctgttcagattctcctcggcctctctctgtgtagtgttccttcctccaggggatggggcaggaccctttctgaaatgagggtctcatgacccacaagcagacaaggtaggtcagagcatctcttaataaccagcttcagacagaacggcgagggaagaagattccgtatatttttagtttctatggcctgcctaggtgagaaaaaggagcaggagagaggaggccaggaggaggtcagagacagacagattgtgttttctgaagcctgcttctgaggccaaaagtgcccccacgttctaacaaaagactgtctttcacttttatcgctctgaagctgttccgaagttgcttcgggaattgaggacataggccaactactctaacaagagaagggccgattgttttagttacttaggaaataacaagggctttgggagttatgagccaagaacctcggacctatatataggatctataaatggatatatagataatagcacagtgccattaaaaaagagacctgagagagaccctcctctcttctccgtgtgaggttagggcaagaagatgacacacgtgatgaagtgaaccctctccagacgacatttcggctggtgctttgatctcggacgtcccagattccaaaactttgagagctatatatatatatttttgagacggagtttcgctcttgttgtccaggctggagtgcaatggcacaatctcggctcactgcaacttctgcctctcgggttcaagcgattctcctgcctcggcctcctgagtagctcggattacaggcatgcaccaccacgcccggctaattctgtatttttagtagagacagggttttgccatgttggtcagcctcttcactcttgttggctgggcacggtagctcactcttgtaatcccagcgctttgggaggccgaggtgggatgatcactccagcccaggagttcaagaccagcctgggcaacatggtagaaccactgcgctccagcctgggtgacaaagctagatcctgtgtcaaaaaaacaaaataacaaaacaaacaaaaaaacacagaaggtcgtgacagattttttttttttttgagatggagtctcactctgtcatccaggctggagtgcagtggcgcaatctctgctcactgcaaccttcgcctcccgggttcaagtggttctcggaccccagcctcccaagtagctgggatcaggggcacctgtcaccatgcccagctaatttttgtatttttagtaaagatgggttttcaccatgttggccaggctggagaaggtcgtgatagatctttaagcagagctatggggatggcgaggagggccctggaccagcctggcaggttggcaggttggggaaggtgtcttggaaatggaacctaaactgagtcttgaaactccccttccagataagaggtgctgggtccctcaggtgggaagggacagcctgagtaaagcccagggctgaaacacagcctggtttgcaagaacaagcagttctgggtcgtctgccctgggtggggctgcaccttggacccagcctgtaggcagaggctggctagcccccgaggacttcaagcaagagagggacagatttgccttctgtgtagagaccgccctctggtggccacatggaggatggcaggagagaatggcgccgaaagaaaagcggcaggctgagctcataaagctgcgaatgctgttccttcttcctctttttcaaaagacggattttgggccgggcttggtggctcacgcctgtaaccccagcactttgggaggccgaggcgagcggatcacgaggtcaggagatcgagaccatcctggcaaacacggtgaaaccccatctctactaaaaaaaaaatacaaaaaattagccaggagtggtggcagacgcctgtagtcccagctactcgggaggctgagccaggagaatggcgtgaacccgggaggcggagcttgcagtcagcggagatcgcgccactgcactccagcctgggcgacagagcgagactccatctcaaaaaacataaataaataaataaaataaaataaaataaaagacgggttttgctctgttgcccaggctggtgctgtggtgcaatcttggctcagcctgtaactcctggcctcaagcgatcctcccaccttggcttctcaaactctgggatgacagatgcatactaccataccccgcccatccccagtgtctggaaaatgtgcccactggagaaatctattggccggattgtgactggttggatgctcccacggcaggaaagggggaaggacaagtggccacgtcgaggcctgcctgaactccaagggtatccaggccgaggttgctcaaacccagagctgatttatttttatttttatttgtttttattttttgacacagtcttactctgtcgcccaggctggagtgcagcggtgcaatctcggctcactgcaacctccgcctccgggttcaagcgattctcctgcctcaatctctggagtagttgggattacagacacctgccactacgcccagctaatttttgtatttttggtagagacggggttcactatgttggccaggctggtcttgaactcctggtcttaggtgatcctcctgcctcggcctcctaaagtgctgggcttacaggcatgagccaccatgcctggcctgaacgaaacatttctatgctccaacttaaaatgaatgtagaacttattatctgagaatgacccagggaagtcatgggactgcccatattttcatgcaataaagggaaaattcttcccacaaataaattttataggacggtagaagcaaatatagggcattccaattacatttcaatgagttattcttgatgaagtcctggatacacaaagccagccagcaaataggattgaccaaagtcagaggatgctgtgaccaccccagggctctttttgtttgtctgtttgagacgggtctcgcttcgttacccaggctggagtgccgtggcacgatctcggctcactgcacccttgacctcctaggcttaagagagtctcccacctctacctcccaagtagctgggatgacaatcataagccaccacacctggctaatttctagaattttttttttcatttttttcatttttttttctttttagccggagtttcactcttgttgcccaggctggagtgcaatggtgcgatcttggctcaacacaacctccgcctcccgtgttcaagcaattctcctgcctcagcctcctgagtagctgggtctacaggagagcaccaccacgcccagctaatttttgtatttttagtagagacggggtttcaccatgttggccatgatggtcttgatctcttgacctcgtgatccacctgcctcagcctcccaaagtgctgggattataggtgtgagccacagcagccggccataggacataagcttttttttttttttttttttttgagacggagtctggctctgtctcccatgttggagtgcagtggcacaatctcggttcactgcaagctccgcctcacgggttcacgccattctcctgcctcaacctctcagtagctgggactacaggcgcccgccaccatgcctggttaagtttttgtatttttagtagagacggggtttcaccttgttagccaggatggtctcgatctcctgacctcgtgatccgcccgcctcagcctcccaaagtgctgggattacaggcgtgagccaccgcgcccagcctcagctgtgttcttaactctatttttccttcactggggatggtaccatttccccgtcctgttaagtgcccttgactgctcttacctttagctcttggaattccgccatctaccctaacttcacctccatctaaatcctgctcactcttcaaagcctgcctctccacgcatcctccctcctaactccatcccaatgcccaacagctctttgctaagcagatgcctttatttacagacaaagaagtaaacaggcaaaaggaagaggcttgtccaaggtcatgccacagactgaactctcactgggaggacagttcagggtcctgatacccaatctaaggcagcagctttgtagcacatcaggatgggggcaggtcatgcctggccatgttcacttccagaacgattatagctttaacaacatttttttttttttgagatggaatctcgctctgttgcccagactggagtgcagtggtgtgatctcggctcactgcaacctctgcctcctgggttcaagcgattctcctgcctcagcctccggagtagctgggattataggcatgagccaccatgcccggctaatttttgtagttttagtagagagggggttttgccattttggccaggctggtcttgaactcctgacctcagttgatccacctgcctcggcttcccaaagtgttgggattatgggcatgagccaccacgcccagcctactacagctttaacagtcttttttttttttttttttttttttttgagattgagtctcgctgtgttgcccaggctggagtactgtggtgtgatctcagctcactgcaacctccgcctcctgggtttaagcaagtctcctgcctcagcctcccaaatagctgggattacaggcacatgccaccatgcccggctaattaacaacattattttttaatttttgtagaaatgaagtctcactgtgttgcccaggctggtcttcaactcctgggctcaaacgatcctcccaccttggcctcccaaagtgctgggattacaggcatgaggcactgcgctgggcccagaactaccttttattggaggcatggagtccacagtgggtggaggtctcagtgccttgggctcaggggctcagcgaaaggccgcggccaggcctagatgaaggtgaggcaggtggaagcggggaccccacagctttccttgtgctcctcgaagagctgctccagggccgtcatgtagagggcgtgatagtgattgacttcctcctcggtggggtggaggcgctgggggacggggatggggcggcccactgcagggagagggagacaggtgggcgagggatccctgatgcccacgcagctgctggtggccgtccccccggaggtgggcactcacccacagtggtgatgggcacagcaaagggcagcaggccccaggaggtggctgagaagagaccgcgaccccagaagatgcaaggagagaagcccatgagcttcttgaaggtgagctggcaccaatgctgccaggagcctgtggcaaaagccttaagtctaaagatgtcattctccccaaaggagtacacgggcaccagggacgccctgggaaggagcaagagagaagagggggctcaggctgctggactgctccgagatgggcacccccaggggctacaagtccttcccactacgtccagatgctacctcccagggtcattcaaagcacatgactatggctacaaagaaagatctagaagacaggactcgcatttctttctctttctttttctttgttttagagagagggtcttgtaaccacacctgtaatcccagcattttgggaggccacggtgggaggatcatttgaggtcaggagttggagaccagcctggccaacatggtgaaacccccgtctctgcaaaaacataaaaattagccaggtgtggaagtgggtacctgtaattccagctactacagaggctgaggcacaagaatcgcttgaacctgggaggcagaggttgcagtgagctaagatcacaccactgcacttcagtctgggtgacagagtgagactttgtctcaaaaaataaataggctgggtgcggtggctcatgcctgtaatcccaataccttggaaggctgaggtgggtggatcacctgaggtcaggagttcgagaccaccctgaccaatatggtgaaaccccatctctactaaaaatacaaaaattagccagacgtggtgacgtgcacctgtagtcccaggtacttcagaggcttgaactcgggaggtggaggttgcagcgagccgagatcacaccactgcactccagcctgggtgacagagcaagactccatctccaaaaattaaaaattagaaaattttaaaaaataaataatttaaaaaattttaaaagagacaaggccttgctctcttgcccaggctggagggcagtggtgcaatcatagctcactgcagcctcaaccttctccgctcaagccatcctcccacctcagcctcccgaatagctgaaacttcgggcgtgcactaccacacttggctaatttattttattttatttttgtagagatgatgtctccctatgttgcccaggctggtcttgaactcctggcttcaagtgatcttcctgccttggcctcccaaagcactgggattgcaagtgtgaaccaccacgcccagccgggactcacatttcttgagacacagttgtgtagtgggccttctcatagtcattttctcattcggtctttgcaataacaacatcgtggcaaggtaggtatcagccctcttcgcaagtaaagaaacaagtccggccgggcacggtggctcacgcctataatcccagcactttggaaggccaaggtgggcggatcacttgaggccaggaattcgagaccagcctggccaaaatggtgaaaccccgtctctactaaaaatacaaaaattagcccggagtggtggtgggcgcctgtagtcccaactactcgggaggcggaggttgcagtgagccgagaccacgcctttgcactccagcctgggcgacagagcgagactctgtctcaacaacaataacaaaaaaagaaacaagtgcaaggtcttgcctttgagttggggtcagagagggaagccagggcttctgagaccgagggccctgctatttgcattatggtcctgtctccacggatgtgggagggagcttagggtaagcgggccccatgtttggtaccgagtcatgccctggagagcccacgtgggcctcggtgcagggcagggcgctggtctctgggcacccggatcccccatgcagggacctagatgtctggggactgagagagggcataaaccagcagagaactgacaggtagggcctgcacgcgcactcacccgtgcctcagcgccaggcgcacgaagcctttgcgcttctggagcgtaaggcagtgctccccggggactgaatacagggcctcgtgcgcaccccccaccatgatgaccacggcctgcccgagctggggctgggacaggatgaagtccaggctctggcggctcaccggacagagtcctgtgggcaggaggaggtggaaagtagttcccatctgcctccacggcacccccgcccctcacccagcctttagttccaagccccctacccccatctccaaggcggggaacctacccaaatgctcactgcaagtctgggcagtgctcaggctggcccctgtcctgggtgggggacttattatgggggggggtggtcccagaggggctggggaacatctggtcaggagtctcctcccgttctcctcctcccattcggctcaccaaaggacatgatgtagtcgcgatagaccgggaggtagaagaggccagccagcacggctaaccagggccggagccccgggaagagctgggagaagccattgctctcggtggagaaattacagaggaagcctgtacacatgatcccatgagggtgggcgcccagcacgtagttccgatccgggggcagctctgctgttttcaccagcttcggggtgttgagcaggatgaagggaggatggaaggcaagacaccggctgaaaagaggggccctccaccccaacagagttccgagttaaaggttcttaggatagggtcaagggccaccccagcagagttccgagttaaaggttcttaggatagggttaagggcccaggttttttttttttttttttttgagatggagtcttgctctgtagcccaggctgaagtgcagtggcacgatctcggctcactgcaacctccgcctcttggtcccggttcaagctatcctcctgcctcagcctcccgagtagctgggattacaggcatgcgaaactatgcccagctaatttttgtatttttagtagagacgcagtttcaccatattggccaggttggtcttgaactcctgacctcgtgatctgcctgccttggcctcccacagtgctgggattacaggcgtaagccacctcacccactctctctttttttcttttctttttttgaggcagagttttgctcctgttgcctaggctggagtgcaatggcatcatctcagctcaccacaacctccgcctccctggttcaagcaattctcctgcctcagcctccagagtagctgggattacaggcatgtgccaccatgtccggctaatttgtttggtatttttagtagagatggggtttctccatgttggtcaggctggtctcgaactcctgaccttaggtgatccacccacctcagcctcccaaagtgctgggattaaaggcttgagccaccacaaccgcctttttttttttttttttttagagacggtctcactctgttgctcaggctggagtacagtggcgcaatcatggctcactacagcctccaactcctgggctcaagcaatcctcccacctcggcctcccaaagtgcttggattacaggcatgagccatggtgccccaccaaggccccgctcttgaacatcatccactcccaggccgggcgcggtggctcaaggctgtaatcccagcactttgggaggccgaggtggctggatcacttgaggccaggagtttgagaccagcctggccaacatgttgaaaccctgtctctactaaactacaaaaattagccaggcatggtggcgggcacctataatcccagctactagggaggctgaggcatgagaatagcttgaacctaggaggcggaggttgcaatgagctgagattgcaccactgcactccagcctcggtgacaggagactcagtctaaaaaaaaaaaaaaatccattccctgtcccactgcctgagcttaggcagcggggagctcaggccccagcacaaggaagggctcctaggtgtgggagggagatggggagaggtaggaagcagtttttctgcagggtgacccatcaccttgacaggataataatcccttagttgtctccaaattgcccggttccttatccactccgaacgccttccacctgcggacaatgagatactggtggacgaacccccggagtcacctcccctcaccccccatgtccccaccatctctggctacctgggcccccatccggagctcaccttggttgggtgtgtcccagtccacatagagccacaccaagtaaaaaacagagaagggccagagtgacgtgaagaggaggacaaagacaagaagggagaagaaagggcctgggggaagggagagaaagaaaagagttcacttctgaaactccatcattccctccagctgctcccttcccttcctccccttcagaacaaccagctctctcctctggccctctcccagaccagtcacctggtcactgtcctcaggcatgcaagccagcagcccagccactctccctccaagccctactcccctggcagaccccaggcacccacgcctccccagctctcacccatgaagaggaaagtgagcacatattggtaggcgcccactgcttctagatgctgcttctgcaaggttttggaagtggttgggggctgcagggttgtggcaactcccattgcagaagctcccctcttccagcaggatcccagaacccggaggacaaacgatgaaggcttggatgtggacctgggcagactttctccctacaggagcccagctttgggggcctggctaagtcgcaaatcacctcccagagtagcgtgtgtccgtaggtgtgtgagtggggagatctttggatccagagccccagatgtctctgggttttttctcagcctgatctttgaacttcctgtcagtcaccagtctgggctggctgctgggtggggctgggaggcgtaaggggtgtgcaagggagaggtgacacccgagtgcctcaggaattgatgttggagtggacaagagtgatctggctagccccacccactgggtcccaggtgctggaaggggccagaagccctgctgagactatttgacctccagtcggggtgggggcagggcctggtccggggcatctccaggaatgtcaaggtcaccacccctgcctggacctcccccgccatgcacaaacacacacaggctgcaggtctgtttcccactttaatttttttctttttcttttcttttcttttttttctttttttttcacacgtctcggtcacccaggctggagagcagtggcacgatctcggctcactgcaacctccgcctcccgggttcaaacgattctcctgcctcagcctcccgagtagctaggattacaggcgcgccaccacgtctggctaattttttttttttttttttttggatttttaatagagatggggtttcaccatgatggccaggctggtctcgaactcctgacctcaagtgatccacccgcctcggcctcccaaagtgctggaatcacaggcgtgagccaccgtgccctgccgccagtctttaatttcacttccccacctgcagtcacccagcggacaggcgaggccaggcggtggggcgcgcctccgtgaggaccaggtgtctgtcggcggggacaccatagcgcgcctggtgctcctcgaagagctgcgtgagtcgccccacgtagcgcgcttgcagcgtgtccacctgggccggactgggcggggggctctgctgcacgggaatcgccgaccccactgcgggcgggacgggaggcgatctgggctgggatccccggttctcctcctcttttcctccttggggataggcgggaacagagttcagggctggcgcggtggccaaaggccagtctgggtggccccaacccgggacggggagggagcgggccctgggccaggatcgcggggcggaggccggggctcacctacggtgcggatgggcgcgcggaagggcagcgggaggccccggcggcccaggaacagctgcagggccacgcttagcagcggacgcagagcctcctgcgtcctctgcacccacgagctctgcgggttcgggaactgctggaagagctcattctccctggagaagacaggcactagggaggccgcaggagagcatggcgcggctgactcctggccccggggtccgcgggtggcgccactctcctcctccccggcccggcagccctggggcgtccccaccgggtcacgctcgggtcctcacccgagttccagagctgacttaaccaatctcttctgattccgaatcggcaagctcagttgtccgggttttgcctccagcgcctgcaggggccctcccacgccaggacagccacctggccaccactgaggcagcggggccttgacaaaggagaccaaacctggggggagggcagaagctgggggacccaacgcctgggtcttatttacttatttatttagggacggagtcttgctctgttgcccaggctggagtacagtggcctgatcttggttcactgcatccttgaccttcagggctcaagtgaccctcccacctcaagcctcccagtagctgggaccacaggtgtgtaccaccacacctggctaaatttttgtttttgttttttcttgtgtagagatggggtttcactgtgttgcccaggctggtcttgaactcctggccttaagcgatcctctcgcctgcgcctcccaagtgctgggattacaggtgtgaaccaccgcacccagccaaagcctgggacttggaaagaagagtggagaggacatggggaaaggagagctgggccccagtgtcccctgggaagaggatggtggggcgggagacagcagcaaggtcctcctcttctcaccacctgacatgatgtagtcctggaagaagaggagatggaaccaacaaggcagcatgagcaggtgtggcgggagtttggggaagaggcaggagcagcccgtgggctctgtgcagaagttggcgaaggctcccacgaccaggaccctgtgagggtggaagtcaaagaggtagttccaggaggtgcccaactttgcagttttaactagctggggagcagacgggcgcaccccaccccaacggtgagattgcactttgggagatggtggtcccaagagtctattctgaagccccaaccgcacccagcaccccacagcccaggtcctcactgagagggggaagtagtcacagaaatgtctcaaactgcccagttgcaaacccaggcagatctgcgggctccagccctgggtatgtttctatcccaatagagccagaccaggtagaggaccgccagcatccaagcccagccgaggaatgcaagagccaggctgccaggtgcacctgggctggaagatggggcagggggagctcacagtacaggtgtttaagaccattcagttgttgctcctacccatccagtggcctgagtggtgggagctgcagaccagtcttctgtggcaggctgaatgctccagtcttcagtagggaactgctagatagcacagagggcacctgcacgccttcagaccagtctgcaacctcaggttgagtagcagtgaactcaggagccggagcagtccattcaccctgaaattcctccttgtcacagccttttcagcagcagcctgctcttctttttcaatgtcttcatgatctctgtagaagtagagatcaggcatgacctcccacgggtgttcacaggaaatggtgccacgcatgcgcagaacttcccgagccagcatccaccacatcagacccactgagtgagctcccttgttgttgcttgggatggcaatgtccacatggcgcagaggagaatctgtgagacacagagcaaggtaggtaggttaacataagatgcctctgtgagaggctgctgatcagccctggggtcagtaaacacaagaagccgtggctcccggaaggctgcctggatctggttagtgaaggttccaggagtgaagcggccagcgattggagtggctccagtggcagcagcaaacttcagcacggccctctggccagtatttctggagggtatgacactgacatcagcggggttttcaatggcaacaatgccacgagctgccagcagaagcttctcccaggtcctcttcagatttgggatgtagatgccatcacttttccttttatacatgtactgttccatctgtaagtcaaggttggtgccacctaattggtttcctgctgcaaggaaattaaggacatcttcctccttcatttgcaggacatcaagggctccggatgttgtgaaagtttctctttaagttacaacaggaatccagaacaacgccggatggagccctctgtgggtagcgtggaaagctttttttgttttgtttttttgttttgagacgaagtcttgctctgtcacccaggctggagtaccgtggcacgatcttggctcactgcagcctccgtctcctaggttcaagcaattctcctgcctcagcctcctgagtagctgggattacaggcctgtgccaccacacctggctaatttttgtatttttagtagagacagggtttcaccatgttggccaggctggtctcaaactcctgacctcaagtgatctgcctgcctgcctcggcctcccaaagtgctaggattacaggcgtgagccactgagcccggcttatttatttttttgagttggagtttcattcttgctgcccaggctggagtgtgatggcgcgatctcagctcactgcaacctccccctcccaggttcaagcaattatcctgcctcagcctcccaagtagccgggattacaggcgcccgccaccatgcccagctaatttttgtattttttagtagagatgcggtttcaccattttggcctggctggtcttgaactcctcacctcgtgatctgtccgcctcagcctcccaaagtgctgggattacaggcatgggccaccgtgcccagcttaattatgtattttttaagtagagacagggtttcaccatgttggccagggtggtctcaaactcccaacttcaggtgatccatccacctcagcctcccaaagtgctggaattacaggcgtgagccatcgcgcccagctaatttattttttgtagagatggggggggtctcgctatgttgtccaggctggtcttgaactcctggcctccagtgatcctccgcctgccttggcctctgaaagtactgggattataggtgtgagccactgtgcccagcctgggaagtccctcttttggtctgtgccctctcatgcactttttttttttttgagatggagtctcactctgtggcccaggctggagtgccggggcacaatctcagctcactgcaagctctgcctcccgggttcatgtcattctcctgcctcagcctcctgattagctgggactacagatgcccgccaccacgcctggataattttttgtatttttagtagagacggggtttcaccgtgttagccaggatggtctggatctcctgacctcgtgatccgccccccttggcctcccaaagtgttgggattacaggcgtgagccaccgcgcccggcccctctcacacactgtctccagctcaggcaatacaggggaaggggaaagtgggagccagggaagctcccaggttcccacgagggtctaacattctgcctctcccttttcctcttgtcttcgcttttctgggtctctcccctctacgctgctggcaggaggggagatggggttaatgggcagaccaatggactcaagacttacaaactgtcctttattcagagtgagactgcggaacattaataatttatcacgcggggagtccccagaagccctgtgcccacgaacccctgtgggcggaggagagaggcggggactccgggagcttcctgagagggccgtgtcttgggagcaaggtgacatattcagttcaggcacgcggaacatgaactcaggaagtggggagacagagagacccatcccccaactcccaggacgggggccaggccccctaaaaaggcacaatggcagggcaggtttggcagagtggttgagtgtcaggggtcgacaaaggacaccatgatgtagcgtgtgccccaggtcgttggcagcccctcgtggtagtgggtgaggcggccggggtgcaggagtgcccagcccttcctcggggaggagatcacacagtcgtagcgcaggaagcggcagccacctccctggaaagagaagggaaaggaaacatggagtgagcagacgtggggcctggggagcaggcagatacacggggcagggcggccagaccggggggcacggtgcagcagaccggggtgacaagggagatggcagatatggcagacaccggaggctggggtgcctgcagacaggaagatccaggaggaagcagaggccgcctgcccagaagcgatccaggagctgatacccacaaagtcactgggaaatgggcagggaggggagctgggggatgcacgcaggggctctaggtggggacccgagggtcctgaggtgaagcgtgggtgggtagtgtgactggggcgcacctcatagtccaggcccttgtggttgagggcaacgttgagggtgaaggtggatgagtcgtggtgtggccgcagagacggctgctcgtctggccggtagcgaaccacaaagttcatcaccgcccgcgcctgggggagaggagggaagaggctgcagggacagctgcgggcgcagggggtcttttattttgtattattattattcttttgagatagtctcgctctgtcactcaggctggagtgcagtggtgcgatcttggctcgatcttggctcactgcaacctctgcctcccggtttcaagcaattctcatgcctcagcctcccgagtagctgggattataggcacctgccaccacacccggctaatttttgtatttttagtagagacgaggtttcaccatcttggccaggctggtcttgaactcctgacctcaggtgatccacccacctcagcctcgcaaagtgctgggattacatgtgtgagccaccacgcccggccatcgcagggtgtcttcagggagatgacggtccctgtccagggaggggaggggaggggcggcgggacccctgcgtaggaggctctcgctgctcactggcattaaattagggccaagcctcaccttgggtctcagcctggcgccctccctcccctggggtgcctcctgcagcgtctcattcccctgggaactaaaatgcatggagcccaaatgctgccggggccgaaaggggtctgcgtggagacttcctgtccgggactggggtggggaaggtggggaggcagctggcaggtgggcagcgcaccttggtgtggtaaccgggaaacaggctctcggtcatggggcccacatacgtccgcagcagctgcagccactggtcctcgtaccccacctgcttcatgtggatgtccacggtgggcacattctcgtagcctccagccagccttgaatcctgggggcggcgggcactgagccacccgcccctccagccatggcgctccccaccccagggcagtccagcctccttcctcccgtcctccagccttcacacctgtgttcctctgtgcagaacagtcttctttgctacttctcggcctgactcactgctcccggcccttcaagtctcagctaagatgtcacctgtcctgtgaagctccctactcctcccaagtctgggtcaaggcttccccgtggcttctattgcacccaggctgctcatcgcagccttagaagctgtgtggtggctgctgctcacagcagggcagagctcgaccggcatgaggcattcctgcagtgcagtctaggacagtggcccaggaatgatttccttttctctttttttagagacagagtcttgctctgtcacccaggctggagtactgcggcatgatcatagctcactgcagcctctaactctggggctcaagtgatccttctgcctcagcctcctgaatagctgggacatgccaccatgcccggctaatttgttggtttttctgagatggagtttcactcttgttgcccagcctggagttcaatcttggctcactgcaacctctgcctcccgggttcaagcaattctacctcagctcccgagtagctgggactacaggcgcccgccaccatgcccggctaattttgtgtttttagtagagatggggtttctccatgttggtcaggctggtctcgaactcctgaccccaggtgatccacctgcctcggcctcccaaagtgctgggattacaggcgtgagccaccgcacctggcccaaactcggctaatttttaaattttttgtagagacagggtctcgctatgttgcccaggctggcctcaaactccccctgctcgagcaatcctcccgcctcagcctcccaaagtgctaggattacaggcatgagccactgtgcccagcagtaaatatttcttgaatacatgaatgaatgcagttgtgaccccagggtctgcctcccccctgggaacccctctttgctcccattttcctgattccattgtttgtttaccagatcctgcacctcctcctctgggaaggcctctgccctcctcgcccaggcccttacctcatgccggccgcctgaccactggccgtagtgctccatctctgccaccagctcatcacacatttgttctgacagcagtgggaaccagtacacgtccgggcatggctgaggatggggcgagggagaacagggctagctgacgccgcagaacggggaaggggacaggcagcaggcagaatgggaaggggcagggaggagcagggaaggctttgtgagagcagccggggacccgccaggtaggggctgagggagtggcaaggctgaggaccggtttctggcacaggagaggtggcagagggtcctggagcccctcgtgctgggacagtggtttccaaacttccgtaacagtgcatacctcttttttttttttttgagatagggtattgctctgtcaaccaggctggagtgcagtggcacaatcatagctcactacagcctcgaccttctgggctcaagcgatcctcccgcctcagactcccaagtagctggaactacaggcacatgccaccatgccaagctaattttttcttttcttttttttttgagacagggtctcgctgtgtcacccaaggtggagtgcagtggcgtgatcttggctcactgcaacctctgcctcccaggttcaagcgattctcccacctcagcctcccgagtagctgggactacaggtgcacgccatcacacctgactaatttttgtgttttttttttttttttttttggtagagacaggatttgccatgttggccaggctggtctcgaactcctgacctcaggtgatctgcctgcctcggcctctcaatgtactgggattacaagcatgagcaccactcctggcctcatttcttatttaatttttttgttgttgttgtagagacagattctcgctatgttgcccaggctggtcttggacttctggcctcaaccgatccacccaccttggcctcccaaagtgctgggattacaggcatgagccactgagcccagccccattcttcttttaagatgtgatttcaagcataacaccagtatacccaatatacaggagaagaggctgtctgatcaatgctggctttgggggcaagagtcctttctgttcggcctctgccttcatcttgtctgaacagaaaaccctcagatctctggggaactcagtgtgaaaactttgaatccaggcgggggcccccaagaggcgatggccatgagggcagggggtgggtggggaggctgcgtgggctcacctgctccacgattccttccccttccagggcccggctgtagttctcgtggatgtactgctccttccagtcctgtgagagggtggggggcacatcagatctgtgcccccctcgctcccagcccccaggggaccgccccctcccactcagtgtcctgcgctggcagcagccatcacgtttggtgtcttccttagcacaaggcggggaacctgtgtgctctgggcgtggggtccccactcacgacggggttgtcgaagatctgccagaggtcggggtgcaggtgctccgtgtcgtatctggaagtggccaggagccggccaaattcatgctgattgctcagatggaggaagatgccctgtagtggggtgggggtgtccgtgatgcaggcgatgcctggagtctgggggactgcccccaggccagaccgtgcacccgcgctcaccttgtctcgaaagctcttacagaaggccatgtccgggtctgtgtcactgcccgagaacacatccctctggggcagctccatccgcagggtatcaccccggatcacataggcctgggagatgtatggtacattccacacacccctggaggcaccgacaccgcggtcagctgggaggacagccccccaaattctgcccagctcatcccgggaagtccttcttccctcagggtatcccagtccctgaacataaggcccctgcatgtcccttgtcaagcactgctggtcccaaccaccccacctcccaaaatgcggtcagttcctaccccaagctgtggcacttcttttttgtttcttgtttttggtttttgagacggagtcttgctctgtcacccaggctggaatgcagtggtgtgatcttggctcactgcaacctccacctcccaggttcaagcaattctcccaccttagcctcccaaatagctgggattagaggtgccagccaccatgcccggctgatttttgtatttttggtagagatggggtttcaccatgttggccaggctggtctcgaactcctgacctcgagtaatccacctgcctctgccttccaaagtgctgggattacaggcgtcagccaccgcggccggcccttgttttttattttcttgagaaagggtctccctctgtttccaggctggagtgcagtggtggaaacatggctcactgcagcctcaacctcccaagttcaagtgattctcccacctcagcctccgtagtagctgggaccacaggcacgcaccaccgtgcccagctaatattttaagttttttgtagagataggctcttgctacgttgctcaggctgatcttaaactcccaggctcaagcagtcctcctacctcagcctcccaaagtggtggcatcaaaagtgtgagccactgcagccagcctcatggcacctctgactggtgaccactccaaacccctgagagtagggggcttgggtctacctgggccccgggtcggaggaggcggggggctgcaggctggcgggactcacactcgcttccgctgcaccagctccacgtagtcctcggagcgggcgtagtactcatcggggctcagggcgccccagaagttggaccacagcttgccgtggcgggacagcatgggggcgatcaccttcctgcggacaggtgggggtgagggctgggcagacgggagcaggcctggggagcccggtgcccagggcaggagtggggttcccggggcctgttggggtgccctccggctgcggggagagggggtaagccacctgttctcctcaatgaggatacgcagggtctgcaggttggtgaggacagcgtcggcgtccaggctgaagtagaactcacactcggggtcctgccgacacaggtccctggaggtgagaggcgagctgagacggcggcaggtggggaggcggtgtggatggggtaactggcccatgcccccaggagaagaggagggaggcagtgtctatccttccttccccaggctctctgctgtctgggtgagctggcaagggcaggagtgacagcatgggggctgcaaggatgcgaatggggaggcccaggaggggctggatgggtgacaggtgaggcaagggccaggcagcaggtggcagcacccctgaccctacagcctcatcccaccctctcctccccgcaagcacccgctcacatggccatgtccctggcctcgcctgggctcagagcctcctccggccccacgagcttcacagctgagaagtggtcctggagctgcggccaggagtcagcgatgtggggttcatggaagacctcctgggaggggaagacatagggggatgggctcagagggcagggaggcggcacctgagggatgggggtgcaggaaggagatgggggtgggggcacgagagttctgatcagggcagggaaagggtccctcaggagaggctccaacaggggagtctcacgttgttgtgcaggaaaagggtgaccctgtcggggggatagtccaggagtagcagccgctgcaggaagcggggcagaaacggagtaggctgttccacaaacacggccagaaacacccgggggggaggctggaagatgcaacacgcagggactcagagagaagcccggacttccctgctgcccccacccaacctccaccctgacaggtgcagggacccaggagcgatgcccccaccgccccccagtccgctgtccttgtacctcctgctcagcacgctgccctctcgtgcccctccctggcacccccacctcacctgcccccccgggagtgtcctccggtcctggttgcagaagccacagcctccctcaggagtccagccattggggacgtagtttcccaggtagttgagctgcagctgttggggacagactgaggctgagtggctgaggcctccaggggtggaggtgggggtcaggcacctctgatatgggggctgggaagggccagcttctcagaagggttggaggtgcctgggggttggggcagggcggggcgggggttgagaccactggtggcaccttagtgggaccgtttccatggaccacaatggggagcgtgtcgtaggccacgttccggatacgcacacggttccgatcaaactttaaaaccacttcatctggggaagaaaaaggccaggcttcagaccccctttctctgggagctaccaagcaacacattcttctaaatatggggtcccaaattctccagggaataaagggacaccagggctatcctggagtgtggctcgttgtgggcaggaacctttgggttctgaccaggctgctggcccagagagctccactgcctactacctgcgtgatcacaccgcagctgggcgaggaaggggagggtgagctgagcgggaggccagtaggagcctcagcatccctacccctcctcatattctctctctccttgtttttttttttttttatttttattttttgagatggagtctcactctgtcacccaggctggagtgcaatggcgtggtctcggctcactgcaacctctgcctcctgagttcaaaagattctcttgcctcagcctttcaggtagctgagactatgggcacatgccactacacctggctaatttttgtatttgtattttttgttttttatgctgagatggagtctcactcccaacacgcaggctggagtgcagtggcacgatcttggctcattgcaacttccacctccagggttcaagcgattctccttcctcagcctcccgggtagccaggattacaggcgcgcaccaccatgcccggctaatttttgtgtttttagtagagacagggtttcaccatgtcgaccaggctggccttgaactcctgacctcaggtgatccacctgcctcggcctcccaaagtgctaggattacaagcatgagccaccacacccagcctaatttttgtatatttagtagagactgggtttcactatgtcagccaggctggtctcaaacttctgacttcatgatctgcccgcctcagccttccaaagtgctgggattacagatgtgagctgtcgcacctggccttttttttttttgagaaggaatctcactctgtcacccaggcaggagtgcagtggcgcaatctcggctcactgcaagctctgcctcccgggttccagtgattctcctgcctcagccttctgaatagctgggattacaggcacccgccaccacgcccagctcatttttgtacttttttttagtagagacgggttttcgccatgttggccaggctggtctcaaactcctgacctcaagtgatcctcctgcctgggcctcccaaaatgttgggattacaggcgtgaggcaccgtgcctggcctctctctctccccgttctccccatcccctgaggcaagtcaccaacccctccacaagctgttttcctcatctgtacaagaaggataaccagggaccttccctttgttaaatggaatcacagaagtagaccgggcacagtggctcatgcctgtaatcccagcactttgggaggttgaggcaggcggatcacctgaggttgggagttcgagactagcctggccaccatggtgaaaccctgtctctactaaaaatacaaaatgagctgggcgtggtggcgcatgcctgtaatcccagctactcgggaggctgaggcagaagaatcacttgaacccgggaggtggaggctgcagtgagccaagattggaccactgcactccggcccgggcaacagagcgaaactctgtctcaaaaaaaaaagtccctggcccggtgcccaggcacagcatgtgtgcaacagatgccagacacgtttttgcttcccttgcttctggtagtaccatcagcacagtgcaacttacacaaccagccactcagacgcctgacaaaaccacatagaggtgggcttggatcagcccagctcctccagaagcctctcccagctcaagacccctagctgcagaggctgcgcatcgcccacctgcggctgtcttcctcctcacctaaagccccgttgaggttctgaaagatccgagacttatgatccagattaaggctgagtttctcctaaatggaatgagatgcgatggagtggttaaaatggaaggagtggaaaggacatcatttctcacttttctctctctttttttcttctcttgcttttgttgcttaggctggagtgcaatggcacgatctcagctcactgcaacctctgcctcccggtttaattgattctcttgcttcagccttccaagtaactgggactacaggagagtgtcaccacgcctggctaatttttgtatttttagtagagacggggtttcaccatgttggccaggctggtctcaaactcccaatctcaggtgatccacccgcctcagcctcccaaagtgctgggattataggcatgagccaccatggttttcctttttttgtgagagacagagtctcattctgtcacccaggctggaatgcagtggtgcaaaacatcctcaaactcctgggctcaagtgatggtcccacctcagcctcccaagtagctgggattacaggcgtacaccatgaagcctggctaattttttctttctttttttttgtagagatgaggtctcaactatgttgtccaggctggtcttaaattcctggactcaagcaatcctcctgcctaagcctcccaaaagtcttggatgacaggcgtgagccaccacgcccaggcacacatttttctctttgcaaccccctttgctccccaaatcccattcagcctgaggcctccactcccacagagctgcgggatgcgcccactcctctgccttccctaccctcagtcctgggtccaggtagagccgtgtgtagaacagctggtcgtcgtcatcatccttgtacttccactggcgcacgatttggtggatggtggtggcaaaaccgatgaatcctggcggggagggggagtgttgacctcgagactcccagggtcccagggcctgtcccccaggctgtctccagccctcaggcgcctttaacactctgtgtcccaccgctcttggcccctctgccttgggcactctgccactcaccaccagaattgaggaagcgcttccccgtgcccacctcagggtactgctccgccagcccccactcgggccagcagaagctctctgcagagaagagcaggcggctgccactctggacgaacttcttcagcagctctgtggggctgccggccagaatcacgtcgtagctgggtgaggaaggggaggatgggcaggggtccactgggaacctcagcatccttaccccgctccctatccccagccccgttacctatccacaaacatgatgatcatatcctcccggtcagcgtatttctccatttccttctttaaccaccggaccttctgtcctccaccaactgttcgagccacatcaccccctcgccactcctctcccaggcccagggtctgtggagaagattgccccgtgccaggcaaggggtggggagttgtggggggaactcctgaccaggcttaccgtggggacctgggcatccagacttcagcccacccctcctgctgggaccccctcccaggggcctttccctctcctcacccgcacagtgtagttgaagaactccgcagagcgcaggaaacgcaggtacccctcggtttcagctgtggccacagtgatcaccagcagcttctctgttaccagagggaaatggcacttgagatccaccgcccagctccgggcctcacgtgctttgctgtcctccctctcactcgacttccctccctttcttccttcccattcttaaactcttctcacccaggaacactctgaccccaaggagggcagggtgggagtggtgcccaggaattccgagtccctttgcatagggaggctggaaaccccagttcagggtagtgatgggcgaggggcttggcgcgactcagcctcaagagcacgctgggcgggggacgggccagacacctcctcgagcctccgaccctctccgggccaggctggcaggcacgccagcctctgccccctcggccgtgccgggcctccccgggatctcacctgggttgaccgggtctcggccccggggccggtcggaggctgaggccgcagggggcagcagcagcggcagcagcagcaggaaccggggtccaggccccgaggaggtcatggtggggagcgggcccagacagcacccaggatcctgggatctccgctacgcgcctggatcccagctccggaggggagctctggaaagggggcgctcgggctgctgtgcggccgccgcggggagcagcttggctggggctacgccctgaaaaaaaggcgtatccggaggctacagaaagctccagatgccggcgccacagacaaggcgaggattgtcccgggcgcacgggaggcgggggaggggcggcggctcgggccggcgggcgggagacagggactctgggctgagccagccgcttgacacatgtggaagctgaaacctggacaggggaagtgacccctggcggagggtcgggcgggacgcgggggcgggtcctggccgcagccctgcccagagagtgcgggcggaagacggggagagcgagaggctggcggcgacagggagggaggaagcctaggagtccgccgcgggacggaggcctgggggaactgggagttcagctttctgcagagggccactaggaacctcggattgcccacggaagccagccactttctttgacagtccagcccacctcctcttctgcccggagaagctccaggggctgcctttgtgatcacagcatcttcacaaggaccaaaggaaaataagatttcttgtaagaacaccgtgaccacatctttaaaatgacccatttcgtggctcccacaagatttacacctccacactgaggccggaagtggttttgcccctataaaacatggcgaaaagctttcttgtctccaaggaaacgccacgtaatgagtcaaagctgtggcgcacgcgcagaagtacaagctaccggaagtgatggcgcccctactaaagccttggggttagtacgcgtgcgcagcagtttcttccgacagttgtgttgtgccaatggtggagaagaaaacttcgggtatgtgagcccccgcggttcgcccccttccccttcccaagtcagtcctagaattttcttacctagtcattcctcttttttggtctcgctctttcgcctaggctggagtgcagtggcatagtcatagctcactgcagcctcgatttcctgggatcaaaccatcctcccacctcagcctccggagtggctggaactgcaagcatgaggccccacgcctggttaatttttaatttgttatgtggtgacgggctctcgctatgttgcccaggttggtctccaactcttggcctcaaagcgatcctcctgcgtgggccttccaaagtgctgggattacaggcgtgagctgccgcgcccggccccagctattcctcgtaaatcccttcctcagccctcgggtcctggcgttgacccttcattcacgttttattggtgcctgctccaagccgggcgctggggatgaagaggagaaagacccctcttatccaagcattggctttttctggaaggggggcagaaacgcagacctaacactgtattttactgtgatttgtgatttgtgctatggcagagggaagcacagcgcctgggaacagggagacgtggcatctagcccaggttctgggaggcaggggactgtgaaagcttgaaggaaaaatgaaaaagcttgaattgagtcttgaaagtctagttagggtttctctcagtgggaacctcacgtgcaaaggcagtgaagcaagaaaaagcaagtcgcttaggaagccaagttcacagtttggatcttacagagtaataagaaaggatcgaaggctggggaaaggttacattttgaaaggcattatacacactgcccaggagtttgaactctgtagggaaacactggagagccagcaaagactttttttttttagacagagtcttgctctgtcacccaggctggaatgcagtggttctatataactcactgcagcctcaacctcccatgctcaagtgattcttccacctcagccacctgagtaggtggaactacaggtgggtgcaggccaccatgctgggctagtttttatattttttgttgagatgggtgtctcactatgttgcccaggctggtcttgaactcctgagctcatgtgatcctcctgcctcggccttccaaaatgctaggattacaagtatgagccactgcgcttggcccttttagtttttctttctttctttttttttctttttgagacggagtcttgctctgtcccccaggctggagtgcagtggcgcaatctcggttcactgcaagctctgcctcccgggttcacaccattctctggcctcagcttctggagtagctgggactacaggcgcccgccaccacacccagcttatcttttgtatttttagtagagacagggtttcaccatgttagccaggatggtcttgatctcctgacctcgtgatccacccgcctcggccccccaaaatgctagaattacaggcatgagccactgcgcttggccctctcagtttttcaaaggcatcgcagtctcccagccatcagttttttcatgttggcctcaaggtctgaaataccccgctgtctcccctagccaggtcattcaccagttaggccttaacctagacttaccttcttctgggaagtcttccctgactactccaaggccaggttgggtgtttctgtcctgggctctgcagcactttatacttccgtatcctagcctactccatgctgtacttactgtgtttgtttcattaatcatttgtgtttgccactggactgtaagctttggcatccctaaaacctagagcactgcctggcatttggaggggagacagagtagtacagtgatcataatcatatttttttttgtttggttggttttgggtttttttttttttttttttttttttgaaacagggtcttgctctgtcacccaggctggagtgcagtggcacaaacacagctcactgcagccttgagctcctgggctcaagcaatccttgcgcctcagcctcctgaatagctgggactacaggtgtgtcccactacacctggctaatttttgtattttttttgtagaaacatggttttgccatgttgcccaggctggcctcgaactcccgcgctcaaacaatccacccaccttggcctcccaaagtgctgagataacaggcatgagccactgcgcctggcaaggtcatgctttttgatgctgggtagttctttgactcctgacctggccacttgttagctatgtgaactttggacacatttcttaatctctgaatgttggtaaattggggatgatgatactgtcccatacagctgttgtaagggttaaatggttcaagtgttgcaaagagctgagcacgtgcctggctcatagtaagtgtccaacaagtgatagctactggggctgctgctgttattattatgcttaacaggtggttaatgaataaagagatatattatgcaaaattatcagaatctggcaaacagctgataaggcaatgaggaaagaggttttttgttttttgtttttgagacagggtctctctctgttgcccaggctggaatgcaatggcgcgatctcagctcgctgcaacctccacctcccgggttcaagcgattcttgtgcctcagcctcccgcgtagctgggattacaggcatgtgccaccacaccctgctaatttttgtatttttagtagagacatggtttcaccacgttggtcaggctggtctcgaactcctgacctgaagtgatgagcccacctcagcctcccaaagtgctgggattgcaggggtgagccactatgtccagccgaggaaagaggtttaaaataacttctaggtttctggtttggatgtctgcatagacggttccatttgcgtagagagaatctgcaggagaagcagacttggatgggaatttttttttttttttgagttgcagtgtccacagggccagtgggtgatttcttttttccttctttctttttttttttttttggagacagtcttgctttgtcactcaggctggagtggtggcgcgatcacggcttgctgcacactgcagcctcaacctcccgggcccatgtagtcctcccaccgcagcctcccaagtaactgggactacaggcatgtgcaaccacgcctggcttattttttaattttttgtagagatggggtttggctatgctgcccaggctggtttctaactcctggcatcaagcgatcctttggcctcccaaagtgctgagattacaggcgtgcgccaccacacccggcctcaccaggcgctggctgcatggccagttctggagcccgggagcaggttctgtgttgagggagtcactctgggagtcatttgcttgtgggtgatcatcagagccctttgtttggagtcaaaatcccaggaagagtgcatagactgagcagagaagagggccagccacctccatagaggcagggagagcagccgagctggagccggaggattcctggacatggagaaggcctgcagttttgtcaaggaagcaagcaaagggtctctttctactcgatgaaggtagtcggcaccgccacagggtggacagggctcaggtccaaggaggaggaggaaggcacactggagccgctctggggcccagagcgtgggtgggtggtcccccaggatgaccaggcaggtgtcagattttggggttcaaggagaggatgggaggtgaaaaagtgacacatcacgtgagttgctgtgaaggagaaggagctgtgaagccgagtagacacatgagcgtcgccagctctttgatggaaagtgccggttgaggaggaggggttgaagatgggagagggagggaatgggctggaggtgaaggctcggggtcagtggtgggacttggggcgctccagggcaggggtgtgcccacttggcatcacgtttgtgggtaggtggcctggaagatgagcgtatccaagcatgggtggcccctggttcctttgaggtaggaggtgagatgctgagtgtgagggccatgtgtattgagggcccaggacagcctggaagccccagggcgctgtgacctccccagtcagcagtgagctgtctccaggccggggctcactggctgggactgggattgagaggaaggcggagaaaacaggcaggagaaactgaaagggtctcagctggcgggacaggaagtgtgggcccagggcttggcccgggaggagacaaggggaccaagggcttggcaatggggatgagatcagagagctgcagcttccaccccgggtgccttcctcgccctgcccagagcggcggccacactgctctttcttggaagccctgtaactttgcgtgccctgctccatccagttcgctcccaggaccccgggcagcggcgggtgctggaccgggctgcccggcagcgtcgcatcaaccggcagctggaggccctggagaatgacaacttccaggatgacccccacgcgggactccctcagctcggcaagagactgcctcagtttgatgacgatgcggacactggtgaggcggatggaggaggaagcgggggatgggactgagaggagggcgggagagtccgcccaactcaggctgtgggaggacccaggagctcctggtgcctctgccagtgactggctcccatggctgtgagcaaccacaccccctttctgtgcctcagtttcctctcgtgtatgtgaaataaacaagagagactggataagctctgacacgaagccttaggcttgggggcatggaggtgactgtcagctgggattggagaattggggtggagggcagaggctggtagcgaggggccgacttagagccaggagggcagggccaggtgtggtggctcacgcctgtaatcccagcatttgggaggctgaggtgggcggatcacttgaggtcaggagttcaagaccagcctggccaacatggtgaaaccccatctctactaaaaatacaaaaacaaaattagccaggcatggtggtggacacctgtaatcccagctactcaggaggccaaggcaagaaaatcacttgaacccaggagatggaggttgcagtgagtcaagatcgcaccactgcactccagcctgggtgacagagtgagactgtctcaaaaagaaccaggagggcacatgggcatggggagtgatgaaccagagaaagctgctgtctttctgggcaagtgccaagcaacggatcacccttgacccctaggaaagaaaaagaagaaaacccgaggtgatcattttaaacttcgcttccgaaaaaactttcaggccctgttggaggagcaggtgagaggagggtcggcctgggaggaccccacagggaaggggtgagcctggcccgggcaggtgttcgctgcgtgggtgggcggaggagttctagagccggccccttgtctctgcagaacttgagtgtggccgagggccctaactacctgacggcctgtgcgggacccccatcgcggccccagcgccccttctgtgctgtctgtggcttcccatccccctacacctgtgtcagctgcggtgcccggtactgcactgtgcgctgtctggggacccaccaggagaccaggtgagcatgagacctgctgtccactcccactccctccttcccacagcctccccagacctctctcccctcatcctggcttcccctctgtctgcaggtgtctgaagtggactgtgtgagcctgggcattcccagagaggaagggccgctgtgcactgcccggccttcagaaagacagaatttcatcacccaatgcagggggagctcttcctggaccaagggaggagccgctcattcacccaacaaaactgtgtcttatctgccaggaaagaccagcctcactcctgggaactgtctggcaggtaggctgggccccccagtgctgttagaataaaaagcctcgtgccggaagccttcctgtttggtcgtggtgtgtttgaggtgatggtaatgggtcacccgtctctcctgctcacggctctgtctctcttcctcctgcctcccactcacccctgccaccgtccgcccctctgtgtccctgatcgcgagagattctgtcccattttcctgccacccccgagcccctgccctccttggctgcttctttaagtctttttggttattgatttagttgtttaaactattttatttatttattagagacagggtctcgcactgtaacccaggctggagtgcagtggtgcggtctcagctcactgcaacctctgcctcccaggttcaagggattctcctgcctcagcctcccaagtaactgggattacaggtggccaccaccacgcccaactaatttttttatatttttagtagagttggggttttgatgtgttggccaggctggtcttgagctcccaacctcaggtaatccacccgcctcggcctaccaaagtgctgggattataggcgtgggccactgcgcccggccatgtttttcctttttatttctccagttgctatattttcaaagtacagctaggttccagggatgggtgacagggaggtggcgggaggactgtggaacatgtgcagaatggtaccgcggagggaccttggcctgtcttcctctcaaggtacttaggcaaaggggagtggtccttgtccttggtgctgccagactgagggagcagttgtccttaggaagcccctgttaccaggggctgcagtgtgtgaagttattagcagtacggaaaacccagaagtttctgggaaacatgggggaattcatggtatagagcaaatttgtccaacctgcagcctgcatgtggcccaggatgactttgaatgcagcccaacacaaattcataaactttcttaaaacattatgagattcttttgcgatttttttttcttttagctcatcagctatcgttaatgttagtgaattttatatgtctcctaagacaattcttcttccaatgtggcccagagaagccaaaagagtggacacccctggattagaggcccctggtctgggtggccctgggtgctgtgactcatgtgaacttcttatacccataccttgtctaaatgaattgcaagctgtaatctcgtaaggtccttttggttgctgtattaaggcccagaaccctgttaacctcatagcaaagcaagtgcttagtaagtatccattgaacagatggcctcattaagatattagagagatccagcaaagtcaacgttaggcaccattttttttttttctgagatgcagtctcgctctgttgcccaggctggagtgcagtggcgccatctcagctcactgcaacctccacctcccaggttcaagtgaccctcatgcctcagccttctgagtagctgggattacaggcgcctaccactatgcccagctaatttttgtattttttgtagagatggggtttcaccttgttggccaggctggtttcgaacttctgacctcagatgatccacccgcctcggcctccaaagtggtgggattacaggtgagagccaccgtgcctggccagtcaccatgttaggccttcacttttgggtactttcctgggggttggctcaagtattccccagggttttcttgaggaactgcctccctttctctgaagacttgcagctgggcaggcagtactggttttggtgtcagcttcatgttggggggctgatgtcaggattcagagacaatccaggtgaggtggtttttcaagatggctcaaaaatctttgtcctcccattaaaaagtggagtgttacggccaggcaaggtggctcacgcctgtaatcccagcactttgggaggctgaggtgggcagatcacttgaggtcaggagtttgagaccagcctggccaacatggtgaaaccctgtctctactaaaaatacaaaaattagctgggcatggtggcgtgtgcgtgtagtcccagctactctggaggctgaggcagcagaatcgcttgaacctgggaggcggaggttgcagtgagccgagatcacgtcattgcactccagcttgggcaacagaatgagaccccggctaagaaaaaaaaaaaggccgggtgcagtggctcacgcctgtaatcctaacactttgggaggccaaggcaggtggatcacctgaggtcgggagtttgagaccagcctggccaacatggtgaaaccccatctctattgaaagtacaaaaattagccgggcgtggtggtgggcacctgtaatcccagctacctgggaggctgaggcaagagaatcgcttgaacccgggaggtggaggttgcagtgagctgagatctcaccattgcactccagcctgggcaacagagtaagactccatctcaaaaaaaaaaaacaaaaaaaacaaaattagccaggcatggtggcatgtgcctgtaatcccagctactcaggaggctgaggcaggagaatcgcttgaacccgggaggcagaggttgcagtgagctgagatcgcgccattgcattccagcctgggcaacagagtgagactccgtctccaaaaagaaaagaaaagactatttcggggctgaacaacaccatctgtgcttctccgtggcatcctaggctgggattcccgtccagcttgcactgtcagcacaatctctttggattctttcctaacttagctgactgtttcatattttgtggttataaaaagaggcacactggttctgttcaatgtaagatttctctccccagtgacgtactccgccttccaaaatgaccttgagaggacagggctggaccctgaaggcctctggttgctctctgatgccatccatgacattctctaggaagctgagtccagggactttggacccccattgaaaaaccataattcggccaggcaggtggctcacgcctgtaatcccaacactttgggagcccgaagcaggcggatcacttgaggtcaggagttcgagaccagcctggccaacatggtgaaacccagtctatactaaaattacaaaaatcagctaggtgtggtggcacacgcctgtaatcccagctatttgggaggctgaggcaggagaattgctggaacccaggaggcagaggttgcagtgagctgagatcacgccattgcactccagcctgggtgacagagcaagactcagtctcaaaaaaaaaaagaaaaaagaaagaaaaccataattccccaaggacagcctgaaacaattttgccagcttccttcatgctagggagattttttcctaccttgggagacacctggtctttgggaaatgtatactctgctctaaggttctagaaggtaaacctagtgtcatgtgataagggcagcacccccctttcctgggagcacagaggaggaagaggttggtgccagggtttggaagtgtggacacgggtggttactcctaaaaacttcaatgaggctgggtgctgtggctcaggcctgtaatcccaacgctttgggaggctgaggcaggagggtctcttgatcctagtagtttgagaccagcttgggcaacacagggagaccccatctctacaaaaaatttaaaaattggccgggcacggtgactcatgcctgtaatctcagcactttgggaggccgaggcaggcagatcacgaggtcaggagatcgagaccatcctggctaacacggtgaaactccgtctctactaaaaatacaaaaaattagccggcctggtggcgggcacctgtagtcccagctactcgggaggctgaggcaggagaatggcgtgaacccgggaggcggagcttgcagtgagccgagattgcgccactgcactccagcctgtgcgacagatcaagactccgtctcaaaaaaaaaaaaaaaaaaaaaattaaaaattagccaggtatggtggcgcacacctttagtcccagctactcagggaaggatggcttgggcctgggaggttgaggctgtggtgggcagcaatcactttactgcagtccaacctgggcaacagagcaagaccctgtctcaaaacaaaacaaaaaaaagatcttgattttaattgatgtgagttaggagagccaagaattctgggaagcagggcaattgtgtcttcagtcttggagaaatcctggtctgagtactaactagtggtgtagctcggacaggtcacttcacctctctgatgctgttggagccatgctgcagctattgcaagaattcaatacacctagcccgagacctgacttctagtgagtacgcagcaaatgagagcgcttgatgatactgccaagacctaaagcatgttgttgagtttttcacagccagctccctggggactaaacccagcacctggcccaattgtcaggtagccagcagtttgcatttacatttctttttttgagacagagtgtcactctgtcgctcaggctggagtgcagtggcatgatcttggctcactgcaacctccacctcccaggttcaagtgattctcgtgcctcagcctcctgagtagttagaattacaggtgcccaccaccatgccccgctagtttttggatgttttttattttttcttttaatttttttgagacagggtctcactctgtcacccaagctggagtgcagtggcacgatctcagctcactgcaacctctgcctccctggttgttcaagcgattctcctgctgcagcctcctaagtagctgggataacaggcacccgccacctcgcctggctaataattttgtatttttattagagatggagtttcattatgttggccaggctggtctcgaactcctgacctcaggtgatccgccaaccccggcctcccaaagtgctgggattacaggtgtgagcctctgcgcccggccagtggtttacatttgtgtttgctcctaggctgtgggttctttgagggcacaggcccactccagcttctagcagggaagcagctcagtatgtgttctgaaaaagggcaaatgtctccataaccctttgtagtcacatgtccacccgggaagaaatcaaaactcaacctggtgaccatattaaggcacacacttaaatgccgagttgtaaaactgggcacaggttgtttttcctgaagggggaagaagtgagaagaggcaggctggtgttgggaaaaggaggtagacatgtgggggtggcacactgaagacttttggaagtgcctggaggtgtcagcagttgtaattgacttttgtcttccttcactatagggagcactctcatggcctttcaggattgtaagacactttttgtttgtttttcaagacggagtctcgctctgtcgcccaggctggagtgcagtggcacaatctcagctcactgcaatatccatttcccaagttcaagcaattttcctgcctcagcctcctgagtagctgggattataggtgtgtgccaccactcctggctaatttttgtatttttagtagagttggggttttgccatgttggccaggctgctcttgaactcctgacctcaggtgatccgcccaccttagtctcccaaagtgctgggattacaggtgtgagccactgctcctggccctaatttttatttttttagtagagatggggtctcgctacgttgcccaggttggtcttgaattcctgggcttaagggattctcctgccttggcctcccaaagtgctgggattatggccatgatccactgagcctggcctgtaaaagacacttctactgagcctggcctgtaaaagacagaagacggtgtggaaggagaggattgcagttggtcacagacttgttggccacaggaccctttatgtagggagcagctcaaaaggcaccagggcatctggccaggcgcagcggctcacacttgtaattccagcactttgggaggctgaggcaggcggattacctgaggtcaggagtttgagaccagcctggccaacatggtgaaaccctgtctctactaaaaatacaaaaaaattagctgggcatggtggcgcacgcctgtagtcccagctacttgggaggctgaggcaggagaatctcttgaacctgggaggccgaggttgcagtgagccaacatcgcaccactgcactccagcctgggtgacagagcaagactctgtctcaaaaaaaaaaaaaaaaaaaaaaaaaaaagcaccagtgtttctggctggagcccagccaaaaggatc